(19) United States
(12) Patent Application Publication
(10) Pub. No.: US 2012/0232025 A1
(43) Pub. Date: Sep. 13, 2012

(54) SAFENER DRUG COMBINATIONS FOR USE WITH NMDA ANTAGONIST DRUGS

(76) Inventor: John W. Olney, St. Louis, MO (US)

(21) Appl. No.: 13/227,945

(22) Filed: Sep. 8, 2011

Related U.S. Application Data
(60) Provisional application No. 61/381,069, filed on Sep. 8, 2010.

(51) Int. Cl. A61K 31/7048; A61K 31/551; A61K 31/498; A61K 31/4745; A61K 31/428; A61K 31/472; A61K 31/53; A61K 31/55; A61K 31/5415; A61K 31/4168; A61K 31/46; A61K 31/135; A61P 25/00; A61P 25/18; A61K 31/513

(52) U.S. Cl. 514/23; 514/274; 514/220; 514/249; 514/292; 514/367; 514/307; 514/242; 514/217; 514/226.2; 514/401; 514/304; 514/646

(57) ABSTRACT

Prolonged administration of subanesthetic dosages of ketamine, which suppresses activity at NMDA receptors, can provide a damaged central nervous system with an opportunity to use its innate healing processes to “reset” NMDA receptors which were pushed into an unwanted hyper-sensitized state by unusually high activity. However, such treatments can cause permanent brain damage, if the ketamine dosage is too heavy or prolonged. Certain types of “safener” drugs have previously been identified, which can block or at least reduce those unwanted side effects. It is disclosed that if two classes of safener drugs are combined, which will simultaneously suppress activity at both (i) muscarinic acetylcholine receptors, and (ii) the kainate and AMPA classes of glutamate receptors, those safener drug combinations can provide exceptionally potent and reliable safening activity, which can enable the safe use of potent NMDA antagonist drugs for a number of highly beneficial purposes.

### RELATED APPLICATION

This application claims the benefit, under 35 USC 119, of provisional application 61/381,069, filed on Sep. 8, 2010.

### BACKGROUND

This invention is in the field of pharmacology and neurology, and relates to the use of drugs that suppress or increase neuronal activities in mammalian brains.

For purposes of discussion herein, the relevant background art is divided into two major categories. One category, in the subsections directly below, addresses known facts and agreed-upon conclusions, concerning neurons, neurotransmitters, and how neural networks interact and regulate themselves with regard to NMDA receptors and the types of NMDA antagonist drugs that are of interest herein.

The second category is presented under the heading, “Competing Schools of Thought”. This set of art, although published in respected and refereed scientific and medical journals, has not reached a level of consensus, among experts, about agreed-upon facts. Instead, these articles set forth disparate and in some respects conflicting and contradictory theories, hypotheses, and proposals for research and treatments.

The need to have a working knowledge of both sets of prior art, while understanding that they are separate and distinct from each other, is effectively illustrated by the current state of the art in treating serious neurologic disorders. Despite everything physicians and researchers have learned about the brain, there still are no adequate methods for preventing or curing any of the most important disorders of the nervous system, including stroke, epilepsy, Alzheimer's disease, Parkinson's disease, Huntington's disease, amyotrophic lateral sclerosis, schizophrenia, severe depression, neuropathic pain, post-traumatic stress disorders, etc.

Accordingly, since this technical field focuses on extraordinarily difficult and intractable disorders of the nervous system that have defied all efforts to find cures, anyone who wishes to understand the state of the art, in this field of science and medicine, must be able to distinguish between what is actually known, versus what has merely been proposed and suggested as a theory, hypothesis, or hope.

### Factual Foundations: Neurons, Neurotransmitters, NMDA Receptors, and Neuronal Networks

This first portion of the Background section attempts to summarize facts and discoveries that have reached a state of agreement and consensus among medical researchers and clinicians who specialize in neurology and neuropharmacology.

Since the human brain is extraordinarily complex, this summary section is merely a brief introduction and overview which attempts to call attention to certain facts that are relevant to this invention. A huge amount of additional information (with illustrations) is available, at any desired level of detail and completeness. The chapters on the nervous system in any respected textbook on physiology (such as any recent edition of Guyton & Hall, Textbook of Medical Physiology, the most widely-used physiology textbook in medical schools in the US) offer a good mid-level summary. For those who wish to go deeper, the most widely used textbook on neurology, Principles of Neural Science, by Eric Kandel et al, was more than 1400 pages long when published in 2000 (fourth edition); however, a great deal of research in neurology and neuropharmacology has been done since then, and a fifth edition is scheduled to be published sometime in 2010.

In addition to medical textbooks, a large number of published articles focus on NMDA receptors, NMDA antagonist drugs (i.e., drugs that block or suppress activity at NMDA receptors), and potential uses for NMDA antagonist drugs. Review articles describe a field of research rather than presenting original research findings, and they can be easily located by searching the National Library of Medicine database, which is free to users. Early review articles, which helped establish a foundation of knowledge in this field, include, for example, Rothman and Olney 1987; Olney 1989a and 1989b; Choi 1992; Lipton and Rosenberg 1994; and Olney 1994. More recent review articles, many of which focus on potential medical uses for NMDA antagonist drugs, include Sanacora 2003 and 2009, and Hardingham 2009.

### Glutamate Neurotransmitter System;

### NMDA and Non-NMDA (KA and AMPA) Receptors

Glutamate (the ionized form of glutamic acid) is an amino acid that functions as a neurotransmitter in the central nervous system (CNS, which includes the brain, spinal cord, and retinas). CNS neurons communicate with one another through a synapse (or synaptic junction) between a signal-transmitting neuron and a signal-receiving neuron. Glutamate is referred to as an excitatory transmitter, because when a glutamate molecule is released into the synaptic junction, it stimulates (excites) the receiving neuron to undergo a “firing” event (also called a depolarization event, a nerve impulse or signal, or similar terms). Glutamate is the predominant and most important excitatory neurotransmitter in mammalian brains, while acetylcholine (ACh) ranks second. Their excitatory signaling is counterbalanced and modulated by several types of “inhibitory” neurotransmitters, including gamma-amino-butyric acid (GABA), dopamine, norepinephrine, and serotonin.

There are two major families of glutamate receptors, called ionotropic and metabotropic receptors. Of primary importance for the present invention is the ionotropic family, within which there are three receptor subclasses. Each sub-class is named after a certain type of “probe drug” which selectively activates that subclass. NMDA receptors were named after a compound called N-methyl-D-aspartate (NMDA), because NMDA strongly activates that subclass of glutamate receptors, without triggering activity at any other glutamate receptor types. NMDA does not normally exist in a mammalian body, and if administered to an animal at any substantial dosage, it will cause convulsions, since it triggers uncontrolled firing of large numbers of neurons in a mammalian brain.

A second subclass of glutamate receptors is called kainic acid (KA) receptors, because they are activated by kainic acid, another probe drug that does not normally exist in mammals. These receptors are also called kainate receptors, since kainate is the negative ion that is generated when a hydrogen proton (H⁺) dissociates from kainic acid.

A third subclass is called AMPA receptors, because they are activated by a specific type of amino-methyl-propionic acid compound known as AMPA.

The KA and AMPA sub-classes of glutamate receptors are less widespread than NMDA receptors, and a number of probe compounds activate both classes of KA and AMPA receptors, without activating NMDA receptors. Therefore, KA and AMPA receptors are often referred to collectively as “non-NMDA” receptors, or as “non-NMDA glutamate” receptors. KA and AMPA receptors and their components also are designated by several other acronyms, but these can be confusing and are not used herein, for three reasons:

(1) Some of the acronyms have changed as research has progressed, but not all researchers and authors begin using the new acronyms simultaneously. For example, kainate receptors were initially known as GluR5 receptors, and some authors apparently still refer to them by that acronym, but other authors now refer to them as GluK1 receptors. Similarly, GluR6 receptors are now referred to as GluK2 receptors, by some but apparently not all authors.

(2) Some acronyms in this particular field refer to entire receptor complexes, while other acronyms refer to receptor “subunits”. Each receptor subunit comprises a single protein or polypeptide chain, and a limited number of protein molecules (typically about 4 to 6) are assembled into a receptor. This topic is further complicated by the fact that a complete and functional receptor complex includes both (i) an ion channel subassembly, which is made up of multiple protein subunits, and (ii) a neurotransmitter-binding subassembly, made up of additional but different subunits, which controls the opening and closing of the ion channel subassembly.

(3) Finally, some acronyms refer to proteins, while other acronyms refer to gene sequences which encode those proteins.

Accordingly, to avoid additional complexity, acronyms such as GluR5 or GluK1 are not used herein. To understand this invention, it is sufficient to be aware that:

(1) KA and AMPA receptors are the two classes of non-NMDA receptors that are activated by glutamate; and,

(2) a number of drugs have been discovered, and are known to experts in this field, which will suppress activity at KA receptors only, or at AMPA receptors only, or at both KA and AMPA receptors, without also suppressing activity at NMDA receptors. Because this field of drug research is complex, and is important to this invention, it is addressed under a separate heading, “Drugs That Suppress Activity at Non-NMDA Receptors”.

Returning to a general introduction to the glutamate system and glutamate receptors, NMDA receptors are abundantly and widely distributed throughout the brain and spinal cord, in all mammalian species. One of the reasons why NMDA receptors play major roles in a number of crucial neurologic functions (including cognition, learning, and memory, as well as processing of sensory information, including pain) arises from the fact that NMDA receptors have a special property, which is not present in any other types of synaptic receptors, and which allows NMDA receptors to undergo enduring changes in their sensitivity and response time. If an NMDA receptor is stimulated repeatedly and/or at a high frequency by the natural neurotransmitter (i.e., glutamate), it causes the receptor to develop a sustained state of heightened sensitivity. In this state, even a slight stimulation of the receptor will elicit a maximal response. Accordingly, NMDA receptors that have been placed into this altered state are often referred to by terms such as hyper-sensitive, hair-trigger, or “kindled”.

This change in NMDA receptor sensitivity and reactivity is referred to by the scientific term, “long term potentiation” (LTP), because it creates a long-lasting change in how an NMDA receptor behaves and performs. It also is referred to as a “plastic” alteration; this term implies that if something is given a new shape, structure, or trait, it will remain that way even after the manipulating force or factor is removed (rather than returning to its original shape, structure, or trait, which would be elastic behavior).

An analogy from everyday experience may be able to help some readers grasp why NMDA receptors will shift into a hyper-sensitized fast-response mode, if they are in “heavily traveled” neuronal pathways and circuits. If a house has both a front door, and a sidewalk gate with a buzzer, and if people begin coming to the sidewalk gate with unusual frequency and ringing the buzzer, the residents of the house likely will grow tired of having to go all the way out to the front gate, each and every time a visitor appears at the gate. So, the people who live in the house will simply leave the sidewalk gate open, and will allow visitors to come to the front door, and ring that doorbell, if they want the enter the house. This is comparable to what neurons do, with NMDA receptors that must be opened with unusually high frequency. Indeed, some neurologists refer to NMDA receptors as having “outer vestibule” components, which initially involve channels that are relatively narrow and tightly constrained, unless and until an NMDA receptor undergoes a transformation (apparently involving a specific protein component called the NR3A protein), which effectively opens up and converts the narrow entryway into a much more open channel that will allow rapid flow through the channel (e.g., Wada et al 2006).

These types of lasting changes, in NMDA receptors, play important and useful roles in learning, memory, and other neurological functions. However, if they arise in neural circuits and pathways where they should not occur, hyper-sensitized NMDA receptors can create or aggravate extremely severe pathological conditions, such as neuropathic pain, as described below.

It should be noted that different types of neurological problems can arise, depending on whether the neuronal pathways and circuits that involve NMDA receptors have become either too active, or insufficiently active. Accordingly, the abbreviations “NR/hyper” and “NR/hypo” are used as follows:

(1) If a pathological condition is caused or aggravated by excessive levels of activity at NMDA receptors, the condition is classified and labeled as an NR/hyper condition. These types of disorders, which are described in more detail below, can be treated in a direct manner by NMDA antagonist drugs, since (by definition) NMDA antagonist drugs are drugs which will reduce activity at NMDA receptors.

(2) If a pathological condition is caused or aggravated by low or inadequate levels of activity at NMDA receptors, the condition is classified and labeled as an NR/hypo condition. The most important type of mental disorder which is assumed and believed to involve chronic NR/hypo activity is schizophrenia. This belief arises largely from two known facts: (i) ingestion or administration of an NMDA antagonist drug, such as phencyclidine or ketamine, will create a drug-induced NR/hypo condition, which will then create symptoms and effects that resemble the symptoms and effects of schizophrenia; and, (ii) limited testing of phencyclidine or ketamine on schizophrenic patients (which is now prohibited, as described below) tended to aggravate and intensify their symptoms and/or trigger psychotic episodes. Accordingly, schizophrenia, and other neurological disorders that involve NR/hypo conditions, are not suited for treatment by NMDA antagonist drugs, since NMDA antagonists will aggravate and worsen an NR/hypo condition, rather than alleviating it.

Accordingly, the discussion immediately below summarizes three major types of neurologic disorders which involve NR/hyper activity, and which are suited for treatments using NMDA antagonist drugs (provided that effective dosages of “safener drugs” are also coadministered to such patients, to reduce or prevent the neurotoxic damage and psychotomimetic stresses that are imposed on brains when large or sustained dosages of potent or moderately potent NMDA antagonist drugs are used). These three classes of neurologic disorders are not an exhaustive list of all known disorders which arise from NR/hyper activity; however, they provide a good introduction to the types of neurologic problems and disorders that can arise, when activity at NMDA receptors becomes so excessive that it generates serious medical problems.

For convenience, terms such as dysfunction (or dysfunctional), disease, and disorder, are used interchangeably herein. All of these terms refer to a mammalian nervous system that is suffering from a problem or disorder involving an unwanted NR/hyper status at a level which can benefit from a sustained medical intervention using an NMDA antagonist drug, such as ketamine.

It should also be noted that any such medical treatments should and must be carried out under the close supervision and monitoring of a physician, in a hospital or clinic that allows constant and continuous monitoring of various vital functions (blood pressure, heartbeat rate, etc.) as well as the mental state of the patient (where symptoms or displays such as agitation, disorientation, irrational behavior, and hallucinations are of particular concern). The neuroactive drugs of interest herein are prescription drugs that can be prescribed and lawfully administered only by physicians, and the types of treatments described herein will use dosage-duration combinations that are intended, quite literally, to permanently alter a patient's nervous system.

Excitotoxic Brain Damage, after Loss of Blood or Oxygen Supply

One of the most important classes of medical treatments that can provide enormous medical benefits, if an NMDA antagonist is coadministered along with an effective safener drug combination as described herein, involves treatments which can prevent or at least minimize the types of rapid and acute brain damage that are caused by a disruption of blood or oxygen supply to the brain. Such disruptions, often referred to as ischemia (which refers to inadequate blood supply) and/or hypoxia (inadequate oxygen supply), can be caused by various types of acute crises, such as stroke, cardiac arrest, head injury or trauma, blood loss, near-drowning or suffocation, carbon monoxide poisoning, etc. The extent and severity of the brain damage caused by these types of crises can be aggravated and worsened by a process that was first described and characterized by one of the inventors herein (Olney 1969, 1974), who coined and created the term “excitotoxicity” to describe this particular type of brain damage.

As a brief overview, the type of brain damage that is classified as “excitotoxic” will arise if the supply of blood or oxygen to the brain is disrupted, such as during a stroke, cardiac arrest, head injury, suffocation, or similar crisis. This occurs due to the following factors:

(1) the disruption of blood and/or oxygen supply leads to a loss of energy, which is needed to drive and enable various cellular functions inside the brain;

(2) one type of function that is vital, inside a brain, involves a “glutamate transport system”. Inside a brain, glutamate is released, as an excitatory neurotransmitter, into the fluid that fills synaptic junctions between signal-transmitting neurons, and signal-receiving neurons. After a molecule of glutamate contacts and activates a receptor protein on the surface of a signal-receiving neuron, the glutamate is released by the receptor, and it becomes, for a brief period of time, free (or uncleared) extra-cellular glutamate, floating in the watery fluid that fills the synapse. To prevent that free glutamate from re-activating receptors in ways that would trigger unwanted nerve impulses, the glutamate transport system effectively “grabs” any extra-cellular glutamate molecules, and pumps them back inside neurons (or glial cells), so that the glutamate can be reused again, in subsequent nerve signal transmissions;

(3) the glutamate transport system requires energy, to drive the pumping of free glutamate back into cell interiors. If the energy supply in the brain (or some portion of the brain) is cut off, due to a loss of blood or oxygen supply, the glutamate-pumping mechanism will fail, and free extra-cellular glutamate will begin to accumulate in uncontrollable quantities, in the fluid that fills the synaptic junctions between neurons;

(4) if “uncleared” glutamate begins to accumulate in the synaptic junctions between neurons, it will begin to repeatedly and uncontrollably contact and activate glutamate receptors. These receptor activations will cause the affected neurons to begin “firing” in an excessive and uncontrolled manner;

(5) because of certain factors involved in their cellular physiology, neurons cannot withstand uncontrolled high-frequency impulse signals, for a prolonged period of time. Instead of driving neurons into a dormant or inactive state, where they simply will not respond for a while, exhaustion and depletion will literally begin to kill the affected neurons. This process is called “excitotoxicity”, because the neurons are being excited (by uncontrolled activation of the glutamate receptors in their synapses) until they die, due to toxic levels of excitation and exhaustion.

Furthermore, because any hyper-activated neurons are being driven to release abnormally large quantities of their own neurotransmitters at the “downstream” synapses they share with other neurons, additional portions of the brain (which, in many cases, were initially unaffected by a stroke or other injury) get pulled into the toxic whirlpool or cascade, and begin to suffer from a type of damage that is often called “penumbral” damage.

The type of excitotoxic brain damage that occurs during and after an acute crisis (such as a stroke, cardiac arrest, etc.) is discussed in more detail in review articles such as Rothman and Olney 1987, and Choi 1992. Because it involves NR/hyper activity, it offers one example of a medical condition that can be treated by NMDA antagonist drugs, provided that additional steps are taken, as described herein, to avoid the unwanted neurotoxic and psychotomimetic side effects of the NMDA antagonist.

It should also be noted that glutamate-driven excitotoxic damage to the brain can be created or aggravated by certain other conditions. For example, in a prolonged epileptic seizure (status epilepticus), uncontrolled over-excitation of certain brain regions (effectively causing a continuous convulsion) depletes the energy supplies of the brain faster than they can be replenished, which jeopardizes the energy supply that is needed to run the pumping system that clears and removes free extracellular glutamate. In a patient suffering from hypoglycemia, there are inadequate supplies of sugar (especially glucose, the only sugar molecule that most brain cells are capable of using as a fuel) in the blood; again, this condition jeopardizes the energy supply that is needed to run the glutamate pumping system. In asphyxia (which can occur during a near-drowning, near-suffocation, etc.) or in carbon monoxide poisoning (which cripples the ability of blood hemoglobin to carry oxygen), the loss of oxygen supply in the blood can also cut off the energy supply that is needed to run the glutamate pumping system. Accordingly, all of those conditions can lead directly to excitotoxic brain damage, driven or aggravated by uncleared extra-cellular glutamate inside the brain or spinal cord.

Accordingly, the type of excitotoxic brain damage that occurs during various types of acute crises is one type of problem, involving NR/hyper activity, which can be treated by using NMDA antagonist drugs, if adequate means can be provided for preventing the unwanted side effects of the NMDA antagonist drugs.

The next two sections describe two other types of neurologic problems that also involve NR/hyper activity, which also can be treated by NMDA antagonist drugs, using different modes of administration. Rather than involving acute crises, these two problems involve aberrant and unwanted “plastic” changes in NMDA receptors, which can lead to severe problems which will last indefinitely.

### Neuropathic Pain

As mentioned above, if an NMDA receptor is activated with high frequency, it can undergo a “long-term potentiation” (LTP) change, which will place it in a “hyper-sensitive” status (which can also be referred to by terms such as hair-trigger, kindled, etc.). While such changes are very useful for processes such as learning and memory, they can cause terrible problems, if the hyper-sensitive NMDA receptors are located in neural pathways that carry pain signals. For example, if a person is injured or infected in a manner that generates severe pain for a prolonged period of time, a condition of chronic pain may arise, which will remain long after the initial injury or infection has healed. Pain is classified as “neuropathic” if it remains after an initial injury, infection, or similar problem has healed, and if a level of serious pain becomes a lingering problem that appears to involve an unwanted and pathological alteration in one or more parts of a patient's nervous system.

Neuropathic pain conditions are among the most difficult and intractable of all medical problems. Conventional pain remedies (including morphine and other opiates) that are effective in relieving most other types of pain are usually ineffective or, at best, only minimally effective in relieving neuropathic pain. Those few medicines which can provide substantial relief from neuropathic pain, at least for a while, tend to be severely addictive, notably including oxycodone, which is sold in an extended release formulation under the trademark OXYCONTIN.

Because NMDA receptors are the only class of neuronal receptors that are known to undergo a “plastic” (or long-term potentiation) change as described above, which can lead to “kindled” neuronal pathways that may generate, send, and carry unwanted pain signals in response to even very mild stimuli, they have long been suspected and assumed to be involved, to at least some degree, in essentially all cases of neuropathic pain. Therefore, it was entirely logical to test NMDA antagonist drugs to evaluate whether and to what extent they could reduce neuropathic pain, and numerous researchers began doing so in the early and mid 1990s. Most of the research in this field has been conducted with ketamine, because it is approved for use in human therapy, and is well known to have pain-suppressing properties when used in anesthesia.

The early results from ketamine studies on neuropathic pain in humans were very inconsistent. One of the major problems that was encountered, from the very outset of such tests, was that whenever a dosing regimen was used that provided actual and substantial relief from neuropathic pain, a high incidence of serious side effects (primarily hallucinations, and psychotic episodes or behaviors among some patients) were typically encountered, and often led patients to withdraw from those studies before data-gathering could be completed.

For example, Eide et al 1994 compared a single intravenous injection of ketamine (0.15 mg/kg) versus a single IV injection of morphine (0.075 mg/kg). Subsequently, in a followup test reported in Eide et al 1995, several patients were selected, based on their positive responses to single injections of ketamine. Those selected patients were given ketamine by subcutaneous injections, over a span of 7 days and nights. Although reduced pain was reported, Eide et al concluded that the 7-day ketamine treatments created “intolerable side effects”.

In Backonja et al 1994, six patients were tested with single-dosage injections of ketamine. They reported relatively brief reductions in pain levels, with side effects that were “mild and well tolerated”. One patient was then tested using continuous subcutaneous infusion. It provided “no additional improvement in pain control but caused intolerable cognitive and memory side effects”. Accordingly, the continuous infusion test was terminated after only one patient was tested.

In other tests (Fitzgibbon et al., 2002; Rabben et al., 2001; Mitchell 2001), various approaches for limiting the dosages and durations were explored, in an effort to minimize side effects. However, despite their best efforts, none of those researchers were able to devise any treatment protocols that were able to provide sustained relief from neuropathic pain, without also causing unacceptable side effects.

Those results caused the earliest researchers to reach pessimistic conclusions that NMDA antagonist therapy was not a tenable approach for treating neuropathic pain, in healthy patients. However, others persisted, and a number of researchers focused on patients who were dying from terminal illnesses, such as advanced and inoperable cancer. Examples of such treatments using ketamine are described in articles such as Klepstad et al 2001 and Kannan et al 2002. Clearly, during the final days of a terminal disease, in a patient who will die soon regardless of what is done and who otherwise would be in agony, concerns about possible neurotoxic side effects are much less important than pain control. A review article published not long afterward (Bell et al 2003) concluded that the data and evidence were not sufficient to make any reliable recommendations for dosing protocols, when ketamine was used to treat cancer pain.

Pain which is being caused by terminal or other cancer is not classified as “neuropathic” pain, at all. Therefore, this digression, to briefly mention ketamine treatments for pain in cancer patients, does not relate directly to neuropathic pain treatments. However, it is worth noting that, even in terminally-ill patients who are rapidly approaching death, where concerns over long-term brain damage become totally irrelevant, pain specialists still have not been able to develop any consistent and acceptable treatments or dosage protocols, using ketamine.

Other small-scale tests of ketamine for relief of actual neuropathic pain used dosage regimens that were limited in various ways, such as by making the dosages intermittent rather than continuous. As examples, in trials described in Fitzgibbon et al 2002, a single infusion of ketamine over 24 hours was followed by once-per-night pills; Rabben et al 2001 reported single intramuscular injections, followed by once-per-night pills; and, Mitchell 2001 reported a series of 21 infusions over a period of four months, which works out to an average of one relatively brief infusion every 6 days.

However, nothing of particular interest has emerged (or is likely to emerge) from any of those studies. A relatively recent review (Okon 2007) concluded “there is no agreement on a single, uniform best ketamine protocol or dose. Instead, various local, idiosyncratic approaches are used.” It is apparent from the Okon review that, because of the extreme difficulty in controlling neuropsychotoxic side effects, most pain specialists avoid using ketamine, except for terminal cancer patients; and, even for that very limited purpose, there is no agreement regarding how ketamine should be administered to terminal cancer patients.

It should also be noted that in various studies reviewed by Okon 2007, antipsychotic drugs (most commonly haloperidol) were tested, in an effort to counteract the psychotomimetic side effects of ketamine. However, those drugs, when given at the same time as ketamine, did not succeed in accomplishing that goal. This is consistent with evidence gathered by one of the applicants herein (Olney), using test animals, which showed that haloperidol, when given at the same time as the NMDA antagonist and at clinically relevant dosages, could not prevent NMDA antagonist drugs from causing the formation of “vacuoles” (discussed below) in the brains of test animals.

Thus, at the present time, there is no safe and reliable way that is used or agreed upon, by neurologists or pain specialists, for using ketamine or any other NMDA antagonist to treat neuropathic pain.

Based largely on work (much of it apparently unreported) done in Germany and Russia over the past 2 decades, a type of treatment referred to as a “ketamine coma” treatment has been tested on a limited number of patients who were suffering from an extremely severe type of neuropathic pain. Those types of extremely severe cases merit some background information to explain why some researchers and physicians have considered it justified to use drug-induced comas, to relieve at least some of the pain being suffered by certain types of patients.

Especially severe cases of neuropathic pain are often referred to by either of two phrases. The earlier term, still commonly used among support groups and internet sites even though it has been superseded and replaced within the medical profession, is “reflex sympathetic dystrophy” (RSD). The term “reflex” implies an involuntary response to some causative event, such as an injury or infection. The term “sympathetic” was included when “RSD” became the standard descriptive phrase, decades ago, because RSD was believed at the time to involve the so-called “sympathetic” nervous system, which is not under a person's conscious control. The term “dystrophy” was included, because the pain is so severe and intense, in people suffering from RSD, that it will disrupt a neuromuscular system so badly that a limb may begin to atrophy and shrivel, or become so severely impaired that it becomes effectively useless. In medical terms, the root word “troph” refers to nourishment, and “atrophy” indicates active shriveling, involving a loss of muscle or other mass. “Dystrophy” is a broader term; it indicates a serious malfunction or disruption, and it can include atrophy, but it can also stop short of atrophy and outright loss of muscle mass.

Recently, RSD has been renamed, by pain experts, as “complex regional pain syndrome” (CRPS). As implied by the words “complex” and “regional”, this diagnosis is not used for simple or localized chronic pain; instead, it is used only in very serious cases, where a severe pain condition has spread beyond a single site and has begun to affect an entire limb, quadrant, or other large and/or growing portion of the body. CRPS cases are further labeled as Type 1 (if the neuropathic pain did not arise from a specific known injury or infection) or Type 2 (if the condition arose from a major injury or infection).

Accordingly, “ketamine coma” therapy was developed in an effort to deal with cases of neuropathic pain so severe that they are ruining a patient's life. The treatment consists of administering ketamine by intravenous infusion, continuously, at a dosage that will render a patient completely unconscious for a period which typically lasts 5 to 7 days, while the patient is kept alive by means of a respirator, feeding tubes, etc.

Because of the high risks associated with ketamine coma therapy, it is prohibited in the United States, and patients who wished to undergo this therapy must travel to other countries to receive it. For a number of years, most such treatments involving US patients were performed in Germany, through a network of European physicians who collaborated with Dr. Robert Schwartzman, in Philadelphia. Their work is described in articles such as Kiefer et al 2007, which reviewed the results of 20 such treatments. However, that review does not address two casualties (one death, and one full-body paralysis) that occurred after 2007, apparently involving antibiotic-resistant bacterial infections that commenced while the patient's body was unable to mount a full-fledged immune response.

Recently, a number of ketamine coma treatments have been performed in Mexico, under the control of physicians who collaborate with Dr. Anthony Kirkpatrick, who leads the RSD/CRPS Treatment Center and Research Institute, created in 2008 in Tampa, Fla. (http://rsdhealthcare.org).

Because of the extensive known risks that are involved, anyone who works with ketamine coma treatments will readily state that it is a last resort, which should be considered only in the most severe cases of unrelenting, excruciating pain. Additional information on these treatments can be found by an Internet search of “ketamine coma”, and via a number of sources compiled at http://fightingagainstrsd.tripod.com/treatments.html.

A third type of treatment was initially developed by Graeme Correll in Australia; it later was expanded and refined by Dr. Ron Harbut in the US, in collaboration with one of the Applicants herein (Olney). It is referred to herein as a “continuous subanesthetic treatment”. It lasts continuously, 24 hours/day, for a span of time such as 5 days, but the “subanesthetic” dosage does not render the patient unconscious. Instead, the dosage is “titered” for each specific patient, by commencing with a low dosage (via intravenous infusion), which is escalated in a stepwise manner until the patient displays neuromuscular impairment (indicated by slurred speech or similar effects), or when the patient reports feeling somewhat inebriated. The dosage is then reduced slightly, to provide a margin of safety, and it is continued for multiple days. During that time, patients generally feel a high level of relief from the pain, which is very pleasant for them. They tend to feel drowsy, and usually have a normal sleep cycle, supplemented by naps. However, they are not rendered unconscious by the subanesthetic dosage they are receiving.

The Correll et al 2004 article was explicitly labeled as a “retrospective analysis”; it summarized treatments that had been given by Correll to 33 patients in Australia during 1996-2002. Dr. Ron Harbut witnessed some of Correll's treatments in Australia, as part of a “Physicians Without Borders” work program, and after Harbut returned to the United States and began submitting grant applications to do additional research on that treatment method, he was instructed by the FDA to contact Olney (one of the Applicants herein), to address questions and concerns about the types of “Olney lesions” which had been detected in the brains of test animals which had been given large dosages of potent NMDA antagonists.

By the time the Correll et al 2004 article was approaching publication, it had been shown from additional animal tests that vacuole formation and similar stress-related symptoms, which initially were reversible if a mild or moderate dosage of MK-801 or phencyclidine was administered, became irreversible and led to permanent neuronal damage, if the NMDA antagonist dosage was increased or repeated. Therefore, a “black box warning” (entitled “Appendix 1: Precaution and Warning”) was appended to the end of that article, describing the recent animal tests, and proposing that a suitable neuroprotective agent, such as an alpha-2 adrenergic agonist such as clonidine, guanabenz, or dexmedetomidine, should be included along with ketamine, if a sustained ketamine infusion therapy was used for treating a neuropathic pain condition.

Subanesthetic ketamine infusion tests have been performed on a number of patients by Harbut, since the appearance of the 2004 article by Correll et al. To a limited extent, some of those treatments are described in published US patent application 2005/0148673, and in Harbut et al 2002.

Although the results of small-scale clinical trials that have been described in print to date do not address or resolve this issue, it is the firm conviction of the Applicants herein that, because this particular type of treatment must continue for multiple days without interruption in order to “reset” the nervous system in a way that can provide lasting relief from severe cases of neuropathic pain, this treatment cannot be done in an optimally and reliably safe manner, with sufficient efficacy for truly severe cases, without using not just one safener drug, but a combination of safener drugs as disclosed herein, to ensure the best possible margin of safety.

Chronic and/or Severe Depression and Similar Conditions

Another condition for which there is a growing body of evidence implicating LTP-altered NMDA receptors, as causative or aggravating factors, involves major depressive disorder. More information on this subject is available in a number of review articles, including Paul et al 2003, Witkin et al 2007, Pittenger et al 2007, and Skolnick et al 2009.

It would seem to be counter-intuitive to suspect or believe that depression can be treated effectively, by administering a drug which further suppresses and depresses the rate of excitatory activity in the brain. Nevertheless, several recent studies have provided evidence which supports the belief that chronic excessive activity, at NMDA receptors, appears to play a key role in causing or aggravating at least some cases of depressive disorders that are resistant to conventional anti-depressant therapies. Accordingly, a number of researchers and clinicians in this field are beginning to develop and propose various theories and hypotheses which might help explain how NR/hyper activity might cause or aggravate depression. The Applicants herein offer one such theory and hypothesis, which is set forth below, under the heading, “Competing Schools of Thought”.

Without requiring any final conclusions on whether the Applicants' theory is correct, the art published to date, concerning the testing of ketamine for treating severe depression, includes the following articles. All of these articles are limited to small-scale trials.

Berman et al 2000 reported that intravenous infusion of ketamine, for 40 minutes, to a small group of depressed patients, provided transient but significant relief from depressive symptoms. Sanacora et al 2004 reported that there is an abnormal increase in the concentration of glutamate in certain regions of the brain in major depression patients. This is believed to be consistent with the NR/hyper hypothesis, since chronically increased concentrations of glutamate are likely to lead to persistent overstimulation of NMDA receptors, which likely would end up causing at least some of those NMDA receptors to shift into a hyper-sensitized LTP-altered status. Also consistent with that hypothesis are the findings of Zarate et al 2004, who reported that treatment with riluzole, a drug that decreases the release of glutamate, is beneficial in major depression. Decreased release of glutamate would have an effect similar to blocking NMDA receptors with ketamine. In each case there would be decreased stimulation of NMDA receptors, which would tend to correct or normalize an NR/hyper state. In a brief report pertaining to only 2 patients suffering from severe depression, Correll et al 2005 reported that prolonged intravenous infusion of a subanesthetic dose of ketamine lasting for up to 4 days provided rapid and lasting relief from depressive symptoms.

These several studies, by different authors and conducted according to varied and in some respects conflicting research designs, have one thing in common: they all support the hypothesis that NMDA receptors are likely to be hyperactive, rather than suppressed, in at least some cases of depression.

However, a study conducted at the National Institute of Health (NIH), published in Zarate et al 2006a, produced evidence that appeared to contradict the “NR hyperactivity in depression” hypothesis. This study was conducted according to a placebo-controlled double blind design to test the anti-depressant efficacy of memantine, a relatively mild NMDA antagonist that also has activity at ACh receptors, as described below. The researchers administered memantine to patients suffering from severe chronic depression, at a dose (20 mg/day) which is the maximum recommended dosage, for treatment of patients with Alzheimer's disease. After 8 weeks of treatment with memantine, the patients showed no signs of improvement in their depressive symptoms.

Zarate et al interpreted their finding as a non-confirmation of the hypothesis that NMDA receptor hyperactivity plays a role in major depression. However, the Applicants herein believe, instead, that the dosage of memantine used by Zarate et al (i.e., the highest dosage approved by the FDA for use in patients with moderately advanced Alzheimer's disease) was actually so low, and memantine is so weak in its NMDA antagonist effects, that any effects that memantine may have had on NMDA receptors, at that dosage, were so minor that they became insignificant.

Because of the apparent contradiction between the memantine results published in Zarate et al 2006, and the results of earlier studies which showed that ketamine had at least some effects on severe chronic depression, Zarate et al conducted a second study on patients with major depressive disorders, using ketamine rather than memantine. That second study involved a randomized, placebo-controlled, double-blind crossover study, in which an intravenous infusion of ketamine lasting 40 minutes was administered to patients suffering from severe and “treatment resistant” depression. This study, which was also conducted at NIH, was designed to provide a rigorous test to either rule in or rule out a role for NMDA receptors in major depression. The results, published in Zarate et al 2006b, indicated that a single intravenous infusion of ketamine provided rapid relief from depressive symptoms within less than 2 hours after the infusion, and in a significant number of patients, remission of symptoms lasted for up to a week.

The Zarate et al 2006b report was lauded by mental health authorities (including the Director of the National Institute of Mental Health, and the Director of the National Institutes of Health) as a major advance in the study of methods for treating depression. For example, the NIH Director, Thomas R. Insel, stated, “This is the first report of any medication or other treatment that results in such a pronounced, rapid, prolonged relief of major depressive symptoms with a single dose. These were very treatment-resistant patients.” The NIMH Director, Elias A. Zerhouni, pointed out, “The public health implications of being able to treat major depression this quickly would be enormous. These new findings demonstrate the importance of developing new classes of antidepressants that are not simply variations of existing medications.” These quotes are available from a news article posted at www.narsad.org, a website run by the National Alliance for Research in Schizophrenia and Affective Disorder (NARSAD), a nonprofit organization that supported Zarate's research. These findings were also written up as an editorial comment in the Sep. 27, 2006 issue of the Journal of the American Medical Association (JAMA). The main significance of the Zarate et al. study, according to the authors of the study and mental health authorities who have commented upon it, is that it proves a principle (i.e., that NMDA receptors form an important target for development of pharmacotherapies for depression). However, both the JAMA and the NARSAD articles explicitly cautioned that currently available NMDA antagonist drugs, including ketamine, are not suitable candidates for antidepressant therapy, because of the serious side effects they are known to produce.

In summary, excitotoxic brain damage, neuropathic pain, and severe chronic depression comprise three groups of major medical problems that can be greatly alleviated, in at least some cases, if NMDA antagonist drugs can be administered safely over prolonged periods of time. However, those three classes of problems are not the only neurologic problems and disorders that are known or suspected to involve NR/hyper activity. Because NMDA receptors are the only types of neuronal receptors that undergo lasting changes in their levels of sensitivity and responsiveness, it is possible (and it is considered highly probable by the Applicants herein) that a variety of other neurologic and/or psychiatric disorders involve, to at least some degree, as a contributing or aggravating factor (and in some cases as a causative factor), NMDA receptors that have been pushed and driven into a hyper-responsive condition involving “long-term potentiation” as mentioned above.

In particular, the types of neurologic disorders which are believed by the Applicants herein to involve NR/hyper activity and/or LTP-altered NMDA receptors, as contributing or aggravating elements or factors, include:

(1) addictions to drugs, tobacco, or alcohol;

(2) obsessive-compulsive disorders;

(3) bipolar and/or affective disorders;

(4) at least some types of phobias;

(5) post-traumatic stress disorders;

(6) at least some cases of eating disorders, including anorexia and bulemia;

(7) at least some cases of epilepsy; and,

(8) various other types of cases involving destructive, pathological, or aberrant disorders or behaviors which generate, arise from, or otherwise involve emotional, sexual, compulsive, or other symptoms (which may include cravings, dependencies, and other displays and manifestations) which appear to be beyond rational control, or which pose challenges and problems so severe that a major drug intervention, which can effectively “rewire” part of the brain, becomes advisable and warranted (preferably in conjunction with counseling or behavioral modification treatments involving a psychiatrist or psychologist who has prior experience working with patients who have undergone these types of drug treatments).

Furthermore, there are various types of disorders in which a pathological condition that begins outside the nervous system (such as diabetes, as one example) is likely to force the central nervous system to respond, by creating and undergoing various responses and adaptations. In some such cases, it may be that the brain would be able to adapt and respond to those disorders in a more stable and effective manner, with fewer discontinuities and maladaptations, if the brain is given a sustained period of relative calm and quiet, during which it can establish a more stable form of homeostasis and equilibrium. The types of highly effective safener combinations disclosed herein can help researchers provide test patients with those types of treatments, to enable them to determine whether a sustained NMDA antagonist therapy can help accomplish those types of medical goals, in any specific categories of such patients.

In addition, it is also believed and asserted by the Applicants herein that at least some patients who suffer from frequent and/or chronic headaches, including migraine and/or cluster headaches, are likely to benefit, to at least some extent, from the treatments disclosed herein.

Accordingly, by enabling prolonged and effective yet safe treatments using ketamine (or other NMDA antagonist drugs with comparable potency levels), the disclosures herein will provide clinicians and therapists with powerful pharmacologic tools that can be used to treat such disorders, and to study such disorders in ways that can measure and evaluate the extent to which LTP-altered NMDA receptors are involved in a variety and range of mental and neurologic disorders. The disclosures herein (i.e., of highly effective safener drug combinations which can eliminate essentially all risks of unwanted neurotoxic brain damage or psychotomimetic stresses due to sustained administration of a potent NMDA antagonist) will enable treatment and diagnostic regimens to be developed and evaluated, in a safe manner, for any neurological disorders which are known or suspected to involve NR/hyper activity as either a causative or aggravating component.

In particular, it should be noted that several types of mental, psychological, or neurologic disorders either overlap with depression, or involve important causative or contributing elements that also play major roles in severe chronic depression. Two specific examples include:

(a) bipolar disorder, which formerly was referred to as “manic-depressive” disorder or behavior, in which a person shifts between periods or episodes of “manic” behavior, and periods or episodes of severely depressed behavior; and,

(b) post-traumatic stress disorder, which usually involves episodes of intense feelings of stress, anxiety, agitation, or panic, alternating with episodes of intense feelings of despair, helplessness, and depression.

Accordingly, bipolar disorder and post-traumatic stress disorder are referred to herein as disorders that are similar to major depressive disorders, for the purposes of this invention. Because of their overlapping nature and the elements they share, comments herein concerning treatments for depression, using a combination of an NMDA antagonist drug and at least two safener drugs, also apply to similar treatments using an NMDA antagonist drug and at least two safener drugs, for bipolar disorder, or for post-traumatic stress disorder.

Furthermore, a number of researchers and clinicians have proposed that slowly-evolving and/or low-grade excitotoxic processes may be causing, aggravating, or accelerating the types of neuronal degeneration that occur slowly, in a number of diseases that are generally referred to as “degenerative” or “neurodegenerative” diseases. These diseases, which usually act over spans of time measured in months or years, include Parkinson's disease, Alzheimer's disease, amyotrophic lateral sclerosis, Huntington's chorea, various forms of senile and other dementia, etc.

However, evidence which would either prove or deny the theory that excitotoxic damage and/or NR/hyper activity may be causing, aggravating, or accelerating any one or more of the above-named degenerative diseases has not reached a level of consensus and agreement, among experts. Accordingly, these disorders are discussed in more detail below, under the heading, “Competing Schools of Thought”. The point that should be recognized, in regard to slowly-progressing neurodegenerative diseases, is that the pharmacologic tools and methods disclosed herein will provide researchers and clinicians with the tools they need, to carry out targeted and focused yet safe research into the roles and involvement of NMDA receptors and NR/hyper activity in any type of slowly-progressing neurodegenerative disease.

Finally, several reports (e.g., Rzeski et al 2001 and 2002; Stepulak et al 2005) indicate that if neuronal activity is suppressed by an NMDA antagonist, certain types of cancers (including brain cancers such as astrogliomas, as well as cancers outside the brain, including lung cancer) can be suppressed to some extent, and will grow more slowly. This cancer-suppressing effect apparently arises from certain types of extra-cellular kinase pathways that involve cellular signaling. The ability of scientists to study the potential use of ketamine therapy, to slow down the growth of malignant tumors (and possibly the spread or activity levels of non-tumorous cancers as well), has been seriously hindered by the psychoactive side effects of ketamine. Accordingly, the safener combinations disclosed herein can both: (i) provide researchers with the ability to conduct more extensive research into the ability of NMDA antagonist therapy to help suppress and treat cancer; and, (ii) enable such therapies to be carried out in a safer and more effective manner, if additional research proves that such treatments are indeed useful.

This completes an overview of the types of problems that can be treated, using prolonged administration of NMDA antagonist drugs, if such treatments can be rendered truly safe without losing efficacy.

### Homeostasis, Set-Points, and Innate Repair Mechanisms

A brief explanation of certain well-known natural processes that play essential roles in animal physiology can help explain how and why the Applicants herein believe and assert that certain types of treatment protocols, involving sustained and prolonged administration of an NMDA antagonist drug such as ketamine combined with two or more safener drugs, are likely to generate better, more effective, and longer-lasting desirable therapeutic effects, in at least some patients (including patients suffering from especially severe forms of various neurologic disorders) compared to a series of discontinuous intermittent administrations of pills or injections. While these types of continuous sustained treatments do not preclude the development or use of various types of intermittent treatment regimens, especially for milder cases of such disorders, the Applicants here believe that continuous and sustained treatments very likely will offer and provide the most potent and effective treatment regimens, for the most severe cases of the types of neurologic disorders disclosed herein. It also should be noted that such cases often can provide the best “proof of concept” demonstrations for a new and innovative therapy, for two reasons. First, it often is easier and faster to obtain FDA approval to test new and innovative treatments on the most severe cases that can be identified, since those are the cases that have not been (and apparently cannot be) adequately treated by any other known therapy. Second, if truly dramatic and never-before-seen improvements can indeed be achieved, even in the most severe and otherwise intractable cases, it tends to quickly remove any lingering doubts about whether such treatments are indeed effective.

The natural processes that are involved center around a process called homeostasis, and the concept of setpoints. Homeostasis refers to the cellular, muscular, hormonal, and other biochemical processes that enable animals to sustain an ongoing and adaptive equilibrium-seeking biochemical stability, in response to constantly-changing food status, environmental conditions, etc. In any healthy animal, if conditions change (such as after a person or animal goes through a period of physical exertion, eats a meal, becomes too hot or too cold, or suffers an injury or infection), the body will respond in ways that enable it to remain within (or to return toward) a range of healthy and stable operating and functioning parameters (which involve factors such as body temperature, heartbeat rate, digestive activity, blood gas levels, etc.).

The term setpoint refers to a healthy, properly functioning, and desirable status or condition. It can refer to the entire body of an animal or person, or it can refer to any particular bone, organ, or other component. The “setpoint” concept is broad enough to apply to nearly any factor that will enable any particular bone, organ, or tissue to function properly; it can include size, shape, structure, morphology, temperature, fluid flow rates, etc., and in most cases, a “setpoint” will cover a range of reasonable and desirable operating conditions, rather than a single exact number. In any vertebrate animal, every bone, every muscle, and every internal organ has an appropriate size, shape, internal structure, and set of operating conditions. A complex set of genetic and developmental factors enable each and every bone, muscle, and organ, in any healthy animal, to: (1) continue growing until it reaches an appropriate size, shape, structure, and morphology; and then, (2) stop growing, and thereafter concern itself with maintenance, and with the gradual deaths and replacements of individual cells in a manner which keeps the bone, muscle or organ constantly renewed, healthy, and vigorous over a span of time that, in humans, is measured in decades.

Accordingly, the goal of homeostasis, within any animal, is to keep the animal's body, and all of its bones, muscles, organs, and other components, at or near the proper “setpoints”, where all of the components can function together and interact with each other in a normal and healthy manner. In a similar manner, if a bone, organ, or other tissue is injured, broken, infected, etc., then the normal response of the body is to try to repair and heal the injury, in a manner which will return the damaged component, as nearly as possible, back to its normal “setpoint”.

This leads to an important point, concerning the nature of what will happen if an NMDA antagonist is used to treat and alter a damaged nervous system, in a patient suffering from a severe neurologic problem. Instead of actively correcting the problem, the NMDA antagonist will, in effect, merely “quiet down” and stabilize the nervous system, and give it a chance to recover, in a manner that will enable the nervous system to use its own innate healing mechanisms and capability to repair itself.

By way of analogy, if a person suffers a broken arm or leg, and a plaster cast is placed around the injured limb to help it heal, the cast will never touch the broken bone, and it cannot and will not actually heal that broken bone. Instead, the role and function of the cast is merely to stabilize and protect the broken bone, and give that broken bone a period of stable calm and quiet. If a broken bone is given an opportunity to do so, it will use its own innate and internal healing capabilities, to mend and heal itself. In the same way that each and every bone, muscle, and organ in any animal or person somehow “knows” how (or is programmed by its genes) to grow to a certain size, shape, and structure, and to stop growing once it reaches its desired and targeted “setpoint”, each bone, muscle, or organ somehow retains the “knowledge” of what its particular “setpoint” is, and ought to be. Accordingly, if a bone, muscle, or organ is injured, it will try to heal and mend itself in a way which returns the injured tissue, as closely as possible, to its pre-injury setpoint. If that is not possible, it generally will attempt to at least “move in that direction”, and it will approach, as closely as possible, its original and normal size, shape, and condition.

It also should be noted that a cast should be used to protect a broken bone continuously, until the bone fully heals, before removing the cast. It does not and will not accelerate or promote the healing of a broken bone, by periodically taking off a previous cast, and then replacing it with a new cast a few days later. Indeed, the act of removing the cast and leaving it off for several days, in a misguided attempt to allow the bone to return or readjust to a more “natural” environment even though it has not yet healed, would place a broken bone in serious jeopardy of becoming reinjured.

Based on the results that have been observed in all of the continuous subanesthetic infusion tests carried out to date on patients suffering from severe neuropathic pain, it strongly appears that a damaged nervous system can repair itself, using its own innate and internal healing and mending mechanisms, in a manner that is directly analogous to the way a broken bone will mend and heal itself, if it is stabilized, protected and given some “quiet time to heal” by a cast. In either situation, a broken bone—or a damaged nervous system—must be provided with a calm, stable, and quiet period of rest, for a sufficient period of time to enable the broken bone—or damaged nervous system—to undergo a complete (or at least almost-complete) process of recovery, recuperation, and restoration.

Because of how innate healing mechanisms function in animals, it is believed and asserted by the Applicants herein that, when treating the most severe and intractable cases of chronic neurologic problems (such as neuropathic pain, depression, etc.), a continuous treatment regimen is likely to be more capable of providing effective and long-lasting relief, than an intermittent, periodic, and discontinuous treatment regimen. However, this does not rule out the development or use of lesser intermittent treatments, for milder or more moderate cases of such disorders. For example, it may be that a relatively brief out-patient treatment regimen, administered only when needed (such as every few months), may be sufficient to provide substantial relief to various cases, especially when certain specific types of neurologic disorders are involved, and the total costs of multiple intermittent treatments of that type may be substantially less than a continuous treatment regimen as disclosed herein. Accordingly, to provide a maximal margin of safety with truly minimal risks, a combination of safener drugs as disclosed herein should be seriously and carefully considered, for use in accompanying even intermittent treatment regimens of that type.

In addition, there are several factors which suggest that improved benefits might be provided, for at least some patients (and especially when treating very severe cases), if an NMDA antagonist treatment regimen can be carried out in a manner which overlays and acts consistently with a patient's normal sleep cycle, over a span of several days and night. As a general principle, the restorative powers and functions of sleep are well known, even if not fully understood. Accordingly, if a highly adverse condition within the central nervous system can be effectively quieted for a while, by using a drug regimen as disclosed herein, the additional innate healing and restorative mechanisms that will come into play, when the patient is sleeping, may be able to help the drug treatment function even more effectively.

Along those lines, a sleep-related hypothesis which was propounded by Francis Crick (the Nobel Prize-winning codiscoverer of DNA) and Graeme Mitchison should be noted, because it may be involved in the type of healing and restoration actions that can be enabled by the treatment regimens described herein. While it has not been “proven” to a level of scientific or medical consensus, it has never been disproven, and it is consistent with quite a few known facts.

Briefly, the Crick-Mitchison theory asserts that dreams provide a mammalian brain with a “house-cleaning” or “house-keeping” mechanism, which the brain uses to disconnect various synaptic connections that should not have been created. This hypothesis asserts that a dream-controlling mechanism within a mammalian brain analyzes the contents, importance, and meaning of various neuronal circuits and networks, by activating them, and then “observing” how they respond when activated. This is analogous to a detective interviewing a suspect, or a psychiatrist asking questions of a patient. Rather than trying to piece together physical evidence and then guess what a suspect (or patient) might have done or might be thinking, a more straightforward approach is to simply ask questions of the suspect (or patient), and then listen carefully to the responses. In an analogous manner, according to the Crick-Mitchison hypothesis, the brain components that control dreams wait until the brain and body are sleeping, and then use that “quiet time” to carry out functions that are analogous to what a cleaning crew does each night in a busy office building, after the daytime employees have left and gone home. By selectively turning on various neuronal circuits and networks, the brain components that control dreams are able to determine what any given set of synaptic connections will do, when activated. If the dream-controlling system somehow determines that certain synaptic connections and/or a certain neuronal circuit should not have been created or activated, then the reviewing-and-governing system apparently is capable of using some type of mechanism (which is not yet understood) to deactivate (and effectively disconnect, de-prioritize, etc.) at least some of the synaptic connections that were activated in an inappropriate and unproductive manner.

It should be noted that, under the Crick-Mitchison theory, when non-useful synaptic connections are deactivated, then the neurons which had become recruited into “active participant” status, in those non-useful networks, can be effectively disentangled, released, and “freed up”, for other uses, in other networks that will someday be needed to handle legitimate and useful memories, learning activities, etc.

To anyone who understands how mechanical or electronic systems are designed and run, and how they need to be tested, checked out, and “debugged” as a standard part of any skilled and sophisticated design and assembly process whenever a complex system is being created, the Crick-Mitchison hypothesis makes good logical sense. If a system designer or manager has a selective, useful, and “intelligent” method (and an adequate set of tools) for isolating and analyzing specific subassemblies (or “subroutines”, when computer software is involved), one at a time, within a highly complex network, to determine what role each subassembly performs (and how well it performs it), and if that designer or manager also has the ability to disconnect, reorganize, and rebuild, rewire, or recode any particular subassembly or subroutine that is not working properly, then the designer or manager will be able to create a much more efficient and workable system.

By contrast, if a mistake, problem, or “non-optimal” subassembly cannot be cleaned up, repaired, or improved, then the designers and managers of the system will need to design, create, and use an approach that computer programmers call a “work-around”. “Work-around” responses to problems are almost never as optimal or efficient as actually correcting (and thereby eliminating) a problem. By way of analogy, consider a grocery store which has a broken jar of pickles on the floor, in one particular aisle, and then consider what would happen if that mess could not be cleaned up and removed. If that were the case, the store managers would need to cordon off the problem area, using cones, warning signs, brightly-colored tape, etc.; and, customers would then have to avoid that part of the aisle, for as long as the mess (which cannot be cleaned up) remains. Clearly, that is not how grocery stores function, and for good reason. It is much more efficient to simply clean up a problem that was created by an unintended accident, and then return to normal operations.

In addition to the logic which underlies the Crick-Mitchison hypothesis, a line of evidence from comparative anatomy also supports it. Nearly all mammals “dream”, as evidenced by brain waves and “rapid eye movement” (REM) activity. However, there are a few exceptions, including the “spiny anteater” (the only mammal in the world which lays eggs, other than the platypus), and certain species of small dolphins. Each of those species has a relatively large brain, when measured by volume, but an apparently modest or even impaired level of intelligence. That is what one should expect from a system that requires a “work-around” approach, which diverts resources into inefficient modes of operation if problems are encountered, and which does not have the ability to disconnect any connections that should not have been made, in ways that would “free up” any disconnected neurons from unproductive tasks and entanglements they were mistakenly assigned or recruited into.

Additional information on various sleep-related and dream-related theories and hypotheses is available from numerous sources. Crick and Mitchison's hypothesis was originally set forth in a 1983 article; they later expanded on it and added more supporting information in a review article published in 1995. Siegel 2001 sets forth an opposing viewpoint, asserting that the hypotheses has not been adequately proven, and pointing to evidence which, in Siegel's opinion, does not appear to be consistent with the hypothesis. Other information on topics such as: (1) the relationships between “synaptic weightings”, and functions such as learning and memory; (2) the effects of REM sleep on “memory consolidation”, following learning and training sessions in behavioral tests; and (3) what is known about the “unlearning” process, in which synaptic connections are disconnected, is available in various published articles, such as Hopfield et al 1983, Horn et al 1998, and Smith et al 2004.

It must be emphasized that the validity and patentability of the invention disclosed herein does not rely on anything that is asserted or implied by the Crick-Mitchison hypothesis. Nevertheless, that hypothesis, and other known facts and prevailing beliefs about “unlearning” processes and the effects of sleep on the brain, all appear to be consistent with the exceptionally effective results that have been observed and achieved, to date, when continuous subanesthetic infusions of ketamine were used to treat patient suffering from severe neuropathic pain, in treatment regimens which lasted continuously, for multiple days and nights, while the patients slept each night and napped during the days.

Accordingly, for all of the reasons set forth above, the Applicants herein believe and assert that ketamine-plus-multiple-safener treatment regimens which last for multiple days and nights, and which will create an overlapping combination of: (i) a prolonged and continuous subanesthetic infusion of ketamine, and, (ii) a normal sleep cycle supplemented by frequent naps, will enable a damaged central nervous system to use its innate repair and healing mechanisms, to move a toward an improved homeostatic “setpoint”, in an improved manner and rate. Those effects can be especially useful, when very severe cases of the types of chronic neurologic conditions described herein are involved.

However, the apparent benefits of using a continuous and sustained treatment, lasting for multiple days and night, especially for severe cases, leads back to a crucial point of this invention. If a sustained and prolonged treatment regimen, using a potentially neurotoxic drug such as ketamine, is the best and most effective way to achieve lasting and even life-changing benefits by allowing the nervous system to effectively disconnect and “rewire” various circuits, networks, and synaptic connections, then any physicians and neurologists who are interested in this mode of treatment will need to squarely and directly address the need to accompany the ketamine with a potent safener treatment, such as the safener drug combinations disclosed herein.

The next section describes four specific NMDA antagonist drugs that cover a very wide range of potency, ranging from extremely potent, to very weak.

### Four Important NMDA Antagonist Drugs

According to conventional pharmaceutical terminology, any drug which blocks or suppresses activity at NMDA receptors is labeled and categorized as an NMDA antagonist. There are four well-known and widely studied drugs in this class. In order of decreasing potency, those four drugs are dizocilpine, PCP, ketamine, and memantine. All four of those drugs were identified and developed in a logical sequence, which was driven and guided by certain goals that arose along each step of a research pathway. Accordingly, a brief review of the history of drug development in this field can help readers understand the current state of the art.

In the 1950's, roughly 30 years before NMDA receptors had been identified and characterized (and before glutamate was even known to be a neurotransmitter), a drug called phencyclidine (PCP) was introduced into medicine as a new class of anesthetic. It was labeled a “dissociative” anesthetic, because it blocked pain signals while also rendering animal or human subjects in a trance-like state of reduced consciousness. A very desirable property of this new anesthetic was that it did not interfere with cardiac or respiratory functioning.

However, shortly after PCP was introduced into human medicine, as an anesthetic, it was abruptly withdrawn, because it was found to cause florid hallucinations, and in some patients it caused severe and sometimes prolonged psychotic reactions. Subsequently, it became a widely abused hallucinogenic street drug, as mentioned below.

After PCP was withdrawn from human medicine, researchers began looking for milder analogs of PCP which could provide similar anesthetic benefits, but in a safer and more controllable manner. Those research efforts led to ketamine, which was then developed and commercialized as a surgical anesthetic.

Because it is fully capable of rendering people totally unconscious when used in clinically relevant dosages, ketamine is classified, labeled, and referred to herein as a “potent” NMDA antagonist drug, even though it clearly is not as potent as either dizocilpine (MK-801) or phencyclidine. Currently, ketamine is approved only to provide sedation or anesthesia for relatively short periods of time, such as during surgery. Except for a few tightly limited small-scale human clinical trials, it is not approved for chronic or subchronic use over a span of days, weeks, or months.

Although the effects of ketamine are less intense than those of phencyclidine, when anesthesiologists began using ketamine on surgical patients in the 1960's, they soon realized that some patients became anxious, agitated, and sometimes hallucinatory or even overtly psychotic, as they awoke after surgery. These reactions were referred to as “ketamine emergence reactions” and were assumed to have no long-term consequences. Initially they were managed by palliative measures (such as physical restraints, calming words from trained nurses, etc.) or with tranquilizing medications. Eventually it was learned that if a sedative or tranquilizer was administered shortly before or at the same time ketamine was introduced, the frequency and severity of emergence reactions was substantially reduced. Therefore, co-administration of sedatives or tranquilizers, along with ketamine, became a standard practice among anesthesiologists.

In the 1980s, glutamate was finally recognized and proven to be an excitatory neurotransmitter. That had not been proven earlier, because of several factors. In particular, glutamate also has a completely different (and in some respects contradictory) role, as one of the 20 “primary” amino acids that are used to form all protein in all life on earth. It did not appear logical that nature and evolution would give a ubiquitous molecule, which is necessary for all protein synthesis in all cells on earth (including all neurons and glial cells in a brain and spinal cord), a crucial additional function, as a neurotransmitter, since that additional function presumably could be jeopardized and subjected to severe levels of unwanted “noise” by additional glutamate that was being synthesized and used by cells for totally different purposes.

Furthermore, no one had recognized that the glutamate transport system could perform two simultaneous but contradictory functions, as follows: (i) it must not act so rapidly and aggressively that it will grab and dispose of glutamate that has been released into a synaptic junction, before the glutamate has had time to contact and activate a neuronal receptor; however, (ii) it must act with sufficient speed and aggressiveness to allow it to clear out essentially all glutamate molecules, as soon as they have been released by a neuronal receptor, before they can contact and react a second time with a neuronal receptor.

Accordingly, when a few researchers initially suggested that glutamate appeared to be acting as a neurotransmitter, their suggestions were criticized and objected to, by other researchers, and it was not until the 1980's that glutamate was finally proven to be an actual neurotransmitter.

As soon as that was accomplished, intensive research began, to identify and characterize the types of neuronal receptors that are activated by glutamate. In studies aimed at identifying and characterizing the different types of glutamate receptors, researchers used various structural analogs of glutamate, including NMDA, and it was learned that NMDA powerfully mimics glutamate's excitatory action at some (but not all) synaptic binding sites. The NMDA-responding sites came to be known (and named) as NMDA receptors (or receptor complexes, receptor sites, etc.).

It was soon learned that the action of either NMDA or glutamate, at NMDA receptors, could be blocked by any of several drugs, including PCP and ketamine; until then, their mechanism of action simply was not known. Dizocilpine also was identified as a selective NMDA antagonist, which was even more potent than phencyclidine at blocking the excitatory activity of the natural neurotransmitter (i.e., glutamate).

In that same era, during the mid-1980s, it was discovered that excessive activation of NMDA receptors plays a major role in aggravating the extent and severity of brain damage, during a crisis where blood flow to the brain is disrupted, such as a stroke or cardiac arrest. This discovery was made by causing neurons (initially in cell culture, or in perfused slices of brain tissue) to die, by subjecting them to hypoxic and/or ischemic conditions that mimic stroke, and then demonstrating that an NMDA antagonist drug could prevent or at least reduce the extent of neuronal death that was caused by the hypoxic and/or ischemic conditions (e.g., Rothman and Olney 1986 and 1987). Since hypoxic and/or ischemic neuronal degeneration could be prevented by blocking NMDA receptors, it became clear that the neurodegenerative process which was killing the neurons was being mediated by excessive activity of glutamate, at NMDA receptors.

That discovery prompted researchers in both academia and industry to begin developing and testing NMDA antagonist drugs, as candidate drugs which hopefully could minimize the extent of brain damage, during and after a crisis such as a stroke, cardiac arrest, or near-suffocation. During that research, dizocilpine (formulated as MK-801, the maleate salt of dizocilpine) was recognized as an extremely potent and selective NMDA antagonist.

In 1989, with great hopes based on excellent results in animal tests, human clinical trials began, with dizocilpine. However, it was found that severe side effects (including hallucinations, severe agitation, etc.) occurred at doses lower than the dosages required to achieve any worthwhile benefits.

That same year, it was reported that dizocilpine (and various other NMDA antagonists, including PCP and ketamine) cause measurable neurotoxic injury, in neurons in certain portions of the brains of adult rats (Olney et al 1989). The unfavorable side effects encountered in human patients, combined with warning signals that measurable neurotoxic damage was created in animal brains following dizocilpine exposure, led to withdrawal of dizocilpine from further human clinical trials.

During the years following the withdrawal of dizocilpine from clinical trials, more data was gathered on NMDA receptors, and a number of theories and hypotheses were proposed and published, suggesting that NMDA receptor dysfunction might be causing or aggravating various types of mental illnesses and disorders that do not involve the type of acute brain damage that occurs during a stroke, cardiac arrest, or similar crisis. Since it was clear that the two highly potent NMDA antagonists (dizocilpine and phencyclidine) could not be used or even tested on humans, and since ketamine was known mainly for rendering people unconscious, attention shifted to efforts to find a relatively mild and weak NMDA antagonist which would be suited for long-term use, such as by oral ingestion of daily tablets or capsules, for treating patients suffering from chronic disorders rather than acute crises.

Those efforts to find a mild NMDA antagonist that could be taken in pill form, every day, led to memantine, a dimethyl derivative of a compound called amantadine, which has a three-dimensional structure comprising four cyclohexane rings, bonded together in a shape comparable to a pyramid or tetrahedron. Memantine was initially thought to be a selective NMDA antagonist; however, subsequent research has reported that it also appears to have both: (i) suppressing activity, at the nicotinic class of ACh receptors (e.g., Aracava et al 2005), and (ii) stimulating activity, at the muscarinic class of ACh receptors (e.g., Drever et al 2007). Because of a number of factors, memantine is discussed in more detail below, under the “Competing Schools of Thought” heading.

To complete this overview of the four major known NMDA antagonist drugs, two additional factors should be mentioned.

The first concerns a series of events in the 1990's. After NMDA receptors were identified and it was learned that PCP and ketamine suppress activity at NMDA receptors, the long-known realization that PCP can cause schizophrenia-like psychosis, in some humans who receive it, took on new significance, and researchers began to hypothesize that NMDA receptor dysfunction might be playing a role in the pathogenesis, symptoms, and/or progression of schizophrenia (e.g., Olney and Farber 1995). Therefore, researchers interested in exploring and studying that possibility began administering ketamine to normal and healthy human volunteers, in relatively low doses which were high enough to elicit a mild display of hallucinations or other psychotic symptoms, in the hope that those symptoms could be studied, in ways that might yield new insights into the mechanisms underlying schizophrenia and other forms of psychosis.

Some researchers then went beyond those types of tests on healthy volunteers, and began conducting studies in which ketamine was administered to schizophrenia patients, to determine whether it would trigger or aggravate their psychotic symptoms or episodes. The resulting reports indicated that ketamine did indeed cause schizophrenic patients to display more pronounced psychotic symptoms than they had displayed prior to ketamine administration (e.g., Lahti et al 1995). Those experiments attracted media attention, controversy, and accusations (including hostile attacks in the press and in front of Congressional committees) that those researchers were using schizophrenic patients in a manner comparable to test animals, in ways that risked harming patients who were already mentally ill (and whose ability to grant truly “informed” consent to such tests were highly questionable) without offering any significant likelihood that the subjects of those experiments might benefit in some way that could justify the risks they were being subjected to. Those accusations led to various responses, such as the creation (in February 1999) of a special review committee within the National Institute for Mental Health (NIMH), which suspended activity in a number of already-approved grants involving ketamine research, and which began a comprehensive review of the use of “high risk” human subjects in research involving neuroactive drugs. Those events are described in articles such as Marshall 1999 (available at http://www.ahrp.org/AHRPinNews/Science012299.php), and in numerous other items that can be located by an Internet search for “ketamine challenge” combined with “schizophrenia”.

In addition to those published items, off-the-record suggestions were disseminated, among the psychiatric research community, that it was extremely unlikely that any grant proposals involving any use of ketamine on schizophrenic or other mentally ill patients would ever be approved again, by the National Institutes of Health. With that as blunt guidance, it became a topic to be avoided, rather than pursued.

That episode created a lingering reluctance, on the part of neurologists, psychiatrists, and researchers, to use or even test any potent or moderately potent NMDA antagonist drugs (i.e., with a potency level comparable to ketamine, or greater) on patients suffering from mental illnesses or neurological disorders. No competent physician wants to be accused, either in public or in court, of doing potentially harmful experiments on someone who was already known to be mentally ill, or suffering from any neurological disorder that impairs brain functioning, judgment, and the ability to provide informed consent. Instead, recent research efforts have focused on attempts to create less potent NMDA antagonists, comparable to memantine but with greater selectivity and fewer interactions with other receptor types.

These efforts are exemplified by a test drug labeled as CP-101,606, developed by Pfizer, and described in articles such as Preskorn et al 2008. CP-101,606 selectively binds to one particular protein (identified as the NR2B protein) which is present in some but not all NMDA receptors. Scientists have discovered that there are four different types of NR2 proteins, in different NMDA receptors. Those four different subunit proteins are designated as NR2A, NR2B, NR2C, and NR2D, and they reportedly are expressed in different numbers and frequencies, in different parts of the brain. That presents an interesting research and treatment opportunity, and one of the goals of the research involving drugs such as CP-101,606 is to:

(i) identify drugs that will bind selectively to only one (or possibly two, but not all) of those four different subunit types;

(ii) test those drugs, in both animals and humans; and,

(iii) try to identify some drug that will create a therapeutic benefit by suppressing activity at some but not all NMDA receptors.

CP-101,606 sits within that research pathway, since it selectively binds to the “B” class of NR2 subunits, but not the A, C, or D classes. However, it reportedly causes psychotomimetic effects despite its higher level of NMDA receptor specificity.

Some day, drugs which target some but not all NMDA receptors may offer important therapeutic benefits, to certain classes of patients suffering from various neurologic disorders. However, that goal remains in the distant future, and this current application discloses a different invention.

Unless and until evidence to the contrary is gathered, it is presumed and believed that NMDA antagonist treatments, using drugs (such as ketamine) which suppress activity at all NMDA receptors, are likely to be more effective, for the purposes disclosed herein, than similar treatments using a drug which selectively targets some but not all NMDA receptors. This arises from the fact that the nature and goal of the treatments disclosed herein is to give a central nervous system which has become damaged and dysfunctional, in a patient who is suffering from a severe neurologic disorder, a period of protected, stabilized, and healing calm and quiet, so that during that quiet “healing time”, the patient's nervous system can use its own innate healing processes, to try to move closer to a healthier and more stable and functional setpoint (as described above), which the nervous system will be able to continue using, long after the drug intervention has ended.

Finally, anyone who is interested in the state of the art concerning NMDA antagonist drugs should be aware that both of the two drugs that sit in the middle of the potency spectrum (i.e., phencyclidine and ketamine) are illegally abused, in ways that generate serious concerns.

Phencyclidine has become a widely-abused hallucinogenic “street drug”, often called “angel dust”. Over the years, illegal use of PCP has caused countless drug abusers to be admitted to psychiatric hospitals with symptoms closely resembling acute schizophrenia. In addition to triggering schizophrenia-like psychotic episodes and posing a risk of brain damage in people who take it illegally, PCP has a tendency to provoke anger, aggression, and violence, especially among people who have the types of personalities, attitudes, and problems which lead some people to abuse not just relatively mild drugs, but overtly dangerous drugs as well. As a result, PCP-induced psychotic outbursts have caused numerous assaults, and quite a few unprovoked but brutal and grisly murders.

When ketamine is illegally abused, it is often referred to as “Special K”. Since it is milder than phencyclidine, it has become a “club drug”. The dangers it poses are aggravated by the fact that it is legal for human use, and therefore can be imported, distributed, and handled legally, by anyone who has what appears to be appropriate paperwork (which, presumably, can be forged and copied with relative ease).

Accordingly, despite the enormous medical potential that NMDA antagonist drugs may be able to offer, both for preventing brain damage after a medical crisis, and for treating neuropathic pain and certain other neurologic disorders, and despite the fact that hundreds of millions of dollars have been spent on research and clinical trials involving NMDA antagonist drugs, they are not being used in human medicine except in very limited situations, because of their dangerous and potentially toxic side effects.

### Neurotoxic Side Effects of NMDA Antagonists

In the late 1980's and early 1990s, after the processes and mechanisms of excitoxicity had been deciphered and described in research articles, a number of NMDA antagonist drugs were tested in various animal models of stroke, and were found to be effective in preventing acute excitotoxic neurodegeneration. However, when these drugs were entered into human clinical trials, it became evident that NMDA antagonist drugs would induce hallucinations and other psychotomimetic effects, at doses which were lower than the dosages that were required to achieve any significant neuroprotective benefits.

At about that same time, research demonstrated that the unwanted and potentially toxic side effects that were seen in animals, which were initially described as reversible if exposure to the NMDA antagonist drug was limited (Olney et al 1989), could result in irreversible cell death and permanent brain damage, if the NMDA antagonist dosage was increased or prolonged (Olney et al 1991).

Since it is necessary to understand several indicators of NMDA antagonist neurotoxicity, in order to properly understand this invention, this section briefly reviews the research that has been done in that particular field.

In the late 1980's and early 1990's, testing in animals (mainly rats) by one of the Applicants herein (Olney) revealed that either MK-801 or PCP could cause clearly evident and measurable displays and symptoms of neuronal stress and damage, in animal brains. For example, in rats, when administered intraperitoneally at doses of 0.3 mg/kg or higher (i.e., 0.3 or more milligrams of the drug, per kilogram of animal body weight), dizocilpine (in the maleate salt form known as MK-801) will create a type of neuronal stress that leads to the formation of “vacuoles” (i.e., bubbles filled with clear liquid) in cerebrocortical neurons. These references to weights or dosages of dizocilpine (such as 0.3 mg, or 0.3 mg/kg) also include the weight of the maleate salt (from maleic acid) used to create the stabilized formulation of MK-801 (dizocilpine maleate); accordingly, the toxic weights of dizocilpine itself are even lower than the “including salt” weights listed herein.

Vacuoles do not exist in healthy neurons, in a normal brain. Therefore, when vacuole formation in vulnerable regions of the brain is triggered by an NMDA antagonist drug, it indicates that the stresses which are being imposed on the brain, by that NMDA antagonist drug, are so severe that they pose a serious threat of killing neurons, especially if a dosage that will trigger vacuole formation is either exceeded, or repeated.

Of particular concern is the fact that vacuole formation, in the brains of animals that received dizocilpine (or phencyclidine or ketamine, at larger dosages) occurred most prominently in certain brain regions that can be regarded as “central switchboard” or “traffic control centers” of the brain. These “switchboard” or “traffic control” centers receive neuronal inputs from numerous parts of the brain, and are crucially important in processing and prioritizing sensory input signals, perceptions, thoughts, memories, etc., in ways that enable humans (and other animals, to lesser extents) to sort through literally millions of nerve impulses that occur every second, in ways that generate coherent and functional consciousness, memory, etc. Accordingly, the fact that the “switchboard” or “traffic control” centers of the brain are the regions that are the most vulnerable to becoming severely stressed, and in some cases permanently damaged (vernacular terms such as “fried” or “burned out” can also be applied), poses major and severe concerns about the risk of permanent brain damage.

Concerning the 0.3 mg/kg dosage of MK-801 that will cause vacuole formation in the brains of essentially all rats that are tested, three factors should be mentioned. First, that dosage is based on intraperitoneal injection. This administration route uses a needle and syringe to inject a bolus of MK-801, suspended in an inert carrier liquid, into the abdominal cavity of a rat. This is a simple, quick, and convenient mode of injection, and it is widely used in research. If desired, intravenous injection in rats can be performed by using the tail vein, but that is slower, more tedious, more difficult, and more prone to experimental error. Experience has shown that intraperitoneal injection is entirely adequate and reliable, when any of the NMDA antagonist drugs mentioned herein are being tested in rodents.

The second factor is this: it has been found that female rats will generate a more consistent (and therefore more statistically reliable and useful) vacuole response, than male rats. This runs contrary to most types of drug tests on rats, in which females tend to be avoided, because of potentially uncontrolled variables created by reproductive hormones. Most female rats that have been used, in tests done to date, are retired breeders, with ages of about 7 months or more.

The third factor is this: since the 0.3 mg/kg dosage of MK-801 is the lowest tested dosage that will consistently generate a vacuole response in essentially all female rats that are tested, it is referred to herein as an “ED₁₀₀” dosage, where “ED” refers to “effective dosage” and the “100” notation indicates that 100% of the animals that are tested will display that type of response. For convenience, to avoid having to use subscript fonts or notations, terms such as ED₁₀₀ also can be written as ED-100, or simply as ED100.

Whenever a term such as ED100 is being used, it requires an understanding of any essential factors that should be known by any researchers who might try to perform similar tests. In this situation, researchers would need to know the route of administration (intraperitoneal injection), and the types of animals used to determine the ED100 level (i.e., aging female rats).

For comparative purposes, the vacuole-forming ED100 dosage of phencyclidine is 4.0 mg/kg, which is about 10 to 15 times higher than the 0.3 mg/kg ED100 dosage of MK-801. That higher dosage level indicates that phencyclidine is only about 1/10 as potent as MK-801, in provoking vacuole formation and other indicators of severe neuronal stress and risks of permanent brain damage. The ED100 dosage of ketamine is 40 mg/kg. Accordingly, those ED100 numbers, generated using the same types of tests on the same types of animals, provide a comparative ranking of the potency of those three drugs as NMDA receptor blockers. MK-801 is the most potent NMDA receptor blocker ever discovered; it is roughly 10 times more potent than phencyclidine, which is nevertheless classified and regarded as a potent NMDA antagonist. By contrast, ketamine is regarded as having only moderate potency, at a level which is only about 1/10 the potency of phencyclidine, and about 1/100 the potency of MK-801.

If exposure to MK-801 or PCP is limited to levels which do not cause clear signs of necrosis, the neuronal stress response that is manifested and displayed by vacuole formation (and by other measurable symptoms, as described below) usually is reversible. The vacuoles will dissipate over time, and subsequent cell-staining tests will not indicate that large numbers of neurons have died. However, the absence of overt necrosis and death, among neurons, is not an adequate or reliable assurance that the neurons have not been seriously and perhaps permanently damaged or impaired. It is not possible to determine whether, and to what extent, neurons that have been stressed, to a point where they begin forming useless bubbles (filled with a clear liquid, comparable to saline solution or cerebrospinal fluid) inside their cell bodies, can recover and retain their full ability to perform in a fully functional and unimpaired mode, with no signs of lasting damage, or whether they will spend the rest of their cellular lives in an impaired condition, analogous to someone who has lost the use of his legs, or someone who has survived a serious heart attack and who will have impaired heart function for the remainder of his life.

If exposure to an NMDA antagonist drug is increased substantially beyond a single rapid injection of an ED100 dosage, neurons in the brain will begin dying, leading to permanent brain damage. Such increases in dosage and/or duration can be created in any of several ways. For example, if two ED100 dosages of a relatively long-lasting drug (such as MK-801 or PCP) are administered several hours apart from each other, or if a drug with a short half-life (such as nitrous oxide) is administered continuously at a dosage sufficient to sustain a “surgical plane of anesthesia” for a period of about 8 hours or more, neurons in the brain will die, in large numbers. Alternately, if a single injection of a potent drug such as MK-801 or PCP is administered, at a dosage more than double the ED100 dosage that causes vacuole formation in all treated animals, neurons in the brain will begin dying. If those or similar modes of heavy and/or excessive administration of an NMDA antagonist drug are used, the same types of stress and disruption that cause vacuole formation, as an early symptom and indicator of damage, will begin killing neurons in the brain or spinal cord, in ways that can be displayed and measured by using certain types of cell staining techniques, using slices of brain tissue harvested from an animal after it has been painlessly sacrificed. When neurons begin dying, brain damage becomes permanent and irreversible, for all practical purposes under the current state of the art.

If desired, various types of tests and analyses, other than vacuole formation in neurons, can be used to measure and quantify the severity of stress and neurotoxic damage that are inflicted on any particular type of test animal, by any dosage regimen of MK-801, phencyclidine, or ketamine (either in the absence or in the presence of one or more safener drugs as described below). For example, as mentioned above, cell-staining techniques can be used to distinguish between dead and living neurons, or between dead and living glial cells, in brain or spinal tissue. However, because of various factors, it takes additional hours or even days for a mortally-damaged neuron to manifest and display the biochemical indicators (such as loss of integrity of the outer cell membrane) which clearly show that a neuron has actually died and is becoming necrotic. Therefore, cell-mortality staining cannot be performed rapidly, and it is less sensitive and reliable (and more time-consuming and expensive) than simply measuring the numbers and sizes of vacuoles (i.e., empty bubbles filled with a clear liquid) in a tissue section on a microscope slide.

As another example, various known assays can be used to measure the expression levels of heat-shock proteins (or strands of mRNA that encode heat-shock proteins) in affected areas of brain tissue. “Heat shock proteins” (abbreviated as HSP's) were given that name because they were first discovered when cultured cells were briefly immersed in hot water, and then rescued. Most HSP's are involved in DNA repair, and they are generally seen as “last ditch” efforts by cells to save themselves, when subjected to potentially lethal stresses. Accordingly, assays that measure expression of HSP proteins (or messenger RNA strands that encode HSP proteins) can be used to measure and quantify neuronal stress and health. However, those types of assays require relatively expensive reagents, such as preparations of monoclonal antibodies, or complementary mRNA strands having specific mRNA sequences. In addition, such assays require additional preparation and incubation time, compared to simply using an inexpensive stain, and a conventional microscope, to evaluate the presence of vacuoles in stressed neurons.

Since vacuoles are not present in healthy neurons, the facts of cell and neuronal physiology, supported by all test data gathered to date, clearly and consistently indicate that vacuole formation, in neurons in vulnerable regions of an animal brain, offers a useful, scientifically valid, and reliable early indicator of the types of stresses that will lead to neuronal deaths and permanent brain damage, if a potent NMDA antagonist drug is administered at a dosage and/or duration which crosses and exceeds a threshold level of stress and disruption within the brain. If that threshold level of stress and disruption is exceeded, by drug-induced alterations that cause neurons within the “traffic control” portions of the brain to be bombarded by abnormally large numbers of “firing” or “depolarization” events, then those traffic control neurons, when subjected to that type of excessive bombardment, can become exhausted and depleted to a point where they will begin dying.

As used herein, the term “neurotoxic” implies that a drug can disrupt neuronal functioning to a point where neurons will begin dying, thereby leading to permanent brain damage. Accordingly, it is accurate to assert that potent NMDA antagonists such as dizocilpine and phencyclidine, and moderate (or moderately potent) NMDA antagonists such as ketamine, do indeed pose serious neurotoxic risks, and can cause neuronal death and permanent brain damage. However, as in all aspects of pharmacology and toxicology, it must be understood that any neurotoxic risks or effects of any NMDA antagonist drug will depend on the dosage and route of administration of the drug, and if prolonged or repeated administration of a drug is involved, any neurotoxic risks also will depend on the routes, duration(s), and timing of drug administration.

After Olney's findings were confirmed by other researchers, the concerns they raised about potential neurotoxicity of NMDA antagonist drugs caused the U.S. Food and Drug Administration (FDA) to develop a protocol, prescribing steps that drug companies needed to follow to characterize and quantify the neurotoxic potential of each NMDA antagonist drug for which FDA approval was being requested, for initial and small-scale human trials. Based on this information, it was determined what the maximum allowable doses would be, for each candidate drug, in human clinical trials of efficacy for treating acute crises such as stroke or head trauma.

Although a number of NMDA antagonist drugs were shown to be effective in preventing excitotoxic injury in animal tests which simulated stroke or head trauma, each such drug that reached human clinical trials was found to be ineffective in preventing excitotoxic brain damage, at the maximum dose allowed; and, at each such maximum allowable dosage, psychotomimetic and related serious adverse side effects were encountered.

Thus, the current reputation and status of NMDA antagonist drugs, as potential anti-excitotoxic neuroprotective agents, is that none of them has been shown to have a margin of safety that is sufficient to permit its use, in human therapy, at doses that can protect against excitotoxic neurodegeneration.

### Psychotomimetic Side Effects of NMDA Antagonists

As various NMDA antagonist drugs have been developed and tested in human clinical trials, it has become increasingly evident that any drug which significantly blocks the functional activity of the NMDA receptor system (at least, at a level which offers a substantial potential for treating neurologic disorders such as described herein) will produce psychotomimetic side effects.

In the early days, when these types of side effects were displayed by patients anesthetized by PCP or ketamine, it was assumed that symptoms such as hallucinations were reversible, and reasonably benign, especially when ketamine was used for anesthesia. However, animal studies showed that, while the types of neuronal stresses imposed by a reasonably brief blockade of NMDA receptors appear to be reversible, a more prolonged blockade of NMDA receptors results in neuronal deaths, and permanent brain damage. Accordingly, researchers and regulatory authorities have been obliged to give very serious consideration to the evidence which indicates that: (i) psychotomimetic symptoms appear to function as warning signals, indicating that neurons are being placed under serious and possibly severe stress, whenever NMDA receptors are being blockaded at levels which begin triggering hallucinations and other psychotomimetic symptoms; and, (ii) if such stresses are prolonged, the affected neurons can be stressed so severely that they will die.

Furthermore, as described below, and as part of this invention, the Applicants herein have discovered and established a strong correlation between:

(i) the rankings of various candidate drugs, when measured and quantified by their ability to prevent vacuoles from forming in vulnerable portions of the brains of test animals; and,

(ii) the rankings of those same candidate drugs, when measured and quantified by their ability to suppress hallucinations and other psychotomimetic effects, in healthy human volunteers who have been given dosages of ketamine that otherwise would be sufficient to trigger psychotomimetic effects.

Accordingly, these new findings both:

(1) add additional evidence which further supports the assertion and conclusion that psychotomimetic symptoms (such as hallucinations) are indeed valid and critical warning signals, which indicate that permanent brain damage will indeed occur, in humans as well as test animals, if an NMDA antagonist drug dosage which is sufficient to trigger such psychotomimetic symptoms is continued for a prolonged period of time; and,

(2) provide a powerful and useful research tool, for measuring and optimizing the efficacy of various combinations of safener drugs that will be useful for different types of prolonged medical treatments, as described herein.

In summary, all available data indicate that there are strong correlations between: (i) the psychotomimetic symptoms caused by NMDA antagonist drugs, and (ii) the threat that permanent and serious brain damage will occur, if NMDA antagonist drugs are administered to patients suffering from unwanted NR/hyper activity, for prolonged periods of time and at dosages sufficient to achieve lasting therapeutic benefits. These data, along with various other concerns as described above, have posed major and severe obstacles to the use of NMDA antagonist drugs for treating neurologic or psychiatric disorders. Although limited and small-scale clinical trials are being done by scattered teams of researchers, commercial drug development and industry-supported research, in this particular field, apparently are at a standstill, and are not moving forward in any way which addresses or reflects the huge potential of such drugs to relieve suffering, and to treat or prevent a number of extremely severe medical problems.

Drugs that Suppress Activity at ACh and Non-NMDA Receptors

Since two categories of drugs, in particular, are of major interest herein, this section provides a brief introduction to those two classes of drugs.

The first class includes drugs that suppress activity at neuronal receptors that are activated by acetylcholine (ACh). This class of drugs (usually called anti-cholinergic drugs) has been known for decades, and a wide assortment of such drugs is well-known and available. Any recent edition of Goodman and Gilman's Pharmacologic Basis of Therapeutics (the most widely respected textbook on pharmaceuticals, initially published in 1941 and currently in its 11^{th} edition) has an entire chapter devoted to drugs that stimulate or suppress the “muscarinic” class of ACh receptors (the other main class of ACh receptors is nicotinic receptors).

The only recent development that merits particular attention, concerning anti-cholinergic drugs, is the discovery of a class of so-called “long-acting muscarinic antagonist” (LAMA) drugs, which are reviewed in articles such as Casarosa et al 2009 and Joos 2010. These drugs include tiotropium bromide, an inhalable formulation sold under the trademark SPIRIVA, used by people who suffer from chronic obstructive pulmonary disease (COPD). It requires inhalation only once per day, to help control COPD. Other LAMA drugs which have reached clinical investigation include aclidinium bromide, and glycopyrrolate (which, however, does not cross the blood-brain barrier in quantities sufficient to render it of interest for use herein). While it may be convenient to use a long-acting anti-cholinergic drug as a safener drug for use as disclosed herein, it is not essential. Since a preferred mode for administering a safener-plus-ketamine combination (at least for treating very serious disorders) normally will involve continuous intravenous infusion of at least the ketamine component, for several days in succession, in a hospital or clinic where a patient's vital signs can be continuously monitored, any effective anti-cholinergic drug that is selected for such use, in any particular patient, can be coadministered (either orally or intravenously) with the ketamine at any desired frequency (if administered orally) or infusion rate (if administered intravenously).

A more complex set of prior art has emerged over the past fifteen years, involving a number of drugs discovered just recently, which can selectively suppress activity at non-NMDA receptors (i.e., at the KA and/or AMPA classes of glutamate receptors), without also suppressing activity at NMDA receptors. These “non-NMDA antagonists” arose mainly from the discovery that a compound called “willardiine” will activate the KA and AMPA classes of glutamate receptors, without also activating NMDA receptors. Willardiine is the common name for 3-(2,4-dioxo-3,4-dihydropyrimidin-1(2H)-yl)-L-alanine, which has two oxygen molecules, and an alanine moiety, bonded to a pyrimidine ring, in a certain configuration.

After it was discovered that willardiine will selectively activate KA and AMPA receptors but not NMDA receptors, researchers began testing analogs and derivatives of willardiine, in an effort to identify altered versions of willardiine which have the opposite effects, and which suppress (rather than increase) activity at KA and/or AMPA receptors. This line of research is described in articles such as More et al 2003.

Most receptor-suppressing activity by KA and/or AMPA antagonist drugs is usually attributed to “competitive binding” mechanisms, which can involve either of the following mechanisms:

(i) some antagonist drugs will bind to KA and/or AMPA receptors without triggering the activation of those receptors; in this manner, they will block and prevent the natural neurotransmitter (i.e., glutamate) from reaching and activating receptors which are occupied by the antagonist drug; or,

(ii) other antagonist drugs will trigger a single “firing” event when they bind to a KA and/or AMPA receptor, but these drugs will then remain bound to the KA and/or AMPA receptors for abnormally long periods of time, in ways that will prevent and suppress subsequent firing events.

It also should be noted that some non-NMDA antagonist drugs will bind, not to the actual and specific binding site where a molecule of glutamate contacts and binds to a KA or AMPA receptor, but to a different region of a receptor protein or complex, in a manner which effectively clutters or entangles the receptor, and impedes the ability of glutamate to reach its binding site. This type of binding reaction can be regarded as a competitive binding reaction, since the drug will effectively compete against glutamate, for binding to the overall receptor. However, experts who specialize in this type of research, and who want to be able to further distinguish between various different molecular mechanisms, usually refer to that type of binding reaction as “non-competitive” binding, or as “allosteric” modulation or inhibition of a receptor.

Any of these types of KA and/or AMPA receptor-suppressing effects can be demonstrated, measured, and quantified, using relatively simple and inexpensive “in vitro” tests that involve cell or tissue cultures. A number of candidate drugs which showed good efficacy, in in vitro tests, have moved to the next stage of research, which involves testing in small animals, such as mice or rats. Because the nature and purpose of these drugs is to slow down neuronal activity, the most common types of animal tests that are used to evaluate the effects of these drugs, in live animals, utilize “models” or “simulations” which will evaluate whether a candidate drug can effectively suppress and control seizures, or convulsions. A number of such animal tests are known, which typically involve any or all of: (i) a mild dosage of a “challenge” drug which will provoke seizures or convulsions in the absence of a protective drug; (ii) electrodes that have been implanted in the brain, which can be used to deliver currents and voltages that will trigger seizures or convulsions; and/or, (iii) specially-bred strains of mice or rats that have “knockout” gene defects or other factors that cause abnormally high numbers of seizure events.

Since epilepsy is the most common neurologic disorder that involves seizures and convulsions, the types of drugs that can suppress and prevent seizures and convulsions are usually referred to, in the scientific and medical literature, as anti-epileptic drugs (AED's). Therefore, a large body of literature on drugs that have been found to suppress activity at non-NMDA receptors can be found quickly and easily, by searching the National Library of Medicine database for the terms AMPA, kainate, or glutamate, combined with the terms epileptic or anti-epileptic.

Accordingly, drugs that selectively suppress KA receptor activity include ACET, LY382884, ATPA, and 5-iodowillardiine (reviewed in articles such as Jane et al 2009), LU 97175 (e.g., Loscher et al 1999), and certain thieno-oxazine compounds (e.g., Briel et al 2009).

Drugs that selectively suppress AMPA receptor activity include NS1209 (e.g., Blackburn-Munro et al 2004), GYKI-52466 (e.g., Lees 2000), UBP277 and UBP282 (e.g., Ahmed 2009), LU 112313 (e.g., Loscher et al 1999), and talampanel (e.g., Rogawski 2006).

Drugs that suppress activity at both KA and AMPA receptors, without also suppressing activity at NMDA receptors, include topiramate (e.g., Rosenfeld 1997 and Nakamura et al 2000), and LU 115455 and LU 136541 (e.g., Loscher et al 1999). It should also be noted that topiramate reportedly also has activity at neuronal sodium channels, and may also potentiate the effects of GABA.

It should also be noted that several drugs have been discovered which reportedly suppress activity at all three classes of glutamate receptors (i.e., NMDA receptors, KA receptors, and AMPA receptors). Such drugs include LU 73068 (e.g., Potschka et al 1998).

Research into drugs that can help reduce and control seizures, convulsions, and other manifestations of unwanted excitation, inside the brain, has expanded dramatically within the past 15 years, and numerous review articles (including Patsalos 2005, Perucca et al 1996, Luszczki 1999, Rogawski 2006, and Johannessen-Landmark et al 2010, as just a few examples) describe the cellular mechanisms of numerous such drugs. Briefly, these drugs fall into any of several different classes, depending on which particular types of neuronal receptors and/or ion channels they interact with. Three of the most common mechanisms used by these types of drugs can be briefly summarized as following.

One major class of such drugs, generally referred to as “GABAergic” drugs, interact with GABA receptors. GABA refers to gamma-amino-butyric acid, a naturally-occurring substance that is one of the most important inhibitory neurotransmitters. Most GABA receptors are located along the shafts of neuronal fibers, rather than at their tips. When a molecule of GABA contacts and binds to a GABA receptor somewhere along the length of a neuronal fiber, it reduces the tendency and/or ability of the affected nerve fiber to convey an electrochemical impulse, either toward the neuronal body, or toward the neuronal tip. In that manner, GABA decreases the quantities of ACh and glutamate that are released by neurons in response to neuronal “firing” or “depolarization” events.

A second major class of such drugs, generally referred to as “channel blocker” drugs, uses binding reactions which create partial blockages of certain types of ion channels associated with synaptic receptor complexes. At the current time, it appears that the most important drugs in this category which have emerged during the past 20 years target either sodium channels, or calcium channels. Under normal conditions, sodium or calcium channels are briefly opened, when a nerve impulse is transmitted through a neuronal fiber. The opening of these ion channels, which pass through the neuronal membranes, allows positively-charged Na⁺ or Ca⁺⁺ ions to flow through the ion channels, until a “voltage gradient” (which exists across the neuronal membrane) drops from a “resting state” (usually about 90 millivolts, for most neurons), to a “channel closing voltage” (usually about 65 millivolts). Therefore, drug molecules which bind to specific protein sites that are accessible, as structural components of these types of ion channels, will hinder and slow down the flow of ions through these ion channels. In this manner, “channel blocker” drugs tend to suppress and reduce neuronal activity.

Two “channel blocker” drugs in particular deserve special attention, because, together, they created a major medical advance. They are gabapentin (approved by the FDA in 1993, and sold under the trademark NEURONTIN), and pregabalin (approved in early 2004, and sold under the trademark LYRICA). Their names tend to suggest that they likely bind to or otherwise involve GABA receptors, but that is not the case; instead, they were initially developed and analyzed as analogs of GABA, with the assumption and expectation that they likely would affect GABA receptors, but they were later discovered to exert their primary effects by actions that apparently involve binding to a specific protein subunit of a certain class of calcium channels. This places them in the category of “channel blocker” drugs, as that term is defined and used herein. It should be noted that pregabalin is sometimes called a “developmental successor” to gabapentin; this arises from the fact that the therapeutic benefits and the commercial success of the earlier drug, gabapentin, motivated a number of pharmaceutical companies to begin creating and studying analogs, derivatives, and variants of gabapentin. It also should be noted that the initial FDA approval for pregabalin (LYRICA), in early 2004, was limited to treatments for epilepsy. However, it was shown to offer important benefits in treating both post-surgical pain, and certain types of neuropathic pain (including fibromyalgia), so its FDA approval was substantially expanded in 2009, and it is now being marketed for those types of pain control.

The third major class of newly-emergent candidate drugs which are currently classified as anti-epileptic drugs includes agents which suppress activity at non-NMDA receptors (i.e., KA and/or AMPA receptors), as discussed above.

As mentioned above, most drugs that slow down activity levels within the CNS are usually classified, at least during the initial stages of research, as anti-epileptic drugs. Therefore, anyone who is surveying or studying the field of known and recently-discovered anti-epileptic drug candidates will encounter frequent references to “second generation” and “third generation” drugs. To interpret those terms, it is helpful to know that prior to about 1990, four specific drugs (phenyloin, carbamazepine, valproic acid, and phenobarbital) formed a “baseline” set of anti-epileptic drugs, which provided the major treatment options in 1990. Drugs that emerged after 1990, until about 2004 (when pregabalin was approved for treating epilepsy) are classified as “second generation” drugs. Drugs that have emerged after pregabalin became a major option for treating epilepsy are called “third generation” drugs.

Although there are some discrepancies between different review articles that describe “second generation” AED's, they typically include, in addition to gabapentin and pregabalin, drugs such as felbamate, lamotrigine, levetiracetam, oxcarbazepine, rufinamide, stiripentol, tiagabine, topiramate, vigabatrin, and zonisamide.

Other drugs which usually are labeled as “third-generation” anti-epileptic drugs include, for example, levetiracetam analogs such as brivaracetam and seletracetam; a number of valproate-like agents, including valrocemide, valnoctamide, propylisopropyl acetamide, and isovaleramide; a felbamate analog called fluorofelbamate; a number of oxcarbazepine analogs, such as licarbazepine, eslicarbazepine, and BIA 2-093; several benzodiazepine receptor agonists, including ELB139; and, an assortment of other candidate drugs, such as lacosamide, and carbamate RWJ-333369.

It should also be noted that a number of drug candidates have been identified, which appear to act via two or more different mechanisms and/or receptor types. Examples include talampanel, which is believed to act at benzodiazepine receptors as well as AMPA receptors; ganaxolone, which reportedly acts at GABA(A) receptors as well as neuroactive steroid receptors; and retigabine, which reportedly acts at potassium channels as well as GABA(A) receptors.

Accordingly, an assortment of known drugs has emerged, during the past 20 years, which can help suppress activity at either or both of the KA and AMPA classes of glutamate receptors. As described below, any such drug which is known to suppress activity at KA and/or AMPA receptors can be tested and evaluated, using no more than routine experimentation, to evaluate its suitability and potency as a “safener drug” for preventing the neurotoxic side effects of prolonged administration of ketamine (or any other selected NMDA antagonist drug) for a medical purpose as described herein.

This completes an overview of the prior art which, in the opinions of most experts in this field, would qualify as valid, proven, and known science. As mentioned at the beginning of this Background section, the next two subsections shift into a different mode of explanation and analysis. Rather than describing widely-accepted and agreed-upon scientific conclusions, they attempt to summarize several theories and hypotheses that have not yet been proven, and that are competing against each other for attention, respect, research funding, and research commitments.

For reasons described in the subsection immediately below, which describes “safener drugs”, this cluster of information sits at a halfway point, somewhere between “accepted truth” and “mere hypothesis”, without fully or cleanly belonging in either category. Very briefly, the data described have largely been generated in animal studies and are sound and reliable; however, for reasons of their own, a number of scientists have challenged their applicability and relevance in a human context. As a result, this information sits in the transition zone, between “proven truth” and “mere hypothesis”.

### Safener Drugs

In various published articles and issued patents, one of the Applicants herein (Olney) has previously disclosed that, in tests using rats, several different categories of known drugs can reduce and in some cases prevent the neurotoxic side effects of potent NMDA antagonist drugs such as MK-801 or phencyclidine. Such drugs include:

(1) Anticholinergic drugs which block the muscarinic class of cholinergic receptors. Examples include scopolamine, atropine, benztropine, trihexyphenidyl, biperiden, procyclidine, benactyzine, and diphenhydramine. The safening activity of this class of drugs is described in more detail in Olney et al 1991, and in U.S. Pat. No. 5,034,400 (Olney 1991).

(2) Benzodiazepine drugs which can increase (“potentiate”) the effects of a naturally-occurring inhibitory neurotransmitter called gamma-amino-butyric acid (GABA) have some efficacy in preventing toxic side effects of NMDA antagonists. However, even in very high dosages, these drugs are not completely effective; they can only act to enhance the effects of naturally-occurring GABA, and become ineffective when adequate supplies of naturally-occurring GABA are not present. Examples include diazepam, which is sold under the trademark VALIUM, and its various analogs.

(3) Other drugs which can act directly at GABA type A (GABA_{A}) receptors, to open the chloride ion channel even in the absence of naturally occurring GABA, can block the toxic side effects of NMDA antagonists much more effectively than benzodiazepine drugs (see U.S. Pat. No. 5,474,990, Olney 1995). This class of drugs, called “direct GABA agonists”, includes certain barbiturates such as secobarbital and pentobarbital. It also includes certain anesthetics such as isoflurane and halothane, which are administered by inhalation, as well as propofol, an intravenous anesthetic. In addition, certain agents known as steroid anesthetics or neurosteroids, such as alfaxolone and ganaxolone, are also included within the class of “direct GABA agonists”.

(4) Drugs that can bind to a class of receptors called sigma receptors, as described in Farber et al 1993. These drugs include di(2tolyl)guanidine and rimcazole, which are relatively selective for sigma receptors. Haloperidol, which interacts with dopamine receptors as well as sigma receptors, was not as potent or effective, and required substantially higher dosages to exert significant safening activity. The results from haloperidol, and similar results indicating only low levels of safening activity in tests on a number of dopaminergic agents, tend to suggest that drugs with substantial activity at dopamine receptors should generally be avoided, at least during the early stages of testing potential safener combinations as disclosed herein.

(5) Drugs that act as agonists at a class of receptors called alpha-2 adrenergic receptors. Drugs in this class include clonidine, p-iodoclonidine, guanabenz, guanfacine, dexmeditomidine, xylazine, and lofexidine (Farber et al 1995a and 1995b).

(6) Certain types of drugs which are known to act at multiple receptor types, and which are often referred to as “atypical” antipsychotic agents, have also been shown, in tests on rats, to reduce the toxic side effects of MK-801 and PCP. Such drugs include clozapine (Farber et al 1993), olanzapine, and fluperlapine (Farber et al 1996) In addition to acting at dopamine, serotonin, and norepinephrine receptors, these drugs also bind to and suppress activity at muscarinic receptors. Accordingly, these drugs can be grouped together with the anti-cholinergic (muscarinic antagonist) drugs mentioned in item #1, above.

(7) Drugs that act as agonists at a specific type of serotonin receptor, designated as the 5HT-2A receptor, can reduce the toxic side effects of NMDA antagonists. It should be noted that drugs which agonize both 5HT-2A receptors and 5HT-2C receptors are usually hallucinogenic, and should be avoided. One such drug which agonizes 5HT-2A receptors, and which also is believed to have some blocking (antagonizing) effect at 5HT-2C receptors, is lisuride. It does not cause hallucinations, and is widely used in Europe for several purposes, such as treating migraine headaches, and helping women stop lactating when it is time to stop nursing a baby. Lisuride is also used to treat Parkinson's disease, since it is a dopamine agonist. In light of various test results, and in light of the fact that the serotonin class of receptors is exceptionally complex and plays important roles in various moods and emotions, serotonin receptors are not regarded as promising target candidates for the type of safening activity of interest herein, especially when compared to muscarinic ACh and non-NMDA glutamate receptors.

It must be recognized and understood that:

(1) the foregoing list of candidate “safener drugs” was developed through testing of rats, rather than humans, monkeys, or other primates; and,

(2) the identification of these drugs as “safener drugs”, by one of the Applicants herein (Olney), has not led to any commercialization, of any sort, as agents for accompanying an NMDA antagonist treatment regimen, to make the NMDA antagonist treatment safer. There simply has been no commercial or pharmaceutical industry effort or commitment to use any of these types of drugs, as “safener drugs”. Instead, the pharmaceutical industry has chosen to treat and regard these findings, in rat tests, as having no “proven relevance” in humans, or in primates. If any primate tests (using monkeys) have been done in any pharmaceutical company labs, the results have been kept strictly confidential, and have never been published or otherwise disclosed.

This exact same issue (i.e., whether data from rat tests is relevant, useful, and helpful, in assessing potentially toxic threats to human brains created by NMDA antagonist drugs) was also raised, in a different context, by other work by one of the Applicants herein (Olney), and it is revealing to see how that inquiry played out. Briefly, Olney and his coworkers reported (in articles such as Jevtovic-Todorovic et al 2003, and Olney et al 2004a and 2004b) that anesthesia of immature brains, using NMDA antagonist drugs (or GABA agonists) could artificially trigger a natural process that occurs in immature brains, called “neuroapoptosis”. In nature, the brains of mammals are initially created with more neurons than are needed, and the developmental process of “neuroapoptosis” is used to kill and remove neurons that have not become properly connected into the active neuronal networks of the brain (this process is analogous to the pruning of plants and trees). Tests in rats, by Olney and his coworkers, indicated that if a fetus or neonate is anesthetized (as might be required, for example, for surgery to repair a congenital heart defect or other problem), then the drug-induced temporary inactivation of the neurons, in the infant brain, could lead to a toxic and unwanted form of neuroapoptosis which could lead to permanent brain damage.

However, when those data were published, they provoked defensive and even combative replies by a number of anesthesiologists, who apparently were offended by any suggestion that the practices they were using might be causing brain damage, in babies. Their rebuttals and criticisms of Olney's work and publications is contained in articles such as Anand and Soriano 2004, Soriano et al 2005, McClaine et al 2005, and Bartels et al 2009.

However, the exchanges did not end with those criticisms. Because of the clear and undeniable importance of the questions raised by Olney's published articles, other scientists began to study the issue, and over a period of several years, multiple independent laboratories confirmed the initial findings in rodent studies, and reported that immature primates are also susceptible to the same types of anesthesia-triggered brain damage that was previously reported in rats. The critics began to back down, but still asserted that there was no evidence from human research suggesting that the developing human brain is susceptible to anesthesia-induced neuroapoptosis. That lack of evidence did not prove Olney's findings wrong; instead, it simply indicated that the issue had not been adequately studied or addressed. Therefore, epidemiological studies were undertaken by several research groups, which then began to report findings (e.g., Wilder et al 2009, Kalkman et al 2009, DiMaggio et al 2009, and Thomas et al 2010) which indicated that anesthesia during infancy, such as for surgery, was statistically correlated with significantly increased risk for learning impairment and cognitive delays that could be detected as those children grew older. Accordingly, the tide of opinion among experts, in that debate, has recently shifted to a position that can be described as “focused concern”. Critics are no longer claiming that these concerns have not yet been proven to a point that would justify or require a change in practice; instead, serious efforts are now being undertaken by anesthesiologists, pediatric neurologists, and public health officials to identify steps and precautions that will avoid or minimize any brain damage that might be caused by exposure of fetuses or infants to surgical anesthesia.

In view of the number of surgeries that are done on neonates and infants each year, it is regrettable that it took roughly five years for researchers to establish enough data to convince anesthesiologists that: (i) their practices needed to be changed and updated, and (ii) extra precautions to avoid brain damage are required, when fetuses and infants must be exposed to anesthesia. However, that is the nature of scientific and medical research, and that series of published exchanges, over a span of multiple years, offers a good illustration of a basic principle which clearly applies to neuropharmacology. After a research finding has been published, even by a respected researcher from a respected institution, it may require many years, and multiple concordant publications by numerous different research teams that have no connections to each other, before “experts” in the field are willing to accept it as an established scientific and/or medical truth.

### Competing Schools of Thought

Anyone interested in analyzing the published art, in the field of neuropharmacology research, needs to recognize and understand that quite a number of published articles in this field do not represent scientific “knowledge”, but instead propose and suggest theories and hypotheses that have not yet been “proven” to any level of consensus among experts in the field.

The true state of the art, in this field of neuropharmacology, is poignantly and powerfully illustrated by the inability of even the most skilled physicians and neurologists of modern times to cure, or even relieve the symptoms of, the most serious neurological disorders that plague humanity. Despite everything physicians and researchers have learned about the brain through centuries of study, there still are no adequate methods for preventing, curing, or reversing any of the most important disorders of the nervous system, including stroke, Alzheimer's disease, Parkinson's disease, Huntington's disease, amyotrophic lateral sclerosis, schizophrenia, severe depression, neuropathic pain, post-traumatic stress disorders, etc.

Accordingly, since this technical field focuses on extraordinarily difficult and intractable disorders of the nervous system that have defied all efforts to find cures, anyone who wishes to truly understand the state of the art, in this field of science and medicine, must be able to distinguish between what is actually known, versus what has merely been proposed and suggested as a theory, hypothesis, and hope.

### The Olney-Farber “Four Sequential Classes of Neurons” Theory

The first example which will be offered, of a theory and hypothesis which has not yet been proven or generally accepted by experts, has been previously published by two of the Applicants herein (John Olney and Nuri Farber), in a series of US patents that relate to treatments for Alzheimer's disease. These patents include U.S. Pat. No. 5,877,173 (Olney and Farber 1999), U.S. Pat. No. 5,958,919 (Olney and Farber 1999), and U.S. Pat. No. 6,391,871 (Olney and Farber 2002). All three of those patents contain the same FIGS. 1 and 2 that are included herein, and the following description of FIGS. 1 and 2 essentially repeats the same analysis of those figures that was contained in those patents, first issued more than 10 years ago.

It is crucially important to realize that the teachings in those patents never led any drug company to invest in any clinical trials, of any sort, or even in any animal research that was published in a refereed journal, to determine whether this theory is either correct, or incorrect. Indeed, the exact opposite has happened, in real life. The two major drugs that have been approved by the FDA for treating Alzheimer's disease, since the Olney & Farber patents were issued, use and rely upon mechanisms which are directly contradictory to the theories and teachings in the above-cited patents. And yet, all of the data gathered by Olney and Farber, over more than a decade since those patents were issued, have continued to add more support, credibility, and confirmation to the theory and analysis he published more than 10 years ago.

Accordingly, a direct comparison of the Olney-Farber analysis, interpretation, and theory of what is happening inside the brains of people suffering from Alzheimer's disease, against the mechanisms that are exploited by the two major drugs that have been approved by the FDA for treating Alzheimer's disease, can offer a clear and direct example of how published theories, analyses, and hypotheses do not constitute “knowledge” in this area of neuropharmacology.

Referring to the drawings, FIGS. 1 and 2 depict essentially the same things, in two different ways. FIG. 1 is a schematic outline of an animal brain, with the forebrain on the left, and the brain stem toward the lower right. Four different classes of numbered neurons are illustrated, within that brain outline. Those same four classes of neurons are also illustrated in a descending “flow chart” manner, in FIG. 2, to highlight and emphasize the sequential nature of their effects on each other.

The following terms are used to describe these four different classes or stages of interacting neurons: (1) glutamate control neurons, represented by a single bold-outline neuron 10 in FIG. 1; (2) inhibitor neurons, represented by neurons 20-50 in FIGS. 1 and 2; (3) exciter neurons, represented by neurons 20-50 in FIGS. 1 and 2; and (4) “target” neurons, represented by a single pyramidal neuron 100, in FIGS. 1 and 2. The neurons in each successive stage release neurotransmitters which contact, and either stimulate or inhibit, the neurons in the subsequent stage.

At the start of the circuit that is shown, in alternate ways, in both of FIGS. 1 and 2 is a “glutamate control neuron” 10, which releases glutamate in tiny amounts, but on a continuous or nearly continuous basis. The glutamate is released at an array of synapses that emerge from glutamate control neuron 10.

The glutamate which is released at the synapses of glutamate control neuron contacts and activates NMDA receptors on the surfaces of the second stage neurons, which are labeled as “inhibitor neurons” 20, 30, and 40. Three of those inhibitor neurons, numbered as 20, 30, and 40, are called “GABAergic” neurons, since they release GABA (gamma amino butyric acid, an inhibitory neurotransmitter) when the GABAergic neurons are activated by glutamate acting at their NMDA receptors. Other inhibitory neurons, which release other transmitters such as serotonin or norepinephrine, are discussed below.

The slow, steady release of GLU by glutamate control neuron 10 provides a steady, continuous driving force that keeps inhibitor neurons 20-40 in a constant state of activity, resulting in continuous (or nearly continuous) release of GABA.

The GABA which has been released by the inhibitor neurons 20-40 contacts GABA receptors on the surfaces of “exciter neurons” 60, 70, and 80, each of which releases a different type of excitatory transmitter. Exciter neuron 60 releases neuropeptide Y (NPY); exciter neuron 70 releases glutamate; and exciter neuron 80 releases acetylcholine (ACh).

All three of the exciter neurons 60, 70, and 80 are coupled, via axons, to a pyramidal “target neuron” 100, located in the posterior cingulate or retrosplenial (PC/RS) cortex of the brain. If all three of the excitatory neurons 60, 70, and 80 begin firing simultaneously, they can overstimulate pyramidal neuron 100, and push it to a point where it becomes so exhausted that it begins to suffer serious damage and eventually dies from overstimulation. Accordingly, pyramidal neuron 100 is labeled herein as a “target neuron”, since it can be destroyed if hit by too many “bullets” released by the exciter neurons. As discussed below, pyramidal neurons in the PC/RS cortex are merely one type of target neuron; they are highly vulnerable and consistently damaged, when NMDA antagonists are administered to lab animals, so they are usually studied and analyzed as a highly sensitive indicator of excitotoxic damage. Other types of neurons in other regions of the brain also suffer similar damage (albeit, usually to a lesser degree), and are also target neurons.

The pyramidal target neuron 100 is shown as having three different types of excitatory receptors: (1) kainic acid receptors, which are a type of non-NMDA glutamate receptor; (2) m3 receptors, which are a type of acetylcholine muscarinic receptor; and, (3) sigma receptors, which are believed to be triggered by neuropeptide Y (NPY). The presence of all three types of excitatory receptors, on pyramidal neurons, is supported by experimental evidence, showing that if activity at any one of these three classes of receptors is blocked, then any damage to pyramidal neurons, by NMDA antagonist drugs, is substantially reduced or prevented.

Thus, glutamate stimulates various GABAergic neurons to release GABA, an inhibitory transmitter. This glutamate-driven slow, steady release of GABA establishes a condition which neurologists call “tonic inhibition”, which helps restrain and limit the activity of several downstream excitatory paths. The phrase “tonic inhibition” implies that this mechanism maintains an inhibitory tone in the system.

This type of tonic inhibition represents an important principle (and an apparent paradox) of CNS activity. An excitatory neurotransmitter, such as glutamate, can cause suppression, rather than excitation, of neuronal activity. This is important for sustaining various cellular functions in the CNS; and, malfunctions in this system lead to a problem called “disinhibition”, which can contribute to dysfunction and degeneration of neurons in the brain of an animal or human suffering from NMDA receptor hypofunction (NR/hypo).

Specifically, if the NMDA receptors which govern GABAergic neurons 20, 30, and 40 (and norepinephrine neuron 50), as shown in FIGS. 1 and 2, are blocked by an NMDA antagonist drug such as phencyclidine or ketamine, or if they are rendered hypofunctional by a disease process which has left the NMDA receptors on neurons 20-40 “burned out”, then the ability of glutamate control neuron 10 to tonically inhibit the downstream “exciter” neurons 60, 70, and 80 (via GABAergic neurons 20, 30, and 40) is lost. This loss of glutamate-mediated control is referred to herein as “disinhibition”, since it interferes with the inhibitory mechanism that normally protects pyramidal neuron 100. When disinhibition occurs due to NMDA receptor hypofunction, all three excitatory neurons 60, 70, and 80 can begin to overstimulate pyramidal neuron 100, and may push it to a point where it becomes so exhausted that it suffers serious damage and eventually dies from overstimulation.

In summary, if NMDA receptor hypo-function (NR/hypo) affects inhibitory neurons 20-40, it can endanger and lead to the deaths of various target neurons, such as pyramidal neuron 100.

The schematic diagram in FIG. 1 also depicts an inhibitory neuron 50 located in the brain stem. When stimulated by glutamate, neuron 50 will normally secrete norepinephrine (NE) into the forebrain, via long fibrous processes. This is an additional regulatory mechanism for controlling the release of acetylcholine (ACh) from neuron 80. If the NMDA receptor on the NE-releasing neuron 50 is blocked or suppressed, so that glutamate cannot drive the inhibitory neuron 50 and cause it to release NE, its inhibitory action is turned off (disinhibited). This releases the ACh neuron 80 from its inhibitory control mechanism.

The simplified depiction in FIG. 1 indicates that a single glutamate-releasing neuron 10 interacts with all four inhibitory neurons 20, 30, 40, and 50. This depiction is used merely to avoid clutter in the drawing. In the brain of any mammal, thousands or millions of glutamate-releasing neurons will interact, to sustain tonic inhibition of neuronal circuits involving thousands or millions of inhibitory neurons.

Similarly, the single target neuron 100, shown in FIGS. 1 and 2, represents thousands or millions of neurons which are placed at risk, when NMDA receptors no longer function at normal and proper levels. Those endangered neurons are scattered widely throughout a number of corticolimbic regions of the brain. The PC/RS cortical region has been the focal point of the examinations described in the Examples, because it is one of the most heavily damaged portions of the brain, and one of the most consistent and reliable areas for measuring and quantifying damage. This is not meant to imply that it is the only area damaged or at risk; on the contrary, many other neurons in many other brain regions are also at risk.

To the best of the Applicants' knowledge and belief, that analysis, which arose from extensive data from animal testing over a span of numerous years, is entirely correct and accurate. However, there are different and potentially conflicting schools of thought regarding the functional, toxicological, and medical consequences that can arise when activity at NMDA receptors is suppressed by drugs having various different glutamate-blocking potencies, in patients with various medical conditions that require treatment. Therefore, it is worthwhile to compare the Olney-Farber hypothesis against a theory promulgated by certain researchers who worked to obtain government approval to market memantine, a “mild” NMDA antagonist drug, for treating people suffering from Alzheimer's disease.

Memantine is the only NMDA antagonist drug that has ever been knowingly approved by the FDA (i.e., with the knowledge that it is an NMDA antagonist; in other words, it is the only NMDA antagonist that has been approved by the FDA, for public sale and use, ever since NMDA receptors were discovered, in about 1980). Since memantine is the only NMDA antagonist in that class, it merits extra attention and careful analysis by anyone who wishes to survey the state of the art in NMDA antagonist drugs.

Furthermore, since memantine was approved, by the FDA, for only a tightly constrained and limited use (i.e., for treating only those patients who have already passed beyond the early stages of Alzheimer's disease, and have already reached an irreversible state of “moderate” brain damage and dementia), that aspect of the FDA approval also merits careful attention.

Treatment of Moderate-Stage Alzheimer's Disease with Memantine

The FDA's approval (in 2004) of memantine, for treating patients who have reached a state of “moderate” Alzheimer's disease, arose primarily due to the actions and efforts of two different and distinct groups of researchers, who were working effectively in parallel with each other. One group included employees of Merz Pharmaceuticals, the German company which developed memantine, and which markets it. The second group was led by Dr. Stuart Lipton, a researcher at the Sanford-Burnham Medical Research Institute, in California.

Dr. Lipton's role requires attention, because he has claimed, in numerous publications, to be the one who discovered what he asserts to be the neuronal mechanism by which memantine can prevent various types of neurodegeneration, in various neurological diseases and disorders. An example of that claim, made in a press release issued by his institute (which would not have been released, without Lipton's knowledge and approval) was published at www.scienceblog.com/community/older/2004/2/20041218.shtml. It states:
“Dr. Lipton was the first to discover a unique mechanism of action for the drug Memantine, allowing it to work only on pathological conditions and not affect ‘normal’ behavior. This discovery led to clinical development of Memantine and its subsequent approval by the European Union and the United States' Food and Drug Administration for treatment of Alzheimer's Disease as the first neuroprotective drug.”
   More detailed versions of that claim by Dr. Lipton are included in numerous papers he coauthored, including Lipton et al 2004 (published the same year that FDA approval was granted), which was entitled, “Paradigm shift in NMDA receptor antagonist drug development: molecular mechanism of uncompetitive inhibition by memantine in the treatment of Alzheimer's disease and other neurologic disorders.”

That article (and numerous similar articles by Lipton and his coworkers, including Xia et al 2010) advocated and promoted Lipton's theory that: (i) NMDA receptors are overly active (hyperactive) in Alzheimer's disease; and (ii) the chronic excessive stimulation of neurons, due to NMDA receptor hyperactivity, is a factor which likely aggravates and accelerates the neurodegenerative process, in Alzheimer patients.

Accordingly, Lipton and his coworkers asserted that memantine can bind to a site in the NMDA receptor ion channel in an unusually rapid “on/off” fashion, which (in their belief) allows memantine to have significant effects in only those neurons that are being subjected to hyperactivity, without having substantial effects on other neurons that are not being subjected to NMDA receptor hyperactivity. In other words, according to those articles, because of the rapid “on/off” kinetics of memantine, it can protect those neurons which are most at risk, in Alzheimer patients, without interfering with other neurons that are not facing being stressed and damaged by NMDA hyperactivity and excitotoxicity.

That theory, hypothesis, and belief was set forth in language such as the following, from Lipton et al 2004:

“Here we review the molecular mechanism of memantine's action and also the basis for the drug's use in these neurological diseases, which are mediated at least in part by excitotoxicity. Excitotoxicity is defined as excessive exposure to the neurotransmitter glutamate or overstimulation of its membrane receptors, leading to neuronal injury or death. Excitotoxic neuronal cell death is mediated in part by overactivation of NMDA-type glutamate receptors, which results in excessive Ca(2+) influx through the receptor's associated ion channel. Physiological NMDA receptor activity, however, is also essential for normal neuronal function. This means that potential neuroprotective agents that block virtually all NMDA receptor activity will very likely have unacceptable clinical side effects. For this reason, many previous NMDA receptor antagonists have disappointingly failed advanced clinical trials for a number of neurodegenerative disorders. In contrast, studies in our laboratory have shown that the adamantane derivative, memantine, preferentially blocks excessive NMDA receptor activity without disrupting normal activity. Memantine does this through its action as an uncompetitive, low-affinity, open-channel blocker; it enters the receptor-associated ion channel preferentially when it is excessively open, and, most importantly, its off-rate is relatively fast so that it does not substantially accumulate in the channel to interfere with normal synaptic transmission.”

It is not clear whether (or to what extent) a review panel at the FDA takes those types of assertions and hypotheses (i.e., concerning purported cellular mechanisms of action) into account, when deciding whether to approve an application for public sale of a new drug. The FDA typically asserts that its decisions are based solely on evidence of whether a drug actually improved or alleviated a measurable problem or symptom caused by some disease, as shown by statistical data from a large-scale clinical trial that must include thousands of volunteers, at multiple study sites. Nevertheless, the FDA's review panels are composed of experts in the field, and those experts will inevitably want to consider anything that an advocate for a new drug application has said or written, about possible cellular or biochemical mechanisms which might help explain why a new drug candidate is believed to offer one or more benefits, to treated patients.

Accordingly, the FDA approval for the use of memantine, in Alzheimer patients who had reached a level of “moderate” dementia, was based on data from clinical trials which indicated that middle-stage Alzheimer patients who were treated with a low dose of memantine showed a transient and slight improvements in (i) a “global” assessment of cognition; and, (ii) the patients' ability to manage various activities in their daily lives. That benefit was not found or demonstrated among patients in the early stages of Alzheimer's disease; and, the data also indicated that memantine: (i) did not prevent further deterioration of cognitive function; and, (ii) could not significantly slow down the rate or progression of overall neurodegeneration, in Alzheimer patients.

In the opinions of the Applicants herein, there are serious and substantial reasons for questioning, challenging, and doubting the theories, hypotheses, and arguments that were set forth by Lipton's research group, and by various employees of Merz, during their efforts to obtain FDA approval for public sale of memantine.

A powerful reason for questioning and challenging Lipton's theories and assertions, concerning a possible beneficial mechanism for memantine in Alzheimer patients, arises from numerous published medical and scientific reports which state and demonstrate, apparently with a high level of consistency, that excessive NMDA receptor activity is NOT a condition which is occurring, either in the brains of aging people in general, or in the brains of Alzheimer patients in particular. Indeed, those reports consistently report and describe the exact opposite condition, i.e., that decreased activity and capacity, in the NMDA receptor system, is one of the conditions and apparent problems that gradually arise and grow worse, as humans or test animals pass beyond middle age, and into the stages that can be referred to as elderly, senescent, etc. Indeed, that gradual loss and decrease, in the numbers and activity levels of NMDA receptors, is even more prevalent, and apparently is an aggravating factor, in patients who are suffering from Alzheimer's disease.

For example, a number of published studies (Tamaru et al 1991, Gonzales et al 1991, Liang et al 1992, and Magnusson et al 1993 offer examples) contain data from rodent tests, which consistently indicated that with advancing age, there is a decrease in the functional activity and capacity of the NMDA transmitter system. Wenk et al 1991 reported that similar losses in NMDA receptor activity were seen in elderly monkeys, and numerous articles (including Piggott et al 1992, Ulas et al 1997, Sze 2001, and Bi et al 2002) have reported similar findings in humans.

Those analyses and conclusions are based on objective, impartial, and quantitative measurements of certain types of biochemical indicators, mostly involving three different types of tests on tissue samples from brains. Those three types of tests involve: (1) analyzing quantities of messenger RNA (mRNA) strands which encode the specific proteins that are assembled into NMDA receptor complexes; (2) analyzing the binding concentrations or densities of certain types of “ligand” molecules which bind only to certain known receptor types, and which have been “labeled” with radioactive isotopes (such as 3H) that enable various types of measurements and imaging; and, (3) measuring the extent to which certain probe drugs (either agonists or antagonists) can either provoke or suppress various types of known and measurable neuronal responses to various stimuli or conditions.

When those types of biochemical indicators, in brain tissues from elderly animals or humans, are compared to identically-treated brain samples from young or middle-aged adults, the decreases in the indicators of NMDA receptor capacity or activity are pronounced, and pass beyond any level of statistical uncertainty. In elderly but non-Alzheimer animals and humans, the decreases in NMDA receptor numbers and/or activity levels reportedly range from about 15% to about 40% (e.g., Gonzales et al 1991, Magnusson et al 1995).

In addition to the NMDA receptor decreases that occur during normal aging, there are also major additional and further losses in NMDA receptor numbers and activity levels in the brains of Alzheimer patients, compared to tissue samples from non-Alzheimer adults who were matched for age, gender, and other factors. A number of reports indicate that those Alzheimer-related losses commonly are in the range of about 35 to 55% (e.g., Janson et al 1990, Ulas et al 1997, Bi et al 2002, etc.), compared to age-matched non-Alzheimer samples.

In addition, still other published reports indicate that beta-amyloid molecules, when present in the form which generates the beta-amyloid plaques that are a primary and defining characteristic of Alzheimer disease, inflict major and severe disruptions on the NMDA receptor system (e.g., Snyder et al 2005; Yamin et al 2009). One can reasonably presume that those types of beta-amyloid disruptions to the NMDA system will be factored into the results of any ligand-binding tests, but will not be factored into tests which measure mRNA levels.

When those factors are combined and taken jointly into account, it becomes clear that Alzheimer patients suffer from deep and profound losses in NMDA receptor numbers and activity. Based on simple numbers, if normal elderly people have only about 70% of the NMDA receptor capacity of young adults (reflecting a 30% average loss, based on aging alone), then an Alzheimer patient who has lost an additional 40% of his NMDA receptor capacity, compared to a healthy person of the same age, will have only about 42% (0.6×0.7=0.42) of the NMDA receptor capacity he had, as a young adult.

Accordingly, one must seriously question whether it makes any logical sense, at all, to treat Alzheimer patients—who are already suffering from deep and profound impairments and losses in their NMDA receptor systems, which play crucial and absolutely essential roles in mental processes such as memory and learning—with an NMDA antagonist drug, which by its very nature and definition will inflict and impose even greater reduction and impairments on top of the already-reduced activity levels in those patients' NMDA receptor systems.

Anyone who is aware of the actual facts, and who knows about the severe declines and reductions in the numbers and activity levels of NMDA receptors among the elderly in general, and among Alzheimer patients in particular, should seriously question, without hostility but with an informed level of skepticism, any published theories which state that a drug which further suppresses and slows down NMDA receptor activity, will somehow make things better, in the brains of patients who are suffering from NMDA receptor activity levels that are already impaired, inadequate, and too low (rather than being dangerously high, as implied by Lipton's articles).

Furthermore, if Lipton's purported mechanism of action is indeed an actual protective mechanism which enables memantine to protect and benefit the brains of people suffering from Alzheimer's disease, then two important questions arise. The first question is this: if Lipton's theory is correct, then why did memantine fail to provide any significant benefits or advantages, in the brains of Alzheimer patients who were still in the early stages of the disease, and who had not yet reached a point where their brains had already been irreparably damaged? During the early stages of the disease, when the damage is not yet so widespread, any memantine presumably would have a greater number of target sites where it would be able to offer benefits, if indeed it is offering the benefits which Lipton claims. The very nature and basis of Lipton's claim is that memantine can help protect a subset of neurons—i.e., those neurons which are being stressed and potentially damaged, by too much NMDA receptor activity—from being killed, by those stresses. If that is indeed how memantine works, then one would logically expect it to work best during the early stages of the disease. However, that was not found to be the case, during the clinical trials.

The second question is this: if Lipton's theory is correct, then why did memantine fail to slow down the progression of neurodegeneration, in Alzheimer patients who were treated with memantine?

The central basis and foundation of Lipton's claim (i.e., that he discovered a “paradigm shift” in treatments for patients with Alzheimer's disease) is his assertion that memantine functions in what can be called a “sweet spot”, which arose from its rapid on/off pharmacokinetic behavior. According to Lipton's theory: (i) memantine will significantly affect (and thereby benefit, and protect) those neurons which are under excitotoxic stress and risk because of purported NMDA receptor hyperactivity; while, (ii) memantine will not significantly affect neurons which are behaving normally, and which are not being subjected to excitotoxic stress or risk due to NMDA receptor hyperactivity. However, if that claim by Lipton is true and valid, then that is exactly the type of mechanism, activity, and effect which presumably should be able to help and benefit early-stage Alzheimer patients, at least as much as (and presumably even more than) Alzheimer patients who have already reached a middle stage of brain damage. In addition, if Lipton's asserted mechanism of action is indeed occurring, then memantine should be able to at least help slow down the continuing onslaught of further neurodegeneration, in Alzheimer's disease. However, the clinical trial data clearly showed that memantine could not provide or accomplish either of those two benefits.

Accordingly, any objective observer is entitled (and arguably even obliged) to ask whether the theoretical mechanism of action that was asserted and claimed by Lipton is consistent with, or supported by, the actual results and data from the clinical trials.

The Applicants herein do not wish to take an overtly aggressive or antagonistic approach toward anyone who is legitimately and seriously trying to analyze and learn more about brain functioning, neuropharmacology, and improved ways to treat Alzheimer's disease, or any of the other devastating neurological disorders that have effectively thwarted, frustrated, and defied all efforts to date to find cures. However, based on everything they have learned (over multiple decades of serious and focused research) about NMDA receptors, NMDA antagonist drugs, and “safener” drugs which can block the neurotoxic side effects of NMDA antagonist drugs, they are convinced that the use of even a mild NMDA antagonist drug—if administered to patients who are already suffering from abnormally low, insufficient, and inadequate levels of NMDA receptor activity—is not a proper treatment, and poses serious threats of doing more damage than good, in such patients.

Those risks of lasting damage, in actual Alzheimer patients, are aggravated by the fact that there is no clear boundary line, or diagnostic distinction, between the “early” and “moderate” stages of Alzheimer's disease. Therefore, as a result, any treating physician who is trying to determine whether an Alzheimer patient has reached a “moderate” level of brain damage (so that memantine treatment can commence, as allowed by the terms and limits of the FDA's approval), will and must rely, to at least some extent, upon comments by caregivers and family members, concerning various actions, behaviors, and attempts to communicate by an Alzheimer patient. For obvious reasons, any relative or other caregiver who is struggling to cope with someone who clearly has Alzheimer's disease, even if it is only in an “early” stage, will inevitably want to try any treatment that holds any promise (or even hope) for any potential benefits. Accordingly, physicians who must deal with such relatives and caregivers are inevitably asked and often pressured to go ahead and begin treating such patients with memantine, as soon as possible, just in case it may be able to help.

In view of those factors, the Applicants herein are deeply concerned that, rather than helping such patients, memantine is very likely to be causing more damage than good, to the extent that it does indeed suppress and reduce NMDA receptor activity in Alzheimer patients, at the dosages that are being used.

However, it may well be that such concerns, by the Applicants, are not especially serious or acute, in view of the relatively low dosages of memantine that are actually being administered to Alzheimer patients. The Applicants have tested memantine, in animals, to measure and quantify its ability to protect against excitotoxic neurodegeneration, using several standard animal models that are used to test and measure excitotoxic neurodegeneration. The results of those tests indicate that memantine can indeed offer some level of protection against excitotoxic damage, in those animal models, but only when administered at very high doses, at levels which cause severe functional and behavioral disturbances (Creeley et al 2006 and 2008). Therefore, it is very unlikely that memantine is being administered, to Alzheimer patients, at dosages that are actually high enough to prevent excitotoxic neurodegeneration, since the dosages being used in human patients are not causing the types of observable functional disturbances that have been seen when anti-excitotoxic dosages are administered.

Accordingly, the Applicants herein suspect that any beneficial effects that occur, in memantine-treated moderate-stage Alzheimer patients, may be arising from either or both of two completely different factors. One factor involves activity at an entirely different receptor type. In specific, memantine was found to suppress activity at the nicotinic subclass of acetylcholine (ACh) receptors (e.g., Buisson et al 1998, Oliver et al 2001, Maskell et al 2003, Aracava et al 2005), but it also was discovered to stimulate activity at the muscarinic subclass of ACh receptors, in ways which improved performance on behavioral tests (Dreyer et al 2007). If that reported activity and effect of memantine is occurring at significant levels, inside the brains of people with mid-stage Alzheimer's disease, then it would align with the effects of donepezil (sold under the trademark ARICEPT), but with an important difference which may lead to significant benefits, and which may help explain why a number of reports have indicated that a combination of both donepezil (ARICEPT) and memantine (NAMENDA) appears to offer greater benefits, to many patients, than either drug alone.

As mentioned above, donepezil inhibits cholinesterase, the enzyme which normally breaks apart and deactivates extracellular ACh, after the ACh has triggered an ACh receptor. As a result, donepezil provides extracellular ACh inside the brain with a longer “lifespan”, which in turn leads to greater ACh activity levels. Since extracellular ACh cannot distinguish between muscarinic versus nicotinic ACh receptors, it activates both subclasses, equally. However, various factors tend to suggest that muscarinic receptors have greater levels of utility and benefit, inside the brain, than nicotinic receptors. Accordingly, if memantine can mildly increase activity at muscarinic ACh receptors, while also mildly inhibiting activity at nicotinic ACh receptors (in a way which is likely to bring those nicotinic receptor activities back down closer to normal levels despite the artificial boost caused by the donepezil), then memantine (and memantine-plus-donepezil combinations) may be benefiting Alzheimer patients through an entirely different mechanism than the one which was postulated by Lipton's research group and various other researchers at Merz Pharmaceuticals.

The second potential mechanism of action, for memantine, which merits at least some consideration, arises from its potential to act as a tranquilizer and/or anxiolytic drug, and possibly even as a mild sedative. Since it mildly blocks not just one but two major subclasses of excitatory receptors (i.e., the NMDA class of glutamate receptors, and the nicotinic class of ACh receptors), and since it is, in effect, a much milder version of ketamine, which can be either a sedative or an anesthetic (depending on the dosage), the likelihood that memantine can and will exert some level of tranquilizing, anxiolytic, and/or sedating effects cannot and should not be ignored. As physicians and caregivers are well aware, problems such as agitation, delusions and even hallucinations, and impaired sleep tend to plague and distract Alzheimer patients, and it is standard practice for physicians to prescribe an assortment of sedatives, tranquilizers, anxiolytics, and sleep-inducing drugs. Because of how it works, memantine would appear to be capable of exerting at least some level of each and all of those four effects, and it apparently can do so via a mechanism (i.e., NMDA receptor suppression) which is independent from and generally unrelated to the cellular and neuronal mechanisms that are used by all other classes of tranquilizers, anxiolytics, sedatives, and sleep-inducing drugs. Accordingly, since it appears to be a relatively mild and well-tolerated drug, if it can provide any or all of those benefits, in Alzheimer patients, then it can provide an important and potentially very useful component, in the array of potentially useful drugs that physicians can use, in their efforts to alleviate the symptoms of Alzheimer's disease, among patients who have already suffered serious and irreversible brain damage.

The above information, pertaining to memantine and Alzheimer's disease, offers a reasonable and realistic view of how advancement in medical science tends to occur in fits and starts. For advancement to occur, scientists must propose hypotheses, and then test and publish their hypotheses. Others must then join in a larger form of ongoing testing and validation, to reject or at least clarify and determine the limits of those that are incorrect or too broad, and to build upon those that are confirmed, and that can shed new light on biological processes.

For better or worse, the Lipton/Merz claims about how memantine might work, in the brains of Alzheimer patients, were widely and forcefully disseminated, in bold and confident language, into a medical establishment that was eager and even desperate to announce any type of progress, advance, or breakthrough against a lingering, terrible, and eventually fatal disease that has no cure. Even though the grand and exciting theories did not line up well, at all, with the miniscule actual benefits that were seen in clinical trials, the grand and exciting theories began to be repeated uncritically, not so much by neurology researchers as by treating physicians, who wanted to be able to offer their patients not just hope, but additional treatment options (which directly translates into increased income for treating physicians) which could be tried, tested, and evaluated on essentially all Alzheimer patients (since those patients have no other options that are any better).

Accordingly, to bring this brief bit of analysis to a close, a certain point merits careful attention and emphasis, at this time. The fact is that the “paradigm shift” and theories that were proclaimed by the Lipton group and by Merz researchers, which apparently were accepted by the world-class experts who served on the FDA review panel which approved memantine, unavoidably conflict with, and teach away from, the mechanisms and theories set forth by the Applicants herein. The heart and essence of the invention described herein can be summarized as follows:

(i) a treatment which uses an NMDA antagonist drug, to solve or at least alleviate a severe and major neurological disorder which requires medical intervention, cannot be optimally effective, unless the NMDA antagonist drug is administered in a dosage/duration regimen which will cause serious neurotoxic side effects (or at least severe and unacceptable risks of neurotoxicity), if administered in the absence of at least one “safener” drug; and,

(ii) in order to provide the type of optimal and highly potent safening effects which will be necessary, in order to provide adequate protection during the types of life-changing, brain-rewiring interventions described herein, at least two different safening drugs, which work by substantially different mechanisms as described herein, must be coadministered, along with the NMDA antagonist drug.

There simply is no way to reconcile that teaching and invention, with the claims of Lipton and others who have argued and claimed that memantine represents a safe and effective “paradigm shift” which arose from a truly effective and yet safe NMDA antagonist drug that can be administered for long periods of time. Accordingly, the current invention clearly is not “obvious to anyone with ordinary skill in the art,” since it directly contradicts a major hypothesis which has been widely proclaimed, and widely accepted by those skilled in the art.

This leads back to the larger point being made by this section of this application. In this field of neuropharmacology in particular, anyone who is attempting to evaluate the so-called “prior art” must have a reasonable and informed awareness of where any particular publication or assertion stands, and how it would be regarded by genuine experts, on a spectrum or continuum that ranges from “accepted truth”, at one end, to “unsupported theory” at the other end. The best and most reliable way to evaluate the state of the art and what is “known”, in this particular branch of neuropharmacology, is by asking, “What is actually being done, today, by physicians and drug companies, to treat these severe and debilitating neurologic problems? What are the best and most widely respected and used treatments and drug regimens, for treating major neurologic problems that are widespread and well-known?” Clearly, under the current state of the art, none of the drug combinations described herein are being used, by physicians, to treat any types of major neurologic problems.

Therefore, one object of this invention is to disclose safener drug combinations that are sufficiently potent and effective to reliably prevent the neurotoxic side effects and permanent brain damage that would otherwise be caused, if an NMDA antagonist drug is being administered at a dosage sufficient to prevent and minimize the excitotoxic brain damage that is occurring in a patient suffering from a major acute crisis, such as a stroke, cardiac arrest, severe head injury, or other hypoxic or ischemic assault on the brain.

Another object of this invention is to disclose safener drug combinations that are sufficiently potent and effective to reliably prevent the neurotoxic side effects and permanent brain damage that would otherwise be caused by a potent NMDA antagonist drug, even when the NMDA antagonist drug is being administered at a heavy and prolonged dosage in order to establish a lasting or permanent alteration in the central nervous system of a patient suffering from a severe neurologic disorder involving NMDA receptor hypersensitivity or hyperactivity.

A third object of this invention is to establish that there are direct correlations between: (i) the efficacy and potency of various candidate safener drug combinations, in preventing hallucinations and other psychotomimetic effects in humans, and (ii) the efficacy and potency of those same candidate safener drug combinations, in preventing neurotoxic injury to CNS neurons, and in minimizing or preventing permanent brain damage, which would otherwise be cause by administration of potent NMDA antagonists using heavy and/or sustained dosage regimens.

These and other objects of the invention will become more apparent through the following summary and detailed description of the invention.

### SUMMARY OF THE INVENTION

This invention discloses combinations and mixtures of certain types of “safener” drugs which, when combined, can reduce or prevent the neurotoxic and psychotomimetic side effects of NMDA antagonist drugs such as ketamine. These safener drugs will be used as secondary drugs (which can also be called adjunctive drugs, or similar terms), to block and prevent the potentially toxic side effects of a “primary” drug (i.e., an NMDA antagonist drug, such as ketamine) that is being administered to a patient who is suffering from a severe neurological problem or disorder.

In order to establish an enduring and truly effective alteration in a patient's nervous system, in a manner which can provide lasting and even permanent relief from a severe and chronic disorder, the NMDA antagonist (such as ketamine) preferably should be administered to the patient at a relatively high dosage, and for a prolonged period of time, such as continuously, 24 hours per day, over a span of multiple days in succession. However, because of the crucial roles that NMDA receptors perform throughout the central nervous system of any mammal, it is believed and asserted by the Applicants herein that any prolonged drug-induced suppression of NMDA receptor activity will pose a severe and unacceptable risk of permanent neurotoxic brain damage, unless a highly potent combination of at least two distinct “safener drugs”, which act via different neuronal receptor systems, is coadministered to the patient along with the NMDA antagonist drug.

In particular, the Applicants herein have discovered and disclosed that in order to prevent (with an adequate margin of safety) the type of brain damage which can be proven (in animal tests) to arise from prolonged drug-induced suppression of NMDA receptors, activity at two other classes of receptors must also be suppressed, in a substantial and yet controlled manner. Those two classes of receptors are: (1) the muscarinic class of acetylcholine (ACh) receptors, including (but not necessarily limited to) the M3 subclass of muscarinic receptors; and, (2) at least one and preferably both of the two non-NMDA types of glutamate receptors (i.e., kainate receptors and AMPA receptors). Several drugs that are known to suppress activity at either one (but not both) of those two additional classes of receptors are disclosed herein. They can be mixed with each other, to form safener drug mixtures that will simultaneously suppress activity at muscarinic ACh receptors, and at non-NMDA glutamate receptors. Accordingly, these mixtures, which (to the best of the Applicants' knowledge) have never previously been created, will provide highly effective, potent, and reliable safening activity against the risk that permanent brain damage would otherwise be created by a treatment regimen that uses an NMDA antagonist drug, such as ketamine, at a dosage large enough to provoke lasting and even permanent changes in the patient's central nervous system.

It is also disclosed herein that parallel types of testing, involving laboratory animals in some tests and healthy human volunteers in other tests, has revealed that there are direct and apparently reliable correlations between: (i) the ability of various candidate safener drugs to block and suppress hallucinations and other psychotomimetic displays, symptoms, and manifestations caused by ketamine, in humans; and, (ii) the ability of those same candidate safener drugs, at corresponding dosages, to block and suppress the symptoms and displays of neuronal stress and damage that are caused by ketamine in animal brains. Accordingly, the discovery and recognition of that useful and apparently reliable correlation provides powerful tools and methods which enable those skilled in the art to determine, using no more than routine experimentation:

(i) recommended dosage ranges, for treating different patients suffering from any of several different types of neurologic disorders that require pharmaceutical intervention; and,

(ii) optimal relative dosages, for any specific combination of two or more selected safener drugs, for patients undergoing sustained therapy using an NMDA antagonist (such as ketamine) for any of various different types of neurologic disorders which can be treated by these drug regimens.

### BRIEF DESCRIPTION OF THE DRAWINGS

FIG. 1 is a schematic depiction of several neurotransmitter pathways in a healthy brain. In this system, a glutamate control neuron 10 releases glutamate, which stimulates NMDA receptors on several inhibitor neurons 20-50. Stimulation by glutamate causes the inhibitor neurons to release inhibitory neurotransmitters, such as GABA or norepinephrine. The inhibitory neurotransmitters help suppress unwanted excitation of exciter neurons 60-80. This glutamate-driven system, which establishes control over exciter neurons 60-80, helps protect a target neuron, shown as pyramidal neuron 100 in the posterior cingulate or retrosplenial (PC/RS) cortex. If a potent NMDA antagonist is administered, the glutamate-mediated control of inhibitor neurons 20-50 and exciter neurons 60-80 is impaired, and exciter neurons 60-80 will begin to bombard pyramidal neuron 100 with abnormally large numbers of nerve impulses, causing hallucinations and other psychotomimetic symptoms and stresses, at levels which can kill pyramidal neurons in vulnerable regions of the brain. However, if a mixture of two or more selected safener drugs is used to simultaneously block two or more types of excitatory receptors on the pyramidal neurons, those neurons will be protected against the overload of nerve signals, and will not suffer permanent damage, even if the NMDA antagonist treatment is sustained for days, continuously, in order to “reset” hyper-sensitized NMDA receptors.

FIG. 2 is an alternate illustration of the same types of neurotransmitter pathways that are depicted in FIG. 1, arranged in a flow chart arrangement, to indicate the sequential nature and stages of the types of neurons that become involved when a prolonged treatment is carried out using an NMDA antagonist such as ketamine.

### DETAILED DESCRIPTION

As briefly summarized above, this invention discloses combinations and mixtures of certain types of drugs, referred to herein as “safener drugs” since they will be used to reduce and prevent certain unwanted and apparently toxic side effects that otherwise would be caused by one or more other drugs.

In this invention, the “primary” drug is an NMDA antagonist, such as ketamine. When used for only brief periods of time (such as for anesthesia during surgery), and when used in conjunction with an anxiolytic or sedative drug (which is conventional practice, to minimize the side effects known as “ketamine emergence reactions”), ketamine is relatively safe. However, ketamine and other NMDA antagonist drugs offer potentially huge medical and therapeutic benefits, if they can be administered in substantially higher dosages, up to and including dosage and duration combinations that can allow a human central nervous system to effectively “reset” NMDA receptors that have become hyper-sensitized or hyper-active in ways that are causing pathological problems.

If that type of NMDA antagonist therapy, using a relatively heavy dosage-and-duration regimen to “rewire” and reset certain neuronal circuits and connections in ways that will provide lasting and hopefully permanent changes in a central nervous system, is being used to treat a serious pathological disorder, then it will create unacceptable risks of unwanted neurotoxic side effects and potentially permanent brain damage, unless strong and effective steps are taken to prevent those types of unwanted side effects. Accordingly, this invention discloses mixtures and combinations of “safener drugs” which can act together in a synergistic manner, to reduce and prevent the unwanted side effects, to a point where an otherwise unacceptable risk of permanent brain damage can be prevented, or at least reduced to an absolute minimum, to a point which will render prolonged treatments, with relatively large dosages of dangerous drugs, to become safe and tolerable.

One of the teachings of this invention is that it is useless, and a waste of time, effort, and resources, to try to determine and then utilize a dosage of a NMDA antagonist drug, for a continuous subanesthetic treatment regimen, which is “barely enough and not too much, because it's dangerous”. If the NMDA antagonist will be administered in a dosage regimen that is intended to permanently alter a damaged or dysfunctional nervous system, in a patient who requires major medical intervention to try to resolve a severe problem, then the basic purpose and essence of the treatment would be thwarted, or at least severely jeopardized and almost certainly rendered less effective, less reliable, and less enduring, if a “barely enough” approach is used, or even attempted. Instead of attempting to develop and use that type of approach, any physician who wishes to use a sustained and prolonged treatment as disclosed herein should realize and accept that the only way an NMDA antagonist drug will be able to efficiently create and then reliably “nail down” a lasting improvement, which can continue to provide benefits for years after the treatment has been completed and terminated, will require administration of a strong drug, at a potent and change-inducing dosage, for a continuous and prolonged period of time, such as for multiple days in a row, without interruption.

Therefore, rather than trying to sidestep, ignore, or attempt to “tweak” and tinker with the risk that dangerous and toxic side effects will be caused by such a treatment, it would appear that the most effective and reliable approach to this type of brain-altering and life-altering medical treatment will require physicians to: (i) face up squarely to the concern that large risks will indeed be created, as unwanted and apparently neurotoxic side effects, if an NMDA antagonist drug is administered at a dosage-duration regimen sufficient to permanently alter and “rewire” a patient's brain; and, (ii) use a mixture of potent and selected safener drugs, in combination, to prevent any such toxic and damaging side effects, and to bring otherwise unacceptably high levels of risk down to a minimal and tolerable levels of very low risk.

That is what this invention does. Through a combination of animal tests and human trials, it has been discovered and recognized by the Applicants herein that excitatory activity at two specific classes of synaptic receptors, which are present in high numbers on the surfaces of certain types of brain cells in regions of the brain that are highly susceptible to neurotoxic damage from a sustained NMDA antagonist treatment, must be simultaneously suppressed.

The two different types of neuronal receptors which play major roles in the safening activity discussed herein, and which must be simultaneously suppressed in order to achieve safening and protection levels which are sufficiently reliable to meet the requirement for using “best known practices” when treating patients who are undergoing life-altering therapy, are:

(1) the muscarinic class of acetylcholine (ACh) receptors; and,

(2) at least one and preferably both of the two non-NMDA classes of glutamate receptors (i.e., kainic acid (KA) and AMPA receptors).

Rather than attempting to identify and use a single type of safener drug which has substantial levels of activity-suppressing effects at both of those two classes of receptors, a more effective and reliable (and therefore preferable, at least through early clinical trials) approach is to use a mixture of at least two different drugs, where each component of the mixture has been selected because it is both (i) sufficiently potent, and (ii) sufficiently selective, to perform its necessary task in an effective and reliable manner, with a margin of safety that is adequate to avoid and prevent what otherwise might become an entire remaining lifetime of serious brain damage and mental impairment. Since that is exactly what is at stake, when powerful drugs are being used to permanently alter a seriously damaged and/or dysfunctional central nervous system, an essential part of this invention arises from and resides within the twin realizations that:

(i) potent and reliable levels of safening activity must be provided, for any patient who is undergoing the type of brain-altering treatment described herein, in order to bring unacceptably high levels of risk down to acceptably low and tolerable levels; and,

(ii) the best and most reliable way (by far) to provide the appropriate levels of protection and safety, in the types of treatments discussed herein, is by selecting and using a combination of at least two different safener drugs, in a manner and at a suitable dosage which allows each component of the mixture to perform a known, specific, and selective role, in an effective and reliable manner.

The appropriate levels of safety and reliability cannot be achieved by compromising, such as by trying to identify and then use a drug which can suppress muscarinic ACh receptors to some extent and also suppress non-NMDA glutamate receptors to some extent, but which cannot do either task in a fully and predictably reliable manner because a dosage must be divided and allocated between those very different classes of neuronal receptor types. This factor becomes even more of a concern in view of the variations that exist between protein sequences, protein expression levels, and other cellular, biochemical, and physiological factors among individuals with different ancestries, genotypes, and neurologic problems.

Accordingly, now that a combination of specific receptor types have been identified which must have their activities simultaneously suppressed, during and throughout the entire course of a prolonged treatment using an NMDA antagonist drug such as ketamine, the selection of two or more drugs which can accomplish that type of simultaneous suppression of two or more different receptor types is fully within the skill of the art, using drugs that are already known.

It is important to understand that two main factors come into play, in analyzing these types of drugs.

The first main factor depends upon which neuronal receptor types are affected. This factor clearly distinguishes between: (1) agents that reduce activity at the muscarinic class of acetylcholine receptors; and (2) agents that reduce activity at non-NMDA glutamate receptors.

If desired, safener drugs which interact with non-NMDA receptors can be further subdivided, to include, within a complete safening mixture:

(2a) a first drug which selectively reduces activity at the KA class of non-NMDA glutamate receptors without affecting the AMPA class of receptors; and,

(2b) a second drug which selectively reduces activity at the AMPA class of non-NMDA glutamate receptors without affecting the KA class of non-NMDA glutamate receptors.

Because of the structural similarities and “cross-affinities” that exist between the KA and AMPA subclasses of non-NMDA receptors, and because certain types of drugs (mainly analogs of willardiine, as described in a number of published articles cited in the Background section) are known to suppress activity at both of those two classes of non-NMDA receptors, the question of whether an optimal safener mixture should contain three different agents, all of which utilize competitive binding reactions (i.e., an anticholinergic agent which binds to and occupies muscarinic ACh receptors, a selective KA receptor antagonist which binds to and occupies KA receptors, and a selective AMPA receptor antagonist which binds to and occupies AMPA receptors), or only two such agents (i.e., an anticholinergic agent, and a non-NMDA receptor antagonist which binds to and occupies both KA and AMPA receptors), can be evaluated, using the methods and compounds disclosed herein.

The differences between those two marginally different approaches are not critical to this invention, and belong in the realm of optimizing and “tweaking” the invention. What is critical to this invention is the simultaneous suppression of activity at both (i) the muscarinic class of ACh receptors, and (ii) non-NMDA receptors (which presumably will require at least some level of suppression of both the KA subclass of non-NMDA receptors, and the AMPA subclass of non-NMDA receptors.) This issue and factor will also require attention to the second main factor, discussed below.

The second main factor which requires attention, in the selection of optimal components for a safener drug mixture, arises from the fact that either of two different modes of action can be used, to reduce activity at a specific targeted class of neuronal receptors. Those two different modes of action involve:

(i) competitive binding reactions, in which drug molecules bind to and occupy certain types of receptor proteins on the synaptic surfaces of neurons, in a manner which prevents the natural neurotransmitter molecules (either ACh, or glutamate) from contacting and activating those receptor proteins; or,

(ii) neuronal reactions which suppress the release, by neurons in the brain, of ACh and/or glutamate (i.e., the two main types of excitatory neurotransmitters). If a drug suppresses the neuronal release of acetylcholine, inside the brain, then as a direct result, that drug will suppress activity levels at muscarinic ACh receptors inside the brain. Similarly, if a drug suppresses the neuronal release of glutamate, inside the brain, then that drug will suppress activity levels at both KA and AMPA receptors.

Both of these two modes of action (i.e., competitive binding to receptors, and suppression of excitatory transmitter release) are well-recognized and understood by neurologists, and an assortment of drugs are known and available which can exploit and utilize either of those two approaches.

Accordingly, candidate safener drugs that are of interest in this invention can be grouped into four major categories, as shown in Table 1, in which: (i) the two vertical columns indicate which receptor types are having their activity levels suppressed; and (ii) the two horizontal rows indicate whether the suppressing effects arise from (i) competitive binding to glutamate or ACh receptors, or (ii) inhibition of glutamate or ACh release.

TABLE 1

| Mode of | Non-NMDA (KA/AMPA) | Muscarinic ACh |
|---|---|---|
| action | receptor suppression | receptor suppression |
| Competitive | CATEGORY 1A IN TEXT | CATEGORY 2A IN TEXT: |
| Binding | Willardiine analogs (e.g., | scopolamine, atropine, |
| To | ACET, ATPA, | benztropine, |
| Receptors | 5-iodowillardiine, LU 73068, | amitriptyline, olanzapine, |
| | 97175 and 112313, | etc. ACAA compounds |
| | LY382884, NS1209, | (procyclidine, biperiden, |
| | UBP277 and 282, etc). | trihexyphenidyl, etc). |
| | talampanel, topiramate, | TCAA compounds |
| | GYKI-52466, | (ethopropazine, etc). |
| | LU 115455 and 36541, | |
| | thieno-oxazine | |
| Inhibition | CATEGORY 1B IN TEXT | CATEGORY 2B IN TEXT |
| of | Sodium channel blockers | Alpha-2 adrenergic agonists |
| Neuro- | (riluzole, lamotrigine, | (clonidine, iodoclonidine, |
| transmitter | carbamazepine, | guanabenz, etc.) |
| Release | diphenylhydantoin, etc.) | GABA-A agonists |
| | Calcium channel blockers | barbiturates, |
| | (gabapentin, pregabalin, etc.) | benzodiazepines, etc. |

Safener Category 1A: Agents that Suppress Activity at Non-NMDA Receptors by Competitive Binding

As mentioned in the Background section, a number of drugs suppress activity at non-NMDA glutamate (NNG) receptors, by binding to KA and/or AMPA receptors, in a manner which prevents glutamate from reaching and contacting its normal binding sites. As described above, most of these types of candidate drugs were identified after a compound called willardiine was discovered to activate non-NMDA receptors. After that discovery, researchers began searching for chemical modifications to willardiine, and identified a number of analogs and variants which will bind to AMPA and/or kainate receptors, and: (i) which do not activate those receptors; or, (ii) which activate those receptors only once, and then remain bound to the AMPA and/or kainate receptors for an abnormally long period of time, in ways that prevent and suppress subsequent firing events that otherwise would occur.

It should also be noted that “allosteric” binding drugs are classified and regarded as “competitive” binding agents, as used herein, so long as the allosteric agent binds to a glutamate receptor complex in a manner which suppresses activity at that receptor, regardless of whether the allosteric drug binds to the exact same binding site that glutamate reacts with.

As described in the Background section, candidate drugs which suppress receptor and neuronal activity, in this manner, usually are classified and labeled as anti-epileptic drugs, at least during the early stages of animal and clinical testing. Therefore, literature on these drugs can be found by searching the National Library of Medicine database for the terms AMPA, kainate, or glutamate, combined with the terms epileptic or anti-epileptic.

Accordingly, as mentioned in the Background section, drugs which selectively suppress KA receptor activity, and which can be evaluated for use as safener drugs as disclosed herein using no more than routine experimentation, include ACET, LY382884, ATPA, and 5-iodowillardiine (reviewed in articles such as Jane et al 2009), LU 97175 (e.g., Loscher et al 1999), and certain thieno-oxazine compounds (e.g., Briel et al 2009. Drugs which selectively suppress AMPA receptor activity, and which can be evaluated for use as safener drugs as disclosed herein using no more than routine experimentation, include NS1209 (e.g., Blackburn-Munro et al 2004), GYKI-52466 (e.g., Lees 2000), UBP277 and UBP282 (e.g., Ahmed 2009), LU 112313 (e.g., Loscher et al 1999), and talampanel (e.g., Rogawski 2006). Drugs which suppress activity at both KA and AMPA receptors without also suppressing activity at NMDA receptors, and which can be evaluated for use as safener drugs as disclosed herein using no more than routine experimentation, include topiramate (e.g., Rosenfeld 1997), and LU 115455 and LU 136541 (e.g., Loscher et al 1999).

Finally, drugs which reportedly suppress activity at all three classes of glutamate receptors (i.e., NMDA receptors, KA receptors, and AMPA receptors) include LU 73068 (e.g., Potschka et al 1998). It likely will be of interest, eventually, to test this drug and any others like it as potential alternatives to ketamine, in the types of prolonged and continuous NMDA antagonist treatments disclosed herein, since they may provide a class of NMDA antagonists which will have at least some degree of inherent safening activity. However, that approach is not preferred herein, since it poses significant and potentially serious risks of imbalances and unwanted effects which cannot be predicted, among populations having different ancestries and genotypes that may cause significant variations in receptor sequences, receptor affinities, receptor expression levels, etc., among the different types of glutamate receptors. For at least the time being, the ability to individually titer, adjust, and balance the dosages of both: (i) an NMDA-receptor-specific antagonist drug, such as ketamine, until it creates symptoms or sensations of mild inebriation; and, (ii) one or more specific safener drug(s) which suppress activity levels at KA and/or AMPA receptors but not NMDA receptors, until the psychotomimetic side effects of the NMDA antagonist drug are suitably controlled, is believed to provide a more flexible, adaptable, powerful, and more useful approach to providing the types of brain-altering treatments disclosed herein, than attempting to use a single drug in an attempt to have it perform multiple roles simultaneously.

Safener Category 1B: Agents that Suppress Activity at Non-NMDA Glutamate Receptors by Inhibiting Glutamate Release

As mentioned above, Category 1 (which includes subcategories 1A and 1B) includes drugs that suppress activity at either or both of the two non-NMDA classes of glutamate receptors (i.e., kainic acid (KA) receptors, and/or AMPA receptors).

One group of drugs which will suppress activity at both KA and AMPA receptors, simultaneously, acts by suppressing the release of glutamate from synaptic terminals. This mechanism is straightforward; if lower quantities of glutamate, the natural excitatory neurotransmitter, are released into synaptic junctions that contain non-NMDA glutamate (NNG) receptors, then those NNG receptors will not be activated as frequently.

Several known drugs accomplish this by inhibiting the action of sodium ion channels controlled by “upstream” synaptic receptors, in a manner which reduces the number of “firing” events that neurons undergo over a span of time. Drugs which act by this mechanism include riluzole, lamotrigine, carbamazepine, and diphenylhydantoin. Riluzole has been reported to help relieve the symptoms of depression in at least some cases; accordingly, it offers a good candidate for inclusion as a safener drug in NMDA antagonist treatments designed to treat severe depression. Lamotrigine reportedly has a significant level of activity in relieving neuropathic pain; accordingly, it offers a good candidate for inclusion as a safener drug in NMDA antagonist treatments designed to treat neuropathic pain.

Two other drugs which act in a similar manner, but which suppress activity at neuronal calcium channels rather than sodium channels, are gabapentin and pregabalin, described in articles such as Guay 2005, Chiechio et al 2009, and Dauri 2009. Gabapentin (sold under the trademark NEURONTIN) is an older drug, and pregabalin (sold under the trademark LYRICA) is regarded as a newer and more sophisticated drug. Pregabalin was initially approved by the FDA for use in controlling epilepsy, but it was discovered to also be effective in reducing and controlling certain types of neuropathic and post-operative pain that are resistant to other treatments. Accordingly, gabapentin and pregabalin are used for various types of pain control. Because they can suppress activity at NNG receptors and can also provide some measure of pain relief, they are good candidates for use as safener drugs in NMDA antagonist treatments designed to treat neuropathic pain.

The comments made below, regarding drugs that can suppress ACh release by agonist or potentiating activity at GABA receptors, also have potential relevance concerning suppression of glutamate release. However, because of the tendency of most GABAergic agents to cause drowsiness, those generally do not offer a preferred route for suppressing activity at non-NMDA receptors, at least during the early stages of testing to identify truly potent and effective safener drug combinations.

In addition, topiramate also reportedly can suppress neuronal activity, apparently via several different modes of action, including activity at sodium channels (e.g., Nakamura 2000). Because it has at least two other potentially important modes of action, involving (i) agonist and/or potentiating activity at GABA receptors, and (ii) antagonist activity at both KA and AMPA receptors, it is often referred to as a “multi-valent” drug. As noted elsewhere herein, drugs with multiple modes of action (this category also includes budipine, described in articles such as Przuntek et al 1999 and Reichmann 2006) generally are not preferred for early testing to identify potent and effective safener combinations, because the presence of multiple modes of actions, if introduced by a single drug, will introduce additional variables and complications into any resulting analyses. However, after the principles and concepts disclosed herein have been established, to a point of justifying government approval and widespread use of safener combinations, such “multi-valent” drugs will become of interest, and will merit serious attention in efforts to optimize subsequent drug regimens that are custom-tailored for treating specific types and categories of neurologic disorders.

Safener Category 2A: Agents that Suppress Activity at Muscarinic Ach Receptors by Competitive Binding

Safener drugs that fall within this Category 2A include agents which, as their primary mode of action, bind to and occupy the muscarinic class of acetylcholine receptors. Numerous drugs which bind to muscarinic ACh receptors are known, and include scopolamine, procyclidine, ethopropazine, piperidin, trihexyphenidyl, and numerous other known anti-cholinergic drugs.

Scopolamine is one of the more potent anti-cholinergic drugs. It has been known for decades, it is readily available, and it has a long history of use for preventing motion sickness, and for preventing nausea as a side effect of surgical anesthesia. Accordingly, it offers a preferred “baseline” and “benchmark” compound, for evaluation and use as disclosed herein. Any other candidate anti-cholinergic agent which is being evaluated for use as a safener drug in conjunction with ketamine (or any other NMDA antagonist) can be conveniently ranked, for efficacy, by comparing it to the efficacy of scopolamine, when used as disclosed herein.

A number of anticholinergic agents have chemical structures known as “aryl-cyclo-alkanolamines” (ACAA's). As indicated by that name, these drugs have an aromatic (or aryl) ring (such as benzene or a variant thereof) and a non-aromatic ring (such as cyclo-hexane), coupled to a short-chain alkyl group which has a pendant hydroxy group (i.e., an alkyl-alcohol, or alkanol); this alkanol component is also bonded to an amine structure. Examples of anti-cholinergic drugs with these structures include procyclidine, trihexyphenidyl, biperiden, triperiden, glycopyrrolate, hexocylium, oxyphenonium, tridihexethyl, and oxyphencyclomine. These agents formerly were used to treat patients suffering from Parkinson's disease or Parkinson-like symptoms, but they fell into relative disuse as other, more modern drugs were developed. Other drugs with chemical structures that are similar to the ACAA structure, and which competitively bind to muscarinic receptors, include mepenzolate (sold under the trademark CANTIL), piperodolate (DACTIL), isopropamide (DARBID), thiphenamil (TROCINATE), adiphenine (TRASENTINE), dicyclomine (BENTYL), and poldine (NACTON).

Another class of drugs which bind to muscarinic receptors have structures referred to as tri-cyclo-alkyl-amines (TCAA's). In this class of drugs the “tri-cyclic” structure has three rings, bonded to each other in a generally planar manner. Frequently, either or both of the two outside rings are aromatic, and the two atoms which bond them to each other may be either carbon, or another atom (for example, a tricyclic compound called phenothiazine has a nitrogen atom, and a sulfur atom, which effectively form a central ring that is positioned between two benzene rings). To create a true TCAA compound, an alkyl group and at least one nitrogen atom must also be bonded to the tricyclic component. TCAA drugs which are known to act as anti-cholinergic agents are exemplified by ethopropazine, which is sold under the trademark PARSIDOL.

Other types of tricyclic drugs which contain phenothiazine as their tricyclic structure have been developed, including an entire class of compounds that have chlorine or other strongly negative groups at their #2 positions. This increases their potency as neuroactive agents, and these compounds were used as anti-psychotic drugs when they were popular, which was mainly during the 1960's through 1980's. Examples of phenothiazine compounds used as anti-psychotic drugs include chlorpromazine (sold under the trademark THORAZINE), triflupromazine (VESPRIN), mesoridazine (SERENTIL), thioridazine (MELLARIL), acetophenazine (TINDAL), fluphenazine (PROLIXIN), perphenazine (TRILAFON), trifluoperazine (SUPRAZINE), and dixyrazine (ESOCALM). Their structures are shown in various references, such as the Merck Index, various review articles, and older editions of Goodman and Gilman. It is generally believed that their anti-psychotic activity was mainly due to their activity in blocking dopamine receptors; however, that activity caused Parkinson-like symptoms as a side effect, which led to the use of anti-cholinergic drugs (including ethopropazine) to help reduce and control the Parkinson-like symptoms.

In addition, various types of TCAA compounds have been commercialized, which have six-member center rings other than phenothiazine. Several tricyclic anti-psychotics used thioxanthene structures, with a sulfur atom but no nitrogen atom in a six-member center ring; examples include chlorprothixene, clopenthixol, flupentixal, piflutixol, and thiothixene.

Other TCAA compounds contain a tricyclic structure known as xanthene, which uses an oxygen atom and a carbon atom to form the center ring which connects two benzene rings. Examples include methantheline (BANTHINE), propantheline (PRO-BANTHENE), and maprotiline (DEPRILEPT).

In addition to the foregoing agents, a group of drugs that have tertiary amine groups, coupled to a tricyclic ring structure, also merit attention. These drugs include amitriptyline (sold under various trademarks, including ELAVIL), imipramine (sold under various trademarks, including DEPRINOL, IMIDOL, TOFRANIL, and IMIPREX), trimipramine, doxepin, clomipramine, and lofepramine (all sold under various trademarks that are listed in any recent edition of The Merck Index).

Another important member of the tricyclic tertiary amine group is olanzapine; in this molecule, the nitrogen atom that forms the tertiary amine component is part of a heterocyclic ring. Sold under the trademark ZYPREXA, olanzapine has become of major importance as an “atypical anti-psychotic” drug, used for treating psychosis, bipolar disorder, and major depressive disorders. It reportedly acts as an antagonist at several different dopamine, serotonin, and norepinephrine receptors. Olanzapine also acts as an antagonist (i.e., with effects that oppose and suppress the effects of the natural neurotransmitters) at the muscarinic class of ACh receptors, which are excitatory rather than inhibitory receptors. Olanzapine should be recognized as a successor drug which improved upon two predecessor drugs, clozapine and fluperlapine, which had the same types of receptor activities, but which caused a toxic and potentially lethal side effect in a small but significant subset of patients, involving a disorder called “agranulocytosis”.

During the Applicants' research into the safening properties of various anticholinergic agents, it was considered worth evaluating whether a tricyclic tertiary amine (amitriptyline was chosen as the candidate compound for testing) might also have safening properties. In unpublished experiments, the Applicants found that when amitriptyline was coadministered with a powerful NMDA antagonist, MK-801 (the maleate salt of dizocilpine), it did indeed provide substantial safening activity, which protected against the neurotoxic side effects of the MK-801. The Applicants have also found that amitriptyline has neuropathic pain-relieving activity, in animal models, when administered by itself; and, when coadministered along with memantine, a weak NMDA antagonist, amitriptyline potentiated the neuropathic pain-relieving activity of the memantine.

These results indicate that tricyclic tertiary amines (such as amitriptyline) offer exceptionally good candidates for use in combination with ketamine (or any other NMDA antagonist drug), in sustained subanesthetic treatment regimens for reducing neuropathic pain.

Furthermore, a large number of agents in the tricyclic tertiary amine class are also known to have anti-depressant activity; indeed, their main approved use is as anti-depressant drugs. Accordingly, they offer good candidates for use in combination with ketamine (or other NMDA antagonist drugs), in sustained subanesthetic treatment regimens for combating major depressive disorders.

In addition, quite a few other classes of anticholinergic agents have been reported, in respected and refereed journals, to have various neuroactive and/or psychiatric effects. For example, numerous tricyclic drugs which do not have pendant tertiary amine groups have anti-depressant, anti-psychotic, anti-Parkinson's, or similar effects; scopolamine and piperidin have been reported to have at least some antidepressant activity; and, procyclidine offers relief for neuropathic pain, in animal models.

Accordingly, any such drug which has substantial activity as a competitive binding agent at the muscarinic class of ACh receptors, and which has a known neuroactive or psychiatric effect that would be helpful in treating patients suffering from a specific type of neurologic disorder involving NMDA receptor hyperactivity and/or hypersensitivity, can be evaluated, using no more than routine experimentation once the teachings herein are known, for use as a safener drug to accompany ketamine (or any other NMDA antagonist drug) in a prolonged treatment regimen such as disclosed herein.

Safener Category 2B: Agents that Suppress Activity at Muscarinic ACh Receptors by Inhibiting ACh Release

A first major group of candidate drugs of interest herein, which can suppress the frequency at which neurons release acetylcholine (ACh, the natural excitatory neurotransmitter), function as agonists at alpha (α) adrenergic receptors, and particularly at the so-called α2 class of adrenergic receptors. This group of drugs includes, for example, clonidine, iodoclonidine, guanabenz, xylazine, medetomidine, tizanidine, rilmenidine, alpha-methyldopa, alpha-methylnoradrenaline, guanfacine, dexmedetomidine, azepexole, and lofexidine. As mentioned in the Background section, studies on neuronal circuitry, and on the effects of various drugs in mammalian brains, have shown that these α2 adrenergic agonist drugs have the effect of reducing the quantity of ACh that is released into synaptic junctions, by neurons in the brain. Accordingly, by suppressing the synaptic release of ACh inside the brain, α2 adrenergic agonists (such as clonidine) can reduce and suppress excitatory activity at muscarinic ACh receptors. One agent in this class (clonidine) has been shown to have neuropathic pain relieving activity and, when administered together with MK-801, clonidine potentiates MK-801's neuropathic pain-relieving activity.

A second group of Category 2B candidate drugs that potentially are of interest herein suppress ACh release by means of agonist activity at GABA receptors, especially at GABA-A receptors. In general, this class of agents is not regarded as being of primary interest for the early testing of various candidate safener combinations, due to the tendency of GABAergic agents to either:

(i) cause drowsiness, sedation, and even a loss of consciousness, in the case of “direct” GABA agonists, which include various barbiturates (such as pentobarbitol and thiamylal), propofol (a surgical anesthetic), etc.; or,

(ii) have mood-altering effects, in the case of “indirect” GABA agonists, such as benzodiazepines.

However, it should be kept in mind that the mood-altering effects of various benzodiazepine drugs, including diazepam (sold under the trademark VALIUM), include anxiolytic and tranquilizing effects. Therefore, if those types of effects are desired during sustained NMDA antagonist treatment of patients with certain types of neurologic disorders, then GABAergic agents that fall within Category 2B may become of interest, and may become especially useful.

Accordingly, the four categories of drugs that are listed above and in Table 1 contain numerous known drugs that offer good candidates for combined usage in synergistic combinations of safener drugs that, when used together to suppress activity at both muscarinic ACh and non-NMDA receptors, will provide exceptionally potent safening activity, even when large and/or prolonged NMDA antagonist dosage regimens are used.

### Testing of Drug Combinations in Small Animals

The subsections below contain comments on the use of specific types of tests in animals (and, subsequently, in humans) to identify specific safener combinations which will provide optimal safening activity when NMDA antagonists are used in heavy and/or prolonged dosages, to treat various types of neurologic disorders involving NMDA receptor hyper-activity, as described in the Background section. Those tests can be carried out using no more than routine experimentation, without requiring any undue creativity, by anyone who has read and studied the disclosures and invention herein.

The following comments apply to these types of tests:

1. To ensure close correlations between in vitro (cell culture) tests, animal tests, and actual use on humans, any research involving candidate safener drugs as described herein should use ketamine, as the NMDA antagonist drug that will be tested, unless strong reasons indicate otherwise. At the current time, ketamine is the only NMDA antagonist drug with “moderate” potency (i.e., greater than memantine, but lower than phencyclidine or MK-801) that is already approved for use in humans, and it begins with a “knowledge base” that arises from multiple decades of usage in humans. Therefore, unless and until some alternate NMDA antagonist with comparable “moderate” potency goes through a long and complex process of clinical trials and review, ketamine almost certainly will be the drug that is used, as an NMDA antagonist, in the types of prolonged treatments described herein.

Accordingly, rather than raising questions about whether (and to what extent) research data gathered by testing phencyclidine, MK-801, or memantine are truly relevant and revealing, it is far preferable to simply use ketamine during all testing as described herein, to ensure consistent results among different tests, and among different research teams.

2. A second major factor which needs to be addressed arises from the fact that ketamine has a “chiral” carbon atom (i.e., a carbon atom with four different types of atoms or molecular groups bonded to it). This means that there are two different “stereoisomers” (also called enantiomers) of ketamine, with structures that are “mirror images” of each other. Those two stereoisomers are usually called the S and R isomers, or the S(+) and R(−) isomers. Beginning in the mid-1990's, researchers in Germany began synthesizing and testing each isomer separately, and they reported (e.g., Albrecht et al 1996; Adams et al 1997) that S-ketamine appears to have two major advantages, compared to R-ketamine or a “racemic” mixture (i.e., an uncontrolled mixture which presumably contains roughly equal quantities of both the S isomer and the R isomer):

(i) S-ketamine reportedly is about twice as potent (as an anesthetic) as R-ketamine; and,

(ii) S-ketamine also is “cleared” from the body more rapidly than R-ketamine, mainly by metabolism in the liver; this enables better control of ketamine anesthesia, when the S(+) isomer is used during surgery.

Accordingly, ketamine formulations containing an essentially pure version of the S(+) isomer have become available, under the trademark KETANEST. While it may not be essential to use the purified S(+) isomer for a short-term episodic use, such as for anesthesia during a typical surgical operation lasting only a few hours, more serious questions concerns will arise if ketamine is being continuously infused into someone for a prolonged period of time, such as for 5 days and nights, continuously. Therefore, presumptions arise that:

(i) S-ketamine will be preferable to racemic ketamine, for prolonged infusion regimens in humans; and,

(ii) to ensure maximally comparable results, it generally would be advisable and preferable to carry out all testing of any candidate safener drug combinations, in humans, using the S-ketamine stereoisomer (rather than racemic mixtures) as the NMDA antagonist drug.

A number of policy statements and rules, issued by the Food and Drug Administration beginning in May 1992, effectively require pharmaceutical companies to address, study, and disclose data concerning different stereoisomers of any drugs that have such isomers. Those policy statements and rules should be reviewed and taken into account by anyone planning to do any research as disclosed herein with either S-ketamine or racemic ketamine, and any clinical trial results that are published should clearly state whether S-ketamine or racemic ketamine was used in the tests.

However, those issues do not directly affect research in animals, and racemic ketamine is much less expensive than S-ketamine. Furthermore, if rats are used as the test animals, it should be noted that their liver enzymes tend to metabolize and detoxify a wide range of foreign substances more actively and aggressively than in humans, presumably because the evolutionary niche of rats required them to be able to survive on an exceptionally wide variety of food sources, including carcasses, wastes, and other materials that had reached advanced stages of decay.

Accordingly, if tests on rats or other small animals are being used to evaluate the safening potency of various combinations of different candidate safener drugs, it does not appear to be essential (or even strongly preferable) to use S-ketamine in such tests. However, confirmatory tests will be performed, in rats, by the Applicants herein, which directly evaluate the safening potency of several fixed and unchanging safener combinations, against both (i) S-ketamine, in some tests, and (ii) racemic ketamine, in other tests, to confirm that the choice of S-ketamine, or racemic ketamine, in rat tests, will not substantially affect or alter the safener-related findings from such tests.

### Evaluating Safener Combinations in Small Animals

There are several ways that combinations of candidate safener drugs can be tested in animals, to measure and quantify the potency of any such combination in preventing the neurotoxic side effects of ketamine. Two such approaches can be briefly summarized as follows:

(1) test lower and lower dosages of safener combinations, to determine the lowest dosage, for each of various candidate safener combinations, which can protect against a fixed “challenge” created by an unchanging and relatively heavy dose of ketamine; or,

(2) test fixed and unchanging dosages of two candidate safener drugs, against increasingly greater dosages of ketamine, to determine which safener combinations can provide the best protection against very heavy dosages of ketamine.

A set of protocols for carrying out each of these two approaches is summarized below. Those skilled in the art will recognize variations on these tests, as well as various similar approaches that can accomplish essentially the same results in different ways.

Initial tests can utilize small animals, such as rats. If desired, subsequent tests can use larger mammalian species, including small monkeys or other primates; however, in view of various additional disclosures herein, about correlations between (i) vacuole formation in animal brains, and (ii) hallucinatory and other psychotomimetic effects in humans, it may not be necessary to perform tests on non-human primates or other non-rodent species.

The types of tests described below are designed to utilize “ED100” numbers, which refers to an “effective dosage” that creates a certain result (which can also be called an effect, display, manifestation, “end point”, etc.) in 100% of all animals tested. By using an approach which relies upon ED100 dosages, two desirable results can be achieved:

(1) a smaller number of animals can be used in each “population group” (or sample size, test group, or similar terms) that is treated in a certain manner. For example, sample sizes that contain only 4 rats in each group can provide valid ED100 numbers; by contrast, most scientists and statisticians generally assert that population groups of at least 6 (and preferably at least 10 or more) must be tested, to calculate statistical values (such as “mean” and “standard deviation” numbers) that have a sufficient level of reliability and “confidence”.

(2) if an approach that uses ED100 numbers is used, it will minimize the risk of distortions and mistakes that can enter into an experiment, when subjective interpretations are required. For example, if “vacuole formation, yes or no” is used as an “end point” which is evaluated for each animal, it becomes relatively simple and straightforward to determine whether vacuoles either are, or are not, seen under a light microscope, in stained tissue slices. By contrast, if the size, number, density, or other traits of any vacuoles which are found must be measured and analyzed, then various questions and problems of interpretation can arise, which can render the results less consistent less reliable, and more subject to challenges and dispute, especially if the results are generated by different research groups working at separate locations.

The Applicants herein have found that consistent and reliable results can be obtained, by sampling 3 different tissue slices from each brain; each slice should have a 1 micron thickness, and should be harvested from a brain region (such as the posterior cingulate or retrosplenial (PC/RS) cortex of the brain) which is known to generate large numbers of vacuoles when potent or high-dosage NMDA antagonists are administered. Furthermore, each of the sampled slices should be separated from the closest adjacent slice by a distance of 100 microns.

Furthermore, to provide the best and most sensitive analyses, the ED100 dose of ketamine which should be used consistently, in all tests in any specific lab, should cause a substantial but not exceptionally high or excessive number of vacuolated neurons, in all treated animals.

### First Method for Evaluating Safener Combinations, in Rats

A first preferred approach for testing various combinations of candidate safener drugs, in rats, will use lower and lower dosages of various candidate safener combinations, to determine the lowest dosage combinations that can still provide 100% protection against vacuole formation caused by ketamine. These types of tests can be carried out by steps such as the following:

Step 1: Administer the NMDA antagonist (preferably ketamine, either in racemic form or as S-ketamine; the chosen formulation must be used consistently through the entire set of tests) to aging female Sprague Dawley rats (each treatment or control group should include at least 4 rats), by intraperitoneal (IP) injection, at a dose sufficient to induce a vacuole reaction in the retrosplenial cortex of 100% of the treated rats. Control rats will receive normal saline in these “baseline” tests, and will not display a vacuole reaction in any of the treated rats. In the Applicants' laboratories, it has been determined that a racemic ketamine dosage which will induce a vacuole reaction in 100% of treated rats is 40 mg/kg, when administered by IP injection.

Step 2: Administer ketamine at its ED100 dosage (such as 40 mg/kg, IP), together with a range of doses of a candidate “Safener Drug A”. The results will establish a dosage that prevents any detectable ketamine-induced vacuole reaction, in 100% of the treated rats. This dosage of “Safener Drug A” can be designated by a term such as ED100-A.

Step 3: Repeat step two, using several other candidate safener drugs, which can be designated as B, C, D, etc., to determine an ED100-B, ED100-C, ED100-D, or similar dosage for each drug, which will indicate the dosage, for each candidate safener drug, that will block vacuole formation in all treated rats that are given the ED100 dosage of ketamine.

Step 4: Selected combinations of safener drugs are tested, over a range of increasingly lower dosages, against a fixed and unchanging “ketamine challenge”, to identify combinations which can protect against the ketamine challenge, even at relatively low dosages. For example, a first round of tests can test each candidate safener combination at 70%, 50%, and 30% portions (calculated by weight) of the ED100 dosages for each of the two safener drugs. If a two-drug combination, tested at 30% of each of their ED100 dosages, provides complete protection against vacuole formation, even lower percentages can be tested. These tests will identify safener combinations which can provide 100% protection against vacuole formation even at very low combined dosages, and that is a good indicator of high levels of potency.

If desired, in these types of tests, the safener drug combinations can be tested against the ED100 dosage of ketamine. Alternately or additionally, to provide a more difficult and “stiffer” challenge, which may help identify safener combinations which act in a truly synergistic (rather than merely additive) manner, the dosage of ketamine that is used for a series of direct-comparison tests can be increased by a chosen factor, which should be kept constant for that series of tests. For example, the “challenge” dosage of ketamine which is used to test various safener combinations can be increased to 120% (calculated by weight, in milligrams of drug per kilogram of animal body weight), or 150%, or 180% of the ED100 dosage. For example, if the ketamine dosage is increased by an additional 50% (calculated by weight) of the ED100 level, this increased ketamine dosage can be referred to, for convenience, as an ED150 dosage.

With regard to using heavier dosages of ketamine to create “stiffer” challenges, one of the goals is to create data which will sit somewhere generally within the “midrange” of the data which pertain to an effect or result that is being measured and evaluated. When test data are being gathered which will approximate a “bell curve”, the extreme “tail” regions (at the far left and right ends of the bell curve), are subject to problems of interpretation, where just a few data points in those outlying regions can create apparent and in some cases serious and misleading distortions. Accordingly, this type of statistical analysis becomes more reliable, and generates higher levels of confidence, if the parameters of a test are designed and adjusted so that the data which are being gathered will be arrayed reasonably close to the center a bell curve (or the point of the steepest upward or downward slope in an S-shaped curve).

As described above, primary attention, in these types of tests, should focus on two-drug combinations where: (1) one of the drugs is chosen because it can suppress activity at the muscarinic class of acetylcholine receptors, and (2) the other drug is chosen because it can suppress activity at non-NMDA glutamate receptors.

### Second Method for Evaluating Safener Combinations, in Rats

As mentioned above, a second preferred approach for testing various combinations of candidate safener drugs, in rats, involves (i) holding the dosages of safener drug combinations constant, and (ii) testing those combinations against increasing dosages of ketamine. These types of tests can be carried out by steps such as the following:

Step 1: Determine the ED100 vacuole-forming dosage for ketamine (if not done previously).

Step 2: Determine the ED100 safening dosage for each of several candidate safener drugs (if not done previously), which can be designated as drugs A, B, C, etc.

Step 3: Select one or more fixed and unchanging dosages, for a set of safener drug combinations. For example, an “A50-B50” safener drug combination can be created by using 50% (calculated by weight) of the ED100 dosages of each of candidate safener drugs A and B. Similar A50-C50, B50-C50, and other combinations can also be created and tested. Alternately, if 60%, 70%, or any other percentage or fraction is selected for testing, it should be used consistently, among all safener combinations that will be tested.

Step 4: Each candidate safener combination is tested against a series of increasingly greater dosages of ketamine (such as, for example, ketamine dosages equal to 120%, 140%, and 160% of the ED100 dosage of ketamine).

Accordingly, the objective is to identify those particular safener drug combinations which can provide protection against even relatively high dosages of ketamine. As with the first approach, primary attention should focus on two-drug combinations where: (1) one of the drugs is chosen because it can suppress activity at the muscarinic class of acetylcholine receptors, and (2) the other drug is chosen because it can suppress activity at non-NMDA glutamate receptors.

It is expected and anticipated, by the Applicants herein, that these and similar tests will generate data which make it clear that combinations of certain safener drugs will provide exceptionally good protection against ketamine-induced vacuole formation, including certain combinations which will rise to a level of synergistic rather than merely additive benefits. It is also believed and anticipated, by the applicants herein, that the best and most potent levels of safening activity are likely to arise when two different safener drugs are combined, which function by simultaneously limiting and controlling excessive activity at both (i) non-NMDA receptors, and (ii) muscarinic ACh receptors. Those particular combinations, which can be identified through relatively rapid, simple, and inexpensive testing in rats, will merit additional testing in larger animals and/or humans.

As will be apparent to those skilled in this type of research, the testing approaches summarized above are certainly not the only types of animal tests that can be done, to generate data that will help identify combinations of various safener drugs which can act in a synergistic manner, to provide exceptionally effective protection against the types of neurotoxic stresses posed by heavy or prolonged administration of an NMDA antagonist such as ketamine. Nevertheless, it is believed that the tests outlined above, even if conducted only in rats, will be fully sufficient to enable researchers to identify particular safener drug combinations that will merit additional testing, in larger animals (including rabbits, dogs, or primates, preferably using animals that are involved in other areas of medical research which do not involve neurologic testing), and/or in human clinical trials.

### Relationships Between Neurotoxic and Psychotomimetic Side Effects

Based on their own original research, and on their analysis of a large number of published articles (including many that have not previously been correlated with each other, or with efforts to develop improved ways to use NMDA antagonist drugs safely), the Applicants herein assert that the following correlations have been shown, repeatedly and consistently, by scientific evidence which is now sufficient to support and justify the following conclusions:

(1) the same types of neuronal stresses and disruptions which cause NMDA antagonist drugs to create observable and quantifiable indicators of stress and dysfunction (such as vacuole formation, expression of heat shock proteins, etc.) in animal brains, will also cause NMDA antagonist drugs to trigger neuronal deaths and permanent brain damage, if either the dosage or the duration of administration of a potent or moderately potent NMDA antagonist drug is increased above certain threshold levels;

(2) in order to provide the types of lasting therapeutic benefits described herein, an NMDA antagonist drug (such as ketamine) which has sufficient potency to provoke lasting and even permanent alterations in a human brain, must be administered at a dosage-and-duration combination which will create unacceptably high risks of permanent brain damage, unless potent and effective steps are taken to bring those high levels of risk down to minimal and tolerable levels of risk, by using safener drugs;

(3) the potency and efficacy of a safener drug (or combination of safener drugs), in preventing observable and quantifiable indicators of stress and dysfunction in animal brains, is directly correlated with the ability of that same safener drug (or combination of safener drugs) in suppressing hallucinations and other observable psychotomimetic side effects, in humans.

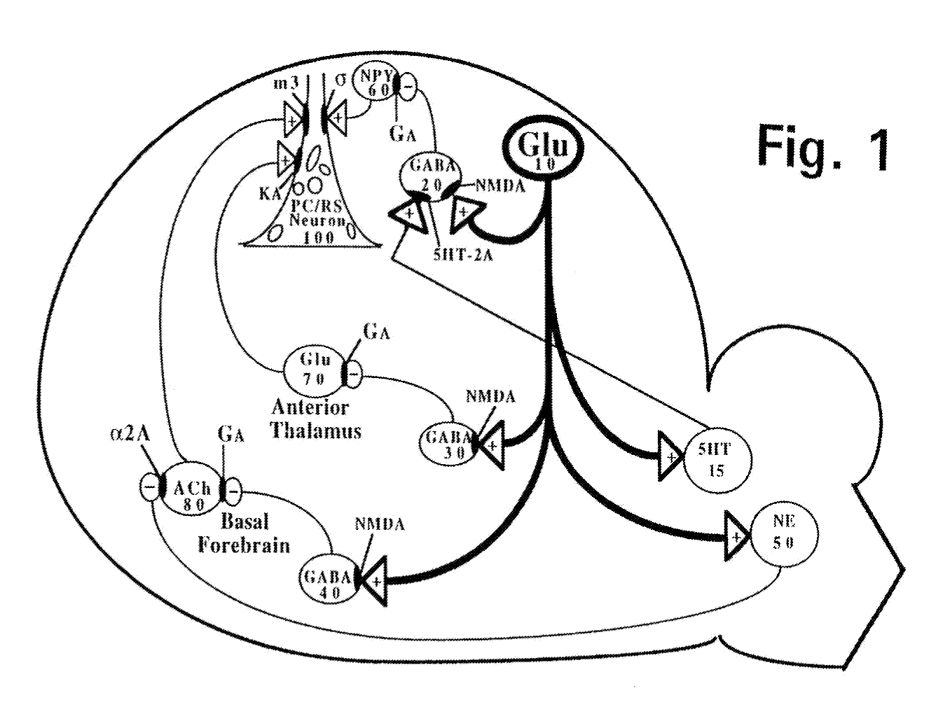

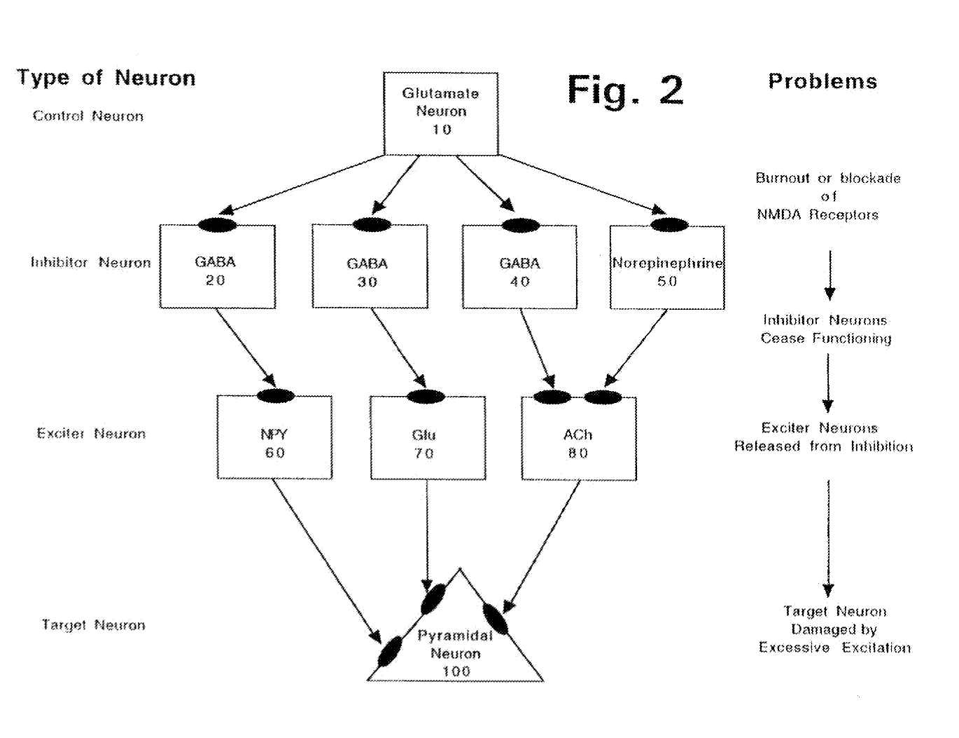

Although it is not apparent by superficial analysis, if one pieces together evidence from various sources and subjects that evidence to a careful analysis, it becomes evident that the same (or perhaps closely related and heavily overlapping) types of neuronal circuitry and network disturbances, disruptions, and derangements are responsible for both: (i) the neurotoxic effects seen in animals; and (ii) the psychotomimetic effects seen in humans, that are caused by NMDA antagonist drugs. Based on all evidence gathered to date, these types of circuitry and network disruptions are believed to arise from (or to at least include) the multi-factorial mechanisms that are illustrated, in schematic fashion, in FIGS. 1 and 2.

This interpretation is supported by two independent but parallel and in some respects overlapping sets of facts. These different sets of facts were gathered separately by a number of different research teams over the past years, and part of this current invention arises from the realization and understanding that, based on all known and available data, these separate sets of facts can and should now be directly correlated with and connected to each other.

The first cluster of facts arises from research showing that several specific classes of safener drugs, with known receptor activities, can indeed suppress and reduce, in measurable and quantifiable ways, the neurotoxic side effects of potent NMDA antagonists, in the brains of small animals such as rats.

The second cluster of facts arises from a growing body of research data (including not-yet-published data generated by the Applicants herein), which show that a number of those very same types of safener drugs will also suppress hallucinations and other psychotomimetic side effects, in humans. Despite the determined reluctance of most neurology researchers to publicly endorse or embrace any assertion or conclusion which states that the rat data gathered by the Applicants herein raises serious questions and doubts about the safety of a drug that is still widely used by anesthesiologists, a very strong correlation, which has recently emerged and solidified from both published and unpublished data, is that the drugs which will suppress (and in some dosages prevent) the measurable neurotoxic effects of NMDA antagonists, in rat brains, are the very same drugs that also will suppress hallucinations, in humans.

In particular, the accumulating scientific data (both published and unpublished) indicate that both (i) neurotoxic effects, in animals, and (ii) psychotomimetic effects, in humans, are suppressed or prevented by:
(1) GABA-A agonists, including both benzodiazepines and barbiturates (Magbagbeola et al., 1974; Reich and Silvay, 1989; Olney et al., 1991; Ishimaru et al., 1995, Jevtovi c-Todorovic et al., 1997);
(2) alpha-2 adrenergic agonists, including clonidine and guanabenz (Farber et al., 1995b; Levanen et al., 1995; Newcomer et al., 1998; Handa et al., 2000);
(3) lamotrigine, a drug that suppresses release of glutamate at synaptic junctions (Farber et al., 2002b; Anand et al., 2000);
(4) clozapine, a drug that has strong anticholinergic activity as well as activity at several other receptor types (Farber et al., 1995a; Malhotra et al., 1997).

In addition, these two phenomena (i.e., neurotoxic effects in animals, and psychotomimetic effects in humans) share a curious and noteworthy age-dependent profile. In particular, the onset of clear and direct susceptibility to either phenomenon does not occur until late adolescence. Prior to late adolescence, blockade of NMDA receptors does not trigger a neurotoxic reaction in animals (Farber et al., 1995c) nor does it induce a psychotomimetic reaction in humans (Reich and Silvay, 1989).

The Applicants' analysis and interpretation of the above correlations and findings is that prolonged continuous blockade of NMDA receptors in the brain, and consequent hyperstimulation of cerebrocortical neurons (due to excessively high signaling inputs from large numbers of contributing neurons that have been released or disconnected from their normal inhibitory control mechanisms) will cause a sequence of manifestations. The first manifestation will be mild psychotomimetic effects, which is a reflection of a functional disturbance in the hyperstimulated cerebrocortical neurons. These symptoms will be experienced prior to any physical changes in cerebrocortical neurons, and can be viewed as a warning signal that abnormal and aberrant hyperstimulation of cerebrocortical neurons is occurring. These types of initial symptoms serve as a warning that continued blockade of large numbers of NMDA receptors will cause unwanted and unhealthy morphologic changes in the affected neurons. Initially, those changes are reversible, and the neurons will be able to recover from them and return to an apparently normal state; however, it is not clear whether passage through that type of unwanted altered condition, for even a temporary period, will impose lasting unwanted changes on the affected neurons, such as loss of, damage to, or alterations of synaptic connections with other neurons, which can occur even if a neuron survives a period of severe stress.

Furthermore, it is quite clear, from animal studies and from certain well-known properties and behaviors of neurons, that if the stresses continue, unabated, the morphologic changes will become irreversible within several hours, and will then become lethal, and neurotoxic, in ways that will lead to irreversible brain damage. For example, evidence generated in rats (e.g., Jevtovic-Todorovic et al 2001), using NMDA antagonist agents that are rapidly cleared from the body (such as nitrous oxide), suggest that irreversible changes inside animal brains begin to manifest within about 8 hours after persistent administration of the NMDA antagonist drug begins.

There is evidence suggesting that if hyperexcitation at either ACh muscarinic receptor types, or non-NMDA receptor types, can be eliminated or at least substantially reduced, that may be sufficient, in some cases, to prevent or at least reduce the risk and severity of neurotoxic morphological changes. However, based on their studies to date, the Applicants herein believe that if hyperexcitation is eliminated or reduced at only one of those receptor systems, continuing hyperexcitation at the other receptor system is not normal, healthy, or desirable, and is likely to lead to psychotomimetic or other functional disturbances, and possibly to irreversible and permanent damage to the brain. Accordingly, the Applicants herein believe and assert that, if a patient with a severe neurologic disorder is being treated by continuous and prolonged administration of ketamine or a similar NMDA antagonist drug, then simultaneous use of at least two distinct safeners, one of which will potently and effectively eliminate the risk of hyperexcitation at muscarinic ACh receptors, while the other potently and effectively eliminates the risk of hyperexcitation at KA and/or AMPA receptors (and preferably both), would be strongly advisable and heavily preferred, in order to bring the risk levels down to truly minimal and acceptable levels in a manner which can meet the “best known practices” standards that have come to apply in modern medicine.

In addition, the Applicants intend to evaluate various types of brain imaging technology, notably including functional magnetic resonance imaging (fMRI), to determine whether, and to what extent, data and pictures from those types of imaging tests can establish additional correlations between tests on animals, and tests on humans, involving NMDA antagonists with and without safener drugs.

### Modes of Administration

The safener mixtures disclosed herein can be formulated and packaged in any of several well-known and conventional forms, provided that the dosage of each agent included in any such formulation must be clear and apparent (such as by an appropriate label on the package) to any physician who will decide on a preferred dosage regimen for each specific patient. For example, well-known orally-ingestible formulations include:

(1) tablets, in which a binder material is included with the active ingredients, to create a composition which, when compressed into the shape of a tablet that can be conveniently swallowed, will retain that shape, and which will dissolve after ingestion;

(2) capsules, in which a surrounding two-component capsule is used to enclose a heterogeneous material, which can be either a powdered or granular formulation, or a liquid or paste-type material;

(3) coated tablets, which can be used to protect a compressed tablet against stomach acids, and which can be designed to dissolve only after the coated tablet has passed through the stomach and has reached the small intestines.

Other formulations (such as a powder that must be added, in a controlled quantity, to an ingestible liquid, or an orally-ingestible liquid such as a syrup-type formulation) are also known, and can be created and used if desired; however, those types of orally-ingested formulations are not well suited for establishing the type of careful control, over a dosage regimen, that needs to be established for a mixture of neuroactive safener drugs such as disclosed herein. Instead, “unit dosage” formulations such as tablets or capsules are preferred for use as described herein.

Since a sustained treatment regimen as disclosed herein, using a potent NMDA antagonist drug such as ketamine, should be carried out only in a hospital or clinic under conditions which allow continuous monitoring of a patient's vital signs, it is generally advisable to formulate a safener drug mixture, as disclosed herein, in orally-ingestible unit dosage formulations (such as tablets or capsules) that contain relatively low dosages, which typically will require at least two or more pills to be given to the patient each day. This “small dosage in each pill” approach can allow the care-giving staff (including physicians and nurses) to “fine tune” the dosage that is being given to any specific patient, by giving a suitable number of low-dosage tablets or capsules to a patient, during the course of each day, while adjusting the total daily dosage (i.e., the number of pills given to a patient during each day) in response to any effects that are reported by or observed in the patient.

Alternately, the safener mixtures disclosed herein can be formulated in injectable form, such as in sealed sterile bags that render a liquid suited for continuous intravenous infusion. The use of adjustable clamps, placed on flexible infusion tubes in a manner that allows the “drip rate” for any specific patient to be controlled across a wide range of potential dosages, is a standard and well-known mode of administration for continuous infusion, and can be used for injectable liquids as disclosed herein. There is no need to administer a safener drug mixture, as disclosed herein, via a separate tube that will be attached to its own distinct hypodermic needle; instead, an infusible safener drug mixture as disclosed herein can be added to the infusion bag which contains the NMDA antagonist drug (such as ketamine) that is being administered to the patient. If tests indicate that the mixing of the two distinct liquids, inside an infusion bag, must be promoted by active mixing, then any of various known mixing means can be used. For example, to avoid any direct (and potentially contaminating) contact between a mixing device and an infusion liquid, a pair of small external rollers can be provided which will travel in a repeating or reciprocate manner, along the length of the infusion bag, in a manner comparable to a peristaltic pump which drives fluid through a flexible tube by means of moving pinch rollers that squeeze the outside of the tube.

Any drug that is listed herein as a candidate drug can be formulated as a “pharmacologically acceptable” salt or prodrug. Many of the neuroactive compounds disclosed herein tend to be mildly acidic or alkaline, and will dissociate when dissolved in an aqueous solution, in a manner which releases both (i) a neuroactive cation or anion, and (ii) a relatively unimportant single-atom anion or cation, which can be either a mineral (such as Na⁺, Cl⁻) or an organic ion (such as an acetate or maleate ion) which can be readily metabolized and used by the body. Accordingly, the use of salts to create “stabilized” formulations (often with increased and improved shelf-lives) of pharmaceutically active agents is a well-known practice, and can be used with the active agents discussed herein.

The term “prodrug” refers to a compound which is ingested or ingested in one form (which usually is inactive or only mildly active), which is then metabolized (usually by cleavage of a certain moiety from the initial compound, which will be catalyzed by one or more enzymes) inside the body, to create or release a fully active molecule. Frequently, prodrugs are used to create sustained-dosage formulations, since the metabolic “activation” of the active molecules which have entered the body will not occur immediately, and all at once. This approach has become well-known in the pharmaceutical arts, and can be used, if desired, to deliver any specific active agent of interest herein, if conditions indicate that administration of a prodrug, rather than a drug in an already-active form, is useful and advisable.

Other approaches also can be used to create sustained-dosage formulations, especially when orally-ingested formulations are involved. For example, an assortment of different binder materials and “microencapsulation” materials are known, which will dissolve at different rates, after ingestion. Accordingly, an encapsulated granular formulation can be created which will contain, for example, equal portions of three different types of granules, where the selection of three different binder or encapsulation materials will cause and enable ⅓ of the granules to dissolve rapidly, after being released by the outer capsule, while another ⅓ of the granules will dissolve more slowly, such as within 2 to 4 hours after being released from the outer capsule, and the final ⅓ of the granules will dissolve even more slowly, such as mainly within a span of about 4 to 8 hours after being released from the outer capsule.

With regard to liquid formulations that can be administered continuously via intravenous infusion, there is no particular need for the use of sustained-dosage formulations. Instead, the use of controlled infusion rates, throughout an infusion regimen which might last for days, can provide the desired results.

Finally, the treatments disclosed herein can be administered simultaneously or sequentially with various other treatments that: (i) have been used in the past to help a patient cope with a disease or disorder, or (ii) may have beneficial neuroprotective or other medical effects. These issues arise and will require attention because any patient who is seriously considering this type of treatment for a chronic condition will very likely be taking various medications and/or nutritional supplements in an effort to alleviate the problem that requires an intervention of the type described herein. Any such other treatments should be carefully evaluated by the treating physician, who will need to assess, for any individual patient, the likelihood of unwanted complications or potential benefits that might arise from either: (i) suspending, reducing, or otherwise altering the dosage of one or more other drugs or supplements to which the patient has become accustomed, habituated, or reliant upon; or, (ii) commencing any other pharmaceutical or other treatment which the physician believes may be helpful.

Thus, there has been shown and described a new and useful means for combining different classes of safener drugs, in ways which will allow such mixtures and combinations to prevent, reduce, and control the unwanted and potentially neurotoxic side effects of potent NMDA antagonist drugs, more potently and effectively than was previously possible. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### REFERENCES

Adams H A, et al, “From the racemate to the eutomer: (S)-ketamine. Renaissance of a substance?” (in German), Anaesthesist December 46(12): 1026-42, 1997
Ahmed A H, et al, “Mechanisms of antagonism of the GluR2 AMPA receptor: structure and dynamics of the complex of two willardiine antagonists with the glutamate binding domain,” Biochemistry. 2009 May 12; 48(18):3894-903
Albrecht S, et al, “New intravenous anesthetics. Remifentanil, S(+)-ketamine, eltanolone and target controlled infusion,” (in German), Anaesthesist, December 45(12): 1129-41, 1996
Anand A, et al, “Attenuation of the neuropsychiatric effects of ketamine with lamotrigine: support for hyperglutamatergic effects of NMDA receptor antagonists,” Arch Gen Psychiatry. 2000 March; 57(3):270-6
Anand K J, Soriano SG, “Anesthetic agents and the immature brain: are these toxic or therapeutic?” Anesthesiology. 2004 August; 101(2):527-30
Aracava Y, et al, “Memantine blocks alpha7* nicotinic acetylcholine receptors more potently than NMDA receptors in rat hippocampal neurons,” J Pharmacol Exp Ther. March 312(3): 1195-205, 2005
Backonja M, et al, “Response of chronic neuropathic pain syndromes to ketamine: a preliminary study,” Pain. 1994 January; 56(1):51-7. Erratum in: Pain 1994 September; 58(3):433
Bartels M, et al, “Anesthesia and cognitive performance in children: no evidence for a causal relationship,” Twin Res Hum Genet. 2009 June; 12(3):246-53; discussion in Twin Res Hum Genet. 2009 December; 12(6):611-4
Bell, R F, et al, “Ketamine as adjuvant to opioids for cancer pain. A qualitative systematic review,” J Pain Symptom Manage. 2003 September; 26(3):867-75
Berman, R M, “Antidepressant effects of ketamine in depressed patients,” Biol Psychiatry. 2000 Feb. 15; 47(4):351-4
Bi, H, et al, “NMDA receptor subunit NR2A and NR2B messenger RNA levels are altered in the hippocampus and entorhinal cortex in Alzheimer's disease,” J Neurol Sci. 2002 Aug. 15; 200(1-2):11-8.
Blackburn-Munro G, et al, “Behavioural effects of the novel AMPA/GluR5 selective receptor antagonist NS1209 after systemic administration in animal models of experimental pain,” Neuropharmacology. 2004 September; 47(3):351-62
Briel D, et al, “Synthesis of thieno[2,3-d]oxazines and thieno[2,3-d]thiazines as subtype specific kainate receptor antagonists,” Curr Med Chem. 2009; 16(35):4704-11
Buisson B, et al, “Open-channel blockers at the human alpha4beta2 neuronal nicotinic acetylcholine receptor,” Mol Pharmacol, March 53(3): 555-63, 1998
Casarosa P, et al, “Preclinical evaluation of long-acting muscarinic antagonists: comparison of tiotropium and investigational drugs,” J Pharmacol Exp Ther. 2009 August; 330(2):660-8
Chen H S, et al, “The chemical biology of clinically tolerated NMDA receptor antagonists,” J Neurochem, June 97(6): 1611-26, 2006
Chiechio S, et al, “Pregabalin in the treatment of chronic pain: an overview,” Clin Drug Investig, 29(3): 203-13, 2009
Choi D W, “Excitotoxic Cell Death,” J Neurobiol 23, 1261-1276, 1992
Correll, G E, et al, “Subanesthetic ketamine infusion therapy: a retrospective analysis of a novel therapeutic approach to complex regional pain syndrome,” Pain Med. 2004 September; 5(3):263-75
Correll, G E, et al, “Two case studies of patients with major depressive disorder given low-dose (subanesthetic) ketamine infusions,” Pain Med. 2006 January-February; 7(1):92-5
Creeley C, et al, “Low doses of memantine disrupt memory in adult rats,” J Neurosci. 2006 Apr. 12; 26(15):3923-32
Creeley C E, et al, “Donepezil markedly potentiates memantine neurotoxicity in the adult rat brain,” Neurobiol Aging. 2008 February; 29(2):153-67
Crick F, et al, “REM sleep and neural nets,” Behav Brain Res. 1995 July-August; 69(1-2):147-55
Crick F, et al, “The function of dream sleep.” Nature 304 (1983): 111-114
Dauri M, et al, “Gabapentin and pregabalin for the acute post-operative pain management. A systematic-narrative review of the recent clinical evidences,” Curr Drug Targets, August 10(8): 716-33, 2009
DiMaggio C, et al, “A retrospective cohort study of the association of anesthesia and hernia repair surgery with behavioral and developmental disorders in young children,” J Neurosurg Anesthesiol. 2009 October; 21(4):286-91; comment in J Neurosurg Anesthesiol. 2009 October; 21(4):283-5
Dreyer B D, et al, “Memantine acts as a cholinergic stimulant in the mouse hippocampus,” J Alzheimers Disease, December 12(4): 319-33, 2007
Farber N B, et al, “Age-specific neurotoxicity in the rat associated with NMDA receptor blockade: potential relevance to schizophrenia?” Biol Psychiatry. 1995 Dec. 15; 38(12):788-96
Farber N B, et al, “Alpha 2 adrenergic agonists prevent MK-801 neurotoxicity,” Neuropsychopharmacology. 1995 July; 12(4):347-9
Farber N B, et al, “Antiepileptic drugs and agents that inhibit voltage-gated sodium channels prevent NMDA antagonist neurotoxicity,” Mol Psychiatry. 2002; 7(7):726-33
Farber N B, Olney J W, “Drugs of abuse that cause developing neurons to commit suicide,” Brain Res Dev Brain Res. 2003 Dec. 30; 147(1-2):37-45
Farber, N. B., et al, “Antipsychotic drugs block phencyclidine receptor-mediated neurotoxicity,” Biol Psychiatry 34: 119-121 (1993)
Fitzgibbon E J, et al, “Low dose ketamine as an analgesic adjuvant in difficult pain syndromes: a strategy for conversion from parenteral to oral ketamine,” J Pain Symptom Manage. 2002 February; 23(2):165-70
Gonzales R A, et al, “NMDA mediated responses decrease with age in Fischer 344 rat brain,” Neurobiol Aging. 1991 May-June; 12(3):219-25
Guay D R, “Pregabalin in neuropathic pain: a more ‘pharmaceutically elegant’ gabapentin?” Am J Geriatr Pharmacother, December 3(4): 274-87, 2005
Harbut, R E, et al, “Successful treatment of a nine-year case of complex regional pain syndrome type-I (reflex sympathetic dystrophy) with intravenous ketamine-infusion therapy in a warfarin-anticoagulated adult female patient,” Pain Med. 2002 June; 3(2):147-55
Hardingham G E, “Coupling of the NMDA receptor to neuroprotective and neurodestructive events” Biochem Soc Trans, 37: 1147-60, 2009
Hopfield J J, et al, “Unlearning has a stabilizing effect in collective memories,” Nature. 1983 Jul. 14-20; 304(5922):158-9
Horn, D, et al, “Neuronal regulation versus synaptic unlearning in memory maintenance mechanisms,” Network. 1998 November; 9(4):577-86
Hui et al, “Novel therapies for schizophrenia: understanding the glutamatergic synapse and potential targets for altering NMDA neurotransmission,” Recent Pat CNS Drug Discov, November 4(3): 220-38, 2009
Ishimaru M, et al, “Halothane prevents MK-801 neurotoxicity in the rat cingulate cortex,” Neurosci Lett 193: 1-4 (1995)
Iwamoto F M, et al, “Phase 2 trial of talampanel, a glutamate receptor inhibitor, for adults with recurrent malignant gliomas,” Cancer. April 1; 116(7): 1776-82, 2010
Jane D E, et al, “Kainate receptors: pharmacology, function and therapeutic potential,” Neuropharmacology. 2009 January; 56(1):90-113
Javitt 2004 “Glutamate as a therapeutic target in psychiatric disorders,” Mol Psychiatry, November 9(11): 984-97, 979, 2004
Jevtovic-Todorovic V, et al, “Prolonged exposure to inhalational anesthetic nitrous oxide kills neurons in adult rat brain,” Neuroscience. 2003; 122(3):609-16
Jevtovic-Todorovic V., et al, “Isoflurane and propofol block neurotoxicity caused by HK-801 in the rat posterior cingulate/retrosplenial cortex,” J Cereb Blood Flow Metab 1997
Johannessen-Landmark C, et al, “Drug interactions involving the new second- and third-generation antiepileptic drugs,” Expert Rev Neurother. 2010 January; 10(1):119-40
Joos G F, “Potential for long-acting muscarinic antagonists in chronic obstructive pulmonary disease,” Expert Opin Investig Drugs. 2010 February; 19(2):257-64
Kalkman C J, et al, “Behavior and development in children and age at the time of first anesthetic exposure,” Anesthesiology. 2009 April; 110(4):805-12, discussed in Anesthesiology. 2009 December; 111(6):1383-7 and Anesthesiology. 2009 April; 110(4):703-8.
Kannan T R, et al, “Oral ketamine as an adjuvant to oral morphine for neuropathic pain in cancer patients,” J Pain Symptom Manage. 2002 January; 23(1):60-5
Klepstad P, et al, “Long-term treatment with ketamine in a 12-year-old girl with severe neuropathic pain caused by a cervical spinal tumor,” J Pediatr Hematol Oncol. 2001 December; 23(9):616-9
Lahti A C, et al, “Ketamine activates psychosis and alters limbic blood flow in schizophrenia,” Neuroreport. 1995 Apr. 19; 6(6):869-72
Lahti A C, et al, “Subanesthetic doses of ketamine stimulate psychosis in schizophrenia,” Neuropsychopharmacology. 1995 August; 13(1):9-19
Lees G J, “Pharmacology of AMPA/kainate receptor ligands and their therapeutic potential in neurological and psychiatric disorders,” Drugs. 2000 January; 59(1):33-78
Levanen J, et al, “Dexmedetomidine premedication attenuates ketamine-induced cardiostimulatory effects and postanesthetic delirium,” Anesthesiology. 1995 May; 82(5):1117-25
Lipton S A, et al, “Excitatory amino acids as a final common pathway for neurologic disorders,” N. Eng. J. Med. 330 (9), 613-622, 1994
Lipton S A, et al, “Excitatory amino acids as a final common pathway for neurologic disorders,” N. Eng. J. Med. 330 (9), 613-622, 1994
Lipton S A, “Paradigm shift in NMDA receptor antagonist drug development: molecular mechanism of uncompetitive inhibition by memantine in the treatment of Alzheimer's disease and other neurologic disorders,” J Alzheimers Dis. 2004 December; 6(6 Suppl):S61-74.
Lipton S A, “The molecular basis of memantine action in Alzheimer's disease and other neurologic disorders: low-affinity, uncompetitive antagonism,” Curr Alzheimer Res. April 2(2): 155-65, 2005
Loscher W, et al, “A new pyrrolyl-quinoxalinedione series of non-NMDA glutamate receptor antagonists,” Eur J Neurosci. 1999 January; 11(1):250-62
Luszczki J J, “Third-generation antiepileptic drugs: mechanisms of action, pharmacokinetics and interactions,” Pharmacol Rep. 2009 March-April; 61(2):197-216
Magbagbeola J A, et al, “Effect of thiopentone on emergence reactions to ketamine anaesthesia,” Can Anaesth Soc J. 1974 May; 21(3):321-4
Magnusson K R, et al, “Effects of aging on NMDA and MK801 binding sites in mice,” Brain Res. 1993 Feb. 26; 604(1-2):334-7
Maskell P D, Set al, “Inhibition of human alpha 7 nicotinic acetylcholine receptors by open channel blockers of NMDA receptors,” Br J Pharmacol, December 140(7): 1313-9, 2003
McClaine R J, et al, “General anesthesia improves fetal cerebral oxygenation without evidence of subsequent neuronal injury,” J Cereb Blood Flow Metab. 2005 August; 25(8):1060-9
Mitchell A C, “An unusual case of chronic neuropathic pain responds to an optimum frequency of intravenous ketamine infusions,” J Pain Symptom Manage. 2001 May; 21(5):443-6
More J C, et al, “Structural requirements for novel willardiine derivatives acting as AMPA and kainate receptor antagonists,” Br J Pharmacol. 2003 March; 138(6):1093-100
Nakamura J, et al, “Target pharmacology of topiramate, a new antiepileptic drug,” (in Japanese) Nippon Yakurigaku Zasshi, January 115(1): 53-7, 2000
Okon T, “Ketamine: an introduction for the pain and palliative medicine physician,” Pain Physician. 2007 May; 10(3):493-500
Oliver D, et al, “Memantine inhibits efferent cholinergic transmission in the cochlea by blocking nicotinic acetylcholine receptors of outer hair cells,” Mol Pharmacol, July; 60(1): 183-9, 2001
Olney J W, “Excitatory amino acids and neuropsychiatric disorders,” Biol Psychiatry 26: 505-525, 1989
Olney J W, “Excitotoxicity and NMDA receptors,” Drug Dev Res 17, 299-319, 1989
Olney J W, “The neurotoxicity of NMDA receptor antagonists: An overview,” Psychopharmacol Bull 30, 533-540, 1994
Olney J W, Farber N B, “Glutamate receptor dysfunction and schizophrenia,” Arch Gen Psychiatry. 1995 December; 52(12):998-1007
Olney J W, Farber N B, “NMDA antagonists as neurotherapeutic drugs, psychotogens, neurotoxins, and research tools for studying schizophrenia,” Neuropsychopharmacology. 1995 December; 13(4):335-45
Olney J W, et al, “NMDA antagonist neurotoxicity: Mechanism and protection,” Science 254: 1515-1518 (1991)
Olney J W, “Fetal alcohol syndrome at the cellular level,” Addict Biol. 2004 June; 9(2):137-49; discussion 151
Patsalos P N, “Properties of antiepileptic drugs in the treatment of idiopathic generalized epilepsies,” Epilepsia. 2005; 46 Suppl 9:140-8
Paul I A, et al, “Glutamate and depression: clinical and preclinical studies,” Ann N Y Acad. Sci. 2003 November; 1003:250-72
Perucca E, “The new generation of antiepileptic drugs: advantages and disadvantages,” Br J Clin Pharmacol. 1996 November; 42(5):531-43
Piggott M A, et al, “[3H]MK-801 binding to the NMDA receptor complex, and its modulation in human frontal cortex during development and aging,” Brain Res. 1992 Aug. 21; 588(2):277-86
Pitkanen A, et al, “Effect of novel AMPA antagonist, NS1209, on status epilepticus: An experimental study in rat,” Epilepsy Res. April 74(1): 45-54, 2007
Pittenger C, et al, “The NMDA receptor as a therapeutic target in major depressive disorder,” CNS Neurol Disord Drug Targets. 2007 April; 6(2):101-15
Preskorn, S H, “An innovative design to establish proof of concept of the antidepressant effects of the NR2B subunit selective NMDA antagonist, CP-101,606, in patients with treatment-refractory major depressive disorder,” J Clin Psychopharmacol. 2008 December; 28(6):631-7
Przuntek H, et al, “Clinical efficacy of budipine in Parkinson's disease,” J Neural Transm Suppl. 56: 75-82, 1996
Rabben T, et al, “Interindividual differences in the analgesic response to ketamine in chronic orofacial pain,” Eur J Pain. 2001; 5(3):233-40
Reich D L, et al, “Ketamine: an update on the first twenty years of clinical experience,” Can J Anaesth 36: 186-197 (1989)
Reichmann H, “Budipine in Parkinson's tremor,” J. Neurol. Sci. 248 (1-2): 53-5, 2006
Rogawski M A, “Diverse mechanisms of antiepileptic drugs in the development pipeline,” Epilepsy Res. 2006 June; 69(3):273-94
Rosenfeld W E, “Topiramate: a review of preclinical, pharmacokinetic, and clinical data,” Clin Ther. 1997 November-December; 19(6):1294-308
Rothman S M and Olney J W, “Excitotoxicity and the NMDA receptor,” Trends In NeuroScience 10, 299-302, 1987
Rothman S M and Olney J W, “Glutamate and the pathophysiology of hypoxic-ischemic brain damage,” Ann Neurol 19, 105-111, 1986
Rzeski W, et al, “Glutamate antagonists limit tumor growth,” Biochem Pharmacol. 2002 Oct. 15; 64(8):1195-200
Sanacora G, “Do glutamatergic agents represent a new class of antidepressant drugs?” J Clin Psychiatry, 70: 1473-75, 2009.
Sanacora G, et al, “Subtype-specific alterations of gamma-aminobutyric acid and glutamate in patients with major depression,” Arch Gen Psychiatry. 2004 July; 61(7):705-13
Siegel J M, “The REM sleep-memory consolidation hypothesis,” Science. 2001 Nov. 2; 294(5544):1058-63
Skolnick P, et al, “Glutamate-based antidepressants: 20 years on,” Trends Pharmacol Sci. 2009 November; 30(11):563-9
Smith C T, et al, “Posttraining increases in REM sleep intensity implicate REM sleep in memory processing and provide a biological marker of learning potential,” Learn Mem. 2004 November-December; 11(6):714-9
Snyder E M, et al, “Regulation of NMDA receptor trafficking by amyloid-beta,” Nat Neurosci. 2005 August; 8(8):1051-8.
Soriano S G, Anand K J, “Anesthetics and brain toxicity,” Curr Opin Anaesthesiol. 2005 June; 18(3):293-7
Stepulak A, et al, “NMDA antagonist inhibits the extracellular signal-regulated kinase pathway and suppresses cancer growth,” Proc Natl Acad Sci USA. 2005 Oct. 25; 102(43):15605-10
Sze C, et al, “NMDA receptor subunit proteins and their phosphorylation status are altered selectively in Alzheimer's disease,” J Neurol Sci. 2001 Jan. 1; 182(2):151-9.
Tamaru M, et al, “Age-related decreases of the NMDA receptor complex in the rat cerebral cortex and hippocampus,” Brain Res. 1991 Feb. 22; 542(1):83-90
Ulas J et al, “Decreased expression of NMDA receptor 1 messenger RNA in select regions of Alzheimer brain,” Neuroscience. 1997 August; 79(4):973-82
Wada A, et al, “NR3A modulates the outer vestibule of the NMDA receptor channel,” J Neurosci. 2006 Dec. 20; 26(51):13156-66.
Wenk G L, et al, “Loss of NMDA, but not GABA-A, binding in the brains of aged rats and monkeys,” Neurobiol Aging. 1991 March-April; 12(2):93-8
Wilder R T, et al, “Early exposure to anesthesia and learning disabilities in a population-based birth cohort,” Anesthesiology. 2009 April; 110(4):796-804
Witkin J M, et al, “Metabotropic glutamate receptors in the control of mood disorders,” CNS Neurol Disord Drug Targets. 2007 April; 6(2):87-100
Xia P, et al, “Memantine Preferentially Blocks Extrasynaptic over Synaptic NMDA Receptor Currents in Hippocampal Autapses, J Neurosci. 2010 Aug. 18; 30(33):11246-50.
Zarate C A, et al, “A double-blind, placebo-controlled study of memantine in the treatment of major depression,” Am J Psychiatry. 2006 January;163(1):153-5
Zarate C A, et al, “A randomized trial of an NMDA antagonist in treatment-resistant major depression,” Arch Gen Psychiatry. 2006 August; 63(8):856-64
Zhao X, et al, “The effects of aging on N-methyl-D-aspartate receptor subunits in the synaptic membrane and relationships to long-term spatial memory,” Neuroscience. 2009 Sep. 15; 162(4):933-45.

1. A composition of matter, comprising a mixture of safener drugs that have been selected for their efficacy and potency in preventing unwanted side effects that are caused when potent NMDA antagonist drugs are administered without an accompanying safener drug, wherein said mixture of safener drugs comprises:
(i) at least one first safener drug which suppresses transmitter activity at non-NMDA glutamate receptors; and,
(ii) at least one second drug which suppresses transmitter activity at muscarinic acetylcholine receptors,
and wherein each of said first and second safener drugs has been shown, in animal tests and in human clinical tests, to reduce unwanted side effects of potent NMDA antagonist drugs,
and wherein said mixture of said first and second safener drugs has been shown to be more effective in reducing both:
(a) neurotoxic damage in animals, and
(b) psychotomimetic effects in humans,
than can be achieved by either said first or said second safener drug when administered as a single safening agent along with a potent NMDA antagonist drug.

2. The composition of matter of claim 1 wherein said first safener drug suppresses transmitter activity at non-NMDA glutamate receptors by competitive binding to non-NMDA receptors.

3. The composition of matter of claim 2 wherein said first safener drug is selected from the group consisting of 5-iodowillardiine, LY-382884, LU-97175, LU-112313, LU-115455, LU-136541, NS-1209, GYKI-52466, UBP-277, UBP-282, ACET, and ATPA.

4. The composition of matter of claim 1 wherein said first safener drug suppresses transmitter activity at non-NMDA glutamate receptors, by suppressing release of glutamate by central nervous system neurons.

5. The composition of matter of claim 4 wherein said first safener drug is selected from the group consisting of lamotrigine, riluzole, carbamazepine, diphenylhydantoin, topiramate, gabapentin, and pregabalin, and pharmaceutically acceptable salts thereof.

6. The composition of matter of claim 1 wherein said second safener drug suppresses transmitter activity at muscarinic acetylcholine receptors, by means of competitive binding to muscarinic acetylcholine receptors.

7. The composition of claim 1 wherein said second safener drug is selected from the group consisting of scopolamine, atropine, benztropine, benactyzine, biperiden, procyclidine, trihexyphenidyl, and diphenhydramine, and pharmaceutically acceptable salts thereof.

8. The composition of matter of claim 1 wherein said second safener drug is conventionally used as an anti-depressant, and has antagonist activity at muscarinic acetylcholine receptors.

9. The composition of claim 9 wherein the second drug is selected from the group consisting of amitriptyline, imipramine, trimipramine, doxepin, clomipramine, and lofepramine, and pharmaceutically acceptable salts thereof.

10. The composition of matter of claim 1 wherein said second safener drug suppresses transmitter activity at muscarinic acetylcholine receptors by suppressing release of acetylcholine by central nervous system neurons.

11. The composition of matter of claim 10 wherein said second safener drug acts as an agonist at alpha-2 adrenergic receptors.

12. The composition of matter of claim 11 wherein said second safener drug is selected from the group consisting of clonidine, iodoclonidine, guanabenz, xylazine, medetomidine, tizanidine, rilmenidine, alpha-methyldopa, alpha-methylnoradrenaline, guanfacine, dexmedetomidine, azepexole, and lofexidine, and pharmaceutically acceptable salts thereof.

13. The composition of matter of claim 1 wherein said first and second safener drugs are each present in an orally ingestible unit-dosage formulation selected from the group consisting of tablets and capsules.

14. The composition of matter of claim 1 wherein said first and second safener drugs are each present in an orally ingestible liquid mixture contained within a package which specifies (i) dosages for each of said first and second safener drugs, and (ii) recommended ingestion rate or frequency for said liquid mixture.

15. The composition of matter of claim 1 wherein said first and second safener drugs are each present in a liquid mixture which is maintained in a sealed sterile package and which is suited for continuous intravenous infusion.

16. The composition of matter of claim 1 wherein said mixture of safener drugs comprises:
(i) at least one first safener drug which suppresses transmitter activity at non-NMDA glutamate receptors; and,
(ii) at least one second safener drug which suppresses transmitter activity at muscarinic acetylcholine receptors by competitive binding to muscarinic acetylcholine receptors; and,
(iii) at least one third safener drug which suppresses transmitter activity at muscarinic acetylcholine receptors by suppressing release of acetylcholine by central nervous system neurons,
wherein each of said first, second, and third safener drugs has been shown, both in animal tests and in human clinical trials, to reduce unwanted side effects of potent NMDA antagonist drugs,
and wherein said mixture of said first, second, and third safener drugs has been shown, both in animal tests and in human clinical trials, to have synergistic potency in reducing unwanted side effects of potent NMDA antagonist drugs, at levels which cannot be achieved by clinically relevant dosages of any one of said first, second, or third safener drugs when administered as a sole safener drug.
